(19) United States
(12) Patent Application Publication
(10) Pub. No.: US 2016/0066857 A1
(43) Pub. Date: Mar. 10, 2016

(54) Device-Based Activity Classification Using Predictive Feature Analysis

(71) Applicants: Stuart Crawford, Piedmont, CA (US); Piyush Savalia, San Francisco, CA (US); Prasad Panchalan, San Francisco, CA (US); Sylvia Hou-Yan Cheng, San Francisco, CA (US); Chris Singleton, Palm Harbor, FL (US); Sheila Nabanja, San Jose, CA (US); Ilyas Mohammad, San Francisco, CA (US); Sumit Sharma, San Jose, CA (US)

(72) Inventors: Stuart Crawford, Piedmont, CA (US); Piyush Savalia, San Francisco, CA (US); Prasad Panchalan, San Francisco, CA (US); Sylvia Hou-Yan Cheng, San Francisco, CA (US); Chris Singleton, Palm Harbor, FL (US); Sheila Nabanja, San Jose, CA (US); Ilyas Mohammad, San Francisco, CA (US); Sumit Sharma, San Jose, CA (US)

(73) Assignee: AliphCom, San Francisco, CA (US)

(21) Appl. No.: 14/121,938

(22) Filed: Nov. 4, 2014

Related U.S. Application Data
(63) Continuation-in-part of application No. 14/480,628, filed on Sep. 8, 2014.

(51) Int. Cl. A61B 5/00; A61B 5/0205

(52) U.S. Cl. CPC A61B 5/7264; A61B 5/0205; A61B 5/7275; A61B 5/4812; A61B 5/742; A61B 5/7475; A61B 5/681; A61B 5/0245

(57) ABSTRACT

Device-based activity classification using predictive feature analysis is described, including receiving a signal from a sensor configured to measure a heart rate coupled to a device, the sensor being configured to sense the signal over a time period, evaluating the signal to generate data associated with the heart rate, the data being further evaluated to select a classifier, invoking the classifier, the classifier being configured to evaluate the data to identify a predictive feature, the predictive feature invoking an application configured to determine a state using a feature interpreter, the application also being configured to evaluate other data from another signal, the signal being configured to detect a respiration rate, and processing the data and the other data using the application and the feature interpreter to generate information associated with sleep, the information being configured to display on an interface associated with the device.

### FIELD

Embodiments relate generally to electrical and electronic hardware, computer software, wired and wireless network communications, and computing devices, and, in particular, to using predictive feature information and classifier functions in wearable devices.

### BACKGROUND

Wearable devices have leveraged increased sensor and computing capabilities that can be provided in reduced personal and/or portable form factors, and an increasing number of applications (i.e., computer and Internet software or programs) for different uses, consumers (i.e., users) have given rise to large amounts of personal data that can be analyzed on an individual basis or an aggregated basis (e.g., anonymized groupings of samples describing user activity, state, and condition).

Presently, development and design of many wearable devices, such as so-called “smart watches,” are including glass-based touchscreens to enable users to interact with glass (or transparent plastic) to provide user input or receive visual information. An example of a glass-based touch screen includes CORNING® GORILLA® GLASS, or those formed using OLED or other like technology. Developers of wearable devices using such touchscreens continue to face challenges, not only technically but also in user experience design. For example, relatively large glass-based touchscreens may be perceived to be to “bulky” or “unwieldy” for some consumers, whereas miniaturized glass-based screens may fail to provide sufficient information to a user. Moreover, some conventional touchscreens are susceptible to the environments in which users typically expect reliable operation.

Further, some conventional smart watches implement short range communication systems (e.g., transceivers and antennas) adjacent glass portions and/or plastic portions of a housing to interference from metal structures. While conventional wearable devices typically are functional, such devices have sub-optimal properties that consumers view less favorably.

Thus, what is needed is a solution for improving the efficacy and effectiveness of signal processing and data operations in wearable devices without the limitations of conventional devices or techniques.

### BRIEF DESCRIPTION OF THE DRAWINGS

Various embodiments or examples (“examples”) of the invention are disclosed in the following detailed description and the accompanying drawings:

FIG. 1 is a perspective view of a wearable device, according to some embodiments;

FIGS. 2A and 2B are diagrams depicting an exploded front view and an exploded perspective view, respectively, of a wearable device, according to some embodiments;

FIGS. 3 and 4 are flow diagrams depicting examples of flows for forming a cradle and forming an anchored cradle, respectively, according to some embodiments;

FIGS. 5A and 5B are diagrams depicting a front view and a perspective view, respectively, of an anchored cradle, according to some embodiments;

FIGS. 6A to 6C are diagrams depicting formation of an intermediate assembly structure formed in molding process, according to some examples;

FIGS. 7A to 7B are diagrams depicting formation of another intermediate assembly structure formed in molding process, according to some examples;

FIGS. 8A to 8C are diagrams depicting exploded views of logic, circuitry, and components disposed within the interior of a cradle anchored to two straps, according to some embodiments;

FIGS. 9A and 9B are diagrams depicting an assembly step in which one or more pod covers of a wearable pod are integrated into a wearable device, according to some embodiments;

FIG. 10 is an example of a flow to form wearable device, according to some embodiments;

FIG. 11 is an exploded view of an example of a wearable pod having, for example, an opaque surface, according to some embodiments;

FIG. 12 is a diagram depicting a touch-sensitive I/O controller, according to some embodiments;

FIGS. 13A to 13D are diagrams depicting various aspects of an interface of a wearable pod, according to some examples;

FIGS. 14A to 14D depict examples of micro-perforations, according to some examples;

FIGS. 15A to 15D are diagrams depicting another example of a display portion for a wearable pod, according to some embodiments;

FIG. 16 is an example of a flow to form a wearable pod, according to some embodiments;

FIG. 17 illustrates an exemplary computing platform disposed in a wearable pod configured to facilitate a touch-sensitive interface in an opaque or predominately opaque surface in accordance with various embodiments;

FIG. 18 is an exploded perspective view of an example of a wearable pod having, for example, a metal surface, according to some embodiments;

FIG. 19 is an exploded front view of an example of a wearable pod having, for example, a metal surface, according to some embodiments;

FIGS. 20A to 20B are respective exploded perspective and exploded front views of a wearable pod including anchor portions, according to some embodiments;

FIG. 20C is a bottom perspective view of a pod cover implementing a sealant during assembly, according to some embodiments;

FIG. 20D is a diagram depicting a perspective front view of a wearable pod being assembled as part of a wearable device, according to some embodiments;

FIGS. 21A and 21B are diagrams depicting a cross-section of a portion of an isolation belt, according to some examples;

FIG. 22 illustrates an example of a flow to form a touch-sensitive pod cover for a wearable pod, according to some examples; and

FIG. 23 illustrates an example of a flow for a touch-sensitive wearable pod, according to some embodiments

FIG. 24 is a diagram depicting an antenna configured for implementation in a wearable pod having a metallized interface, according to some embodiments;

FIGS. 25A to 25C depict examples of an antenna oriented relative to an attachment portion of a cradle, according to some embodiments;

FIG. 26 is an exploded perspective view of an anchor portion, according to some embodiments;

FIG. 27 is an example of a flow to manufacture a communications antenna in a wearable pod and/or device, according to some embodiments;

FIG. 28 is a diagram depicting an antenna configured for implementation in a wearable pod having a metallized interface, according to some embodiments;

FIGS. 29A and 29B are perspective views of an attachment portion and an anchor portion, respectively, according to some embodiments;

FIG. 30 is a diagram depicting another example of a near field communication antenna implemented in a wearable device;

FIG. 31 is an example of a flow to manufacture a short-range communications antenna in a wearable pod and/or device, according to some embodiments.

FIG. 32 illustrates various examples of a wire bus and components coupled with the wire bus, according to some embodiments;+

FIG. 33 illustrates top, side, and bottom plan views of a wire bus, according to some embodiments;

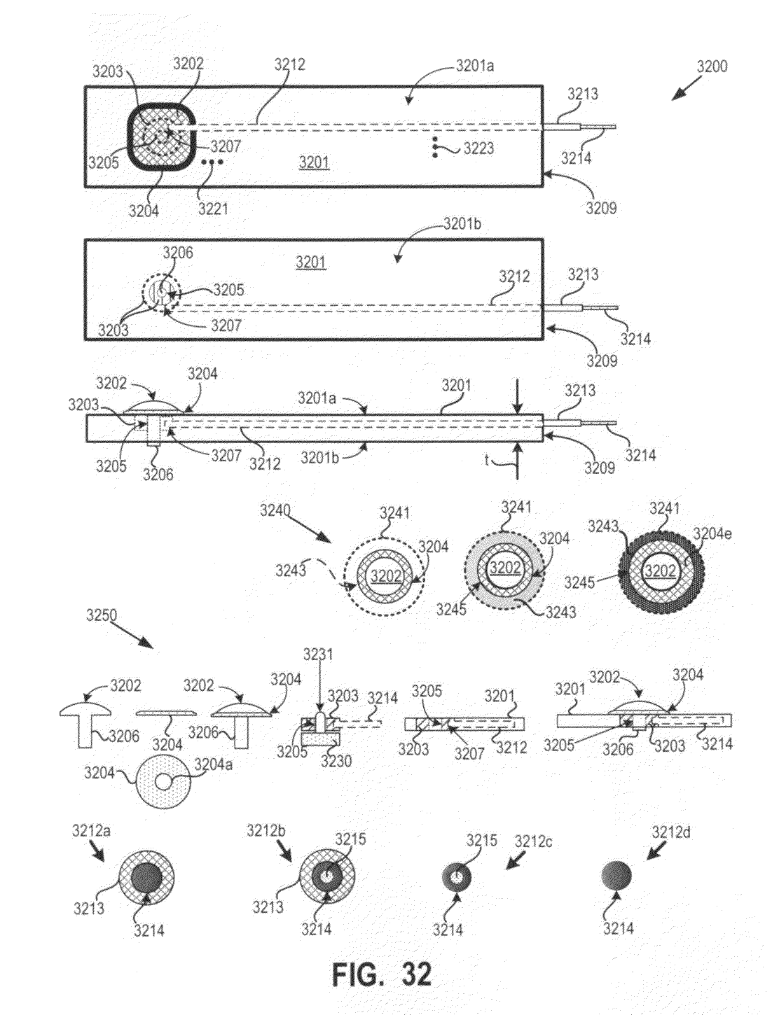

FIG. 34 illustrates one example of a wire bus including a wire bridge, according to some embodiments;

FIG. 35 illustrates one example of a wire bus including a substrate having an antenna coupled with a near field communication chip, according to some embodiments;

FIG. 36 illustrates examples of relative spacing and dimensions of electrodes included in a wire bus, according to some embodiments;

FIG. 37 illustrates examples of wire routing and connection with pads included in a wire bus, according to some embodiments;

FIG. 38 illustrates a side view of one example of an electrode, a skirt, and a pad that may be included in a wire bus, according to some embodiments;

FIG. 39 illustrates profile views of other examples of an electrode, a skirt, and a pad that may be included in a wire bus, according to some embodiments;

FIG. 40 illustrates various views of yet other examples of an electrode, a skirt, and a pad that may be included in a wire bus, according to some embodiments;

FIG. 41 illustrates one example of an assembly order of a strap band that includes a wire bus, an inner strap and an outer strap, according to some embodiments;

FIG. 42 illustrates one example of a wire bus being coupled with an inner strap, according to some embodiments;

FIG. 43 illustrates one example of an outer strap being formed on wire bus coupled with an inner strap, according to some embodiments;

FIG. 44 illustrates top, side and bottom views of one example of a strap band that includes an encapsulated wire bus and sealed electrodes, according to some embodiments;

FIG. 45 illustrates examples of fastening hardware that may be coupled with a strap band, according to some embodiments;

FIG. 46 illustrates one example of a flow diagram for a method of fabricating a wire bus, according to some embodiments;

FIG. 47 illustrates one example of a flow diagram for a method of fabricating a strap band that includes a wire bus, according to some embodiments;

FIG. 48 illustrates various views of a strap band, according to some embodiments;

FIG. 49 illustrates examples of a strap band positioned on a body portion, according to some embodiments;

FIG. 50 illustrates a side view of a strap band coupled with a device, according to some embodiments;

FIG. 51 illustrates a top plan view and a side view of a strap band, according to some embodiments;

FIG. 52 illustrates profile views of a system including a strap band, according to some embodiments;

FIG. 53 illustrates views of a strap band and relative dimensions and positions of components of the strap band, according to some embodiments;

FIG. 54 illustrates a side view and top plan view of a wire bus, according to some embodiments;

FIG. 55 illustrates various examples of electrodes, according to some embodiments;

FIG. 56 illustrates examples of circuitry coupled with electrodes of a strap band, according to some embodiments;

FIG. 57 illustrates profile views of systems that include a strap band, according to some embodiments;

FIG. 58 illustrates exemplary data types for device-based activity classification using predictive feature analysis;

FIG. 59 illustrates an exemplary computing network topology for device-based activity classification using predictive feature analysis;

FIG. 60 illustrates an exemplary application architecture for device-based activity classification using predictive feature analysis;

FIG. 61 illustrates an exemplary process for device-based activity classification using predictive feature analysis;

FIG. 62 illustrates another exemplary process for device-based activity classification using predictive feature analysis;

FIG. 63 illustrates a further exemplary process for device-based activity classification using predictive feature analysis;

FIG. 64 illustrates yet another exemplary process for device-based activity classification using predictive feature analysis; and

FIG. 65 illustrates an exemplary computer system suitable for device-based activity classification using predictive feature analysis.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

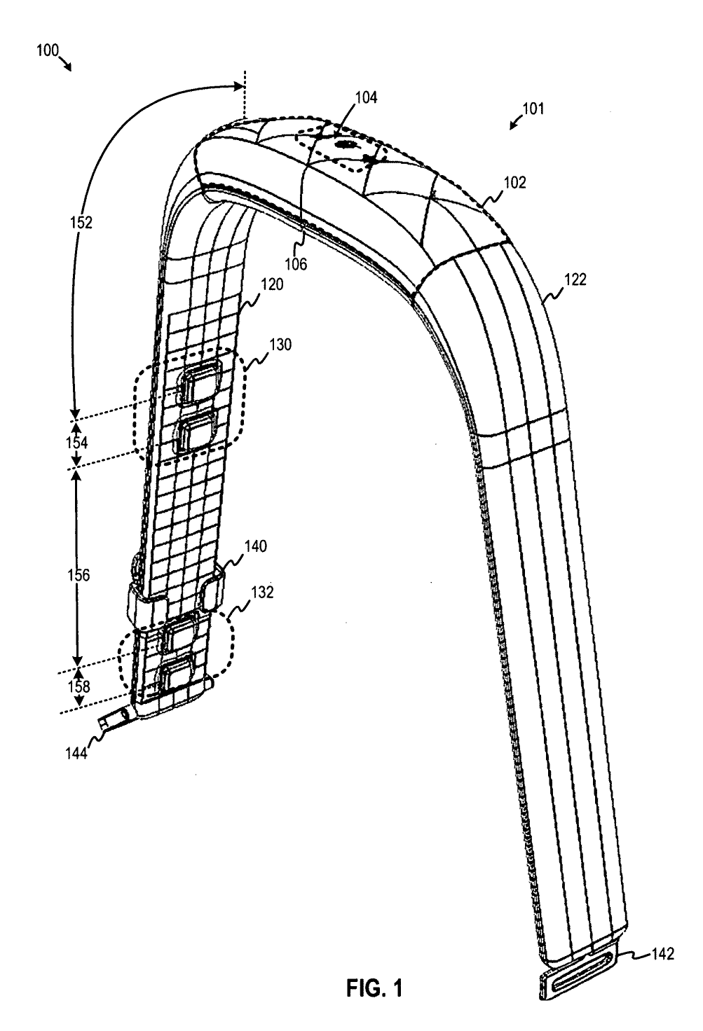

FIG. 1 is a perspective view of a wearable device, according to some embodiments. Diagram 100 illustrates a wearable device including a wearable pod 101 including logic, whether in hardware, software or combination thereof, a strap band 120 and band 122. Among other things, strap band 120 and band 122 are composed of material designed to provide comfort while being worn by a user. In the example shown, the logic is disposed between a top pod cover 102 and a bottom pod cover 106. Top pod cover 102 may be formed in a substrate of an opaque material, such as metal. According to some embodiments, one or more portions of pod cover 102 are configured to accept user input by way of detected capacitance values (or changes in capacitance values), thereby effectuating capacitive touch sensing (e.g., “cap touch”) as a means receiving commands or inputs from a user.

A display portion 104 is disposed at the predominately opaque portion of talk pod cover 102, and is configured to emit light of various shapes (e.g., any type of symbol) and colors to convey information to a user. In one example, display portion 104, or portions thereof, is selectably opaque in that some portions selectable do not emit light while and other arrangements of light to transmit through the surface. As such, display portion 104 may be configured to provide or output information to a user, the information describing aspects of the activity in which users engaged, progress toward a goal of completing the activity, physiological information, such as heart rate, among other things. Further, wearable pod 101 includes any number of sensors and related circuitry, such as bioimpedance circuitry and sensors, galvanic skin response circuitry and sensors, temperature-related circuitry and sensors, and the like.

Strap band 120 includes any number of groups of electrodes. As shown, a group 130 of electrodes is disposed at an approximate distance 152 from wearable pod 101, whereby a first electrode is separated by an approximate distance 154 from the second electrode in group 130. Group 132 of electrodes is shown to be disposed at an approximate distance 156 from group 130, with a first electrode in group 132 being separated at an approximate distance 158 from a second electrode. The approximate distances are configured to dispose one of either group 130 of electrodes or group 132 of electrodes adjacent to a first blood vessel (e.g., an ulnar artery) and to dispose the other group of either group 130 of electrodes or group 132 of electrodes adjacent to a second blood vessel (e.g., a radial artery). Logic in a wearable pod 101 can be coupled to electrodes in groups 130 and 132 to employ bioimpedance sensing for extracting heart-related information, as well as other physiological information, including but not limited to respiration rates. According to some examples, distances 154 and 158 may be about 4.0 mm+/−50%, and distance 156 may range from about 31.5 mm to about 36.0 mm+/−30%, depending on technologies used to pick-up and monitor bioimpedance signals. Distance 152 may be about 32.0 mm+/−30%.

According to some embodiments, one or more electrodes of groups 130 and 132 of electrodes may be configured for multi-mode use. For example, an electrode may be implemented to effect bioimpedance sensing in one mode, and electrode may be used to implement galvanic skin conductance sensing in another mode. In some instances, electrode from group 130 may operate cooperatively with an electrode in group 132. Note that while strap 120 may be described as a “strap band” and strap 122 may be described as a “band,” the terms strap and band may be used, at least in some examples, interchangeably.

In the example shown, the wearable device includes a latch 142, a loop 144, and a latched buckle 140 are configured to engage so as to secure the wearable device around an appendage, such as a wrist. For example, a user may place wearable pod 101 on a top of a wrist, and insert latch 142 through loop 144 adjacent the bottom of the user's wrist, whereby latch 142 engages latched buckle 140. Note while the wearable device is described as being configured to encircle a wrist, and various other embodiments facilitate attachment to any other appendage of the user, including an ankle, neck, ear, etc.

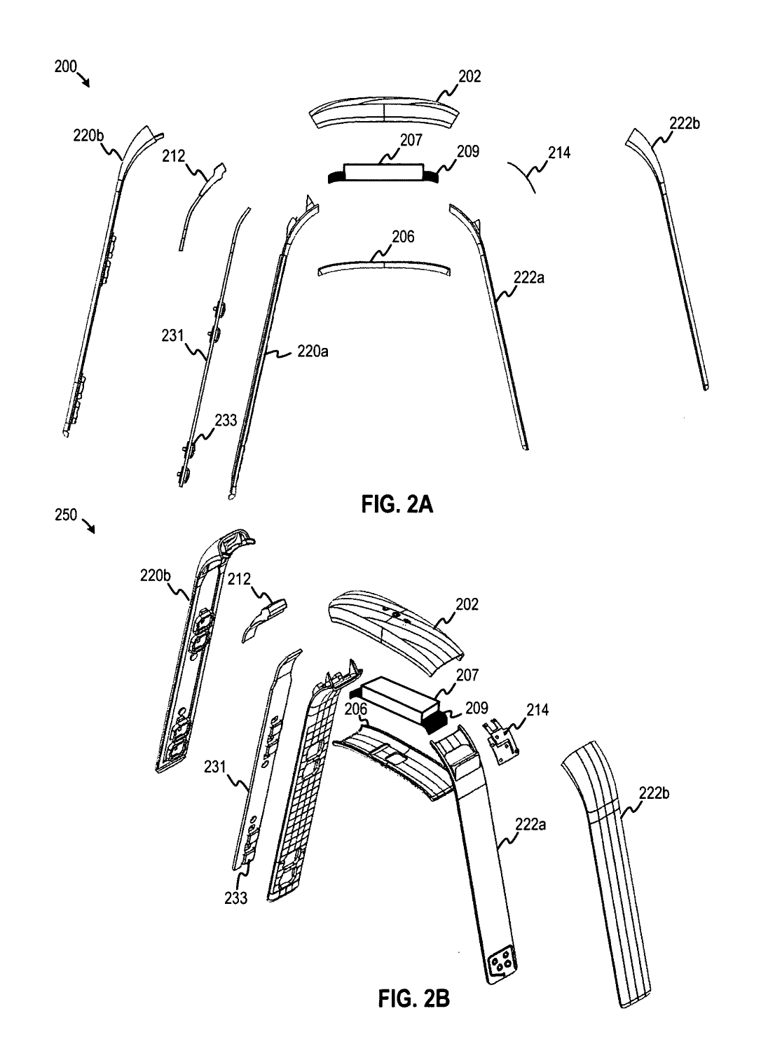

FIGS. 2A and 2B are diagrams depicting an exploded front view and an exploded perspective view, respectively, of a wearable device, according to some embodiments. Diagram 200 of FIG. 2A illustrates a wearable device in an exploded front view, the wearable device including a top pod cover 202 and a bottom pod cover 206 that are configured to enclose an interior region within a cradle 207 having anchor portions 209 that securely couples strap band 220 and band 222 to cradle 207. Strap band 220 is shown to include an inner portion 220a upon which an electrode bus 231 is disposed thereupon. Electrode bus 231 includes electrodes 233 and conductors coupled between electrodes 233 and circuitry within cradle 207. In some embodiments, a near field communications (“NFC”) system 212 can be disposed in contact on electrode bus 231, which may support system 212. Near field communication system 212 may include an antenna to receive/transmit via NFC protocols, and an active near field communication semiconductor device to receive/transmit data. An outer portion 220b is then formed to encapsulate electrode bus 231 and NFC system 212 in portions 220b and 220a to form strap band 220, which is anchored at anchor portion 209 to cradle 207. Band 222 is shown to include an inner portion 222a and an outer portion 222b that encapsulates a short-range antenna 214, such as a Bluetooth® LE antenna, and attaches to cradle 207 at anchor point 209. FIG. 2B is a diagram 250 that illustrates an exploded perspective view of wearable device described in FIG. 2A.

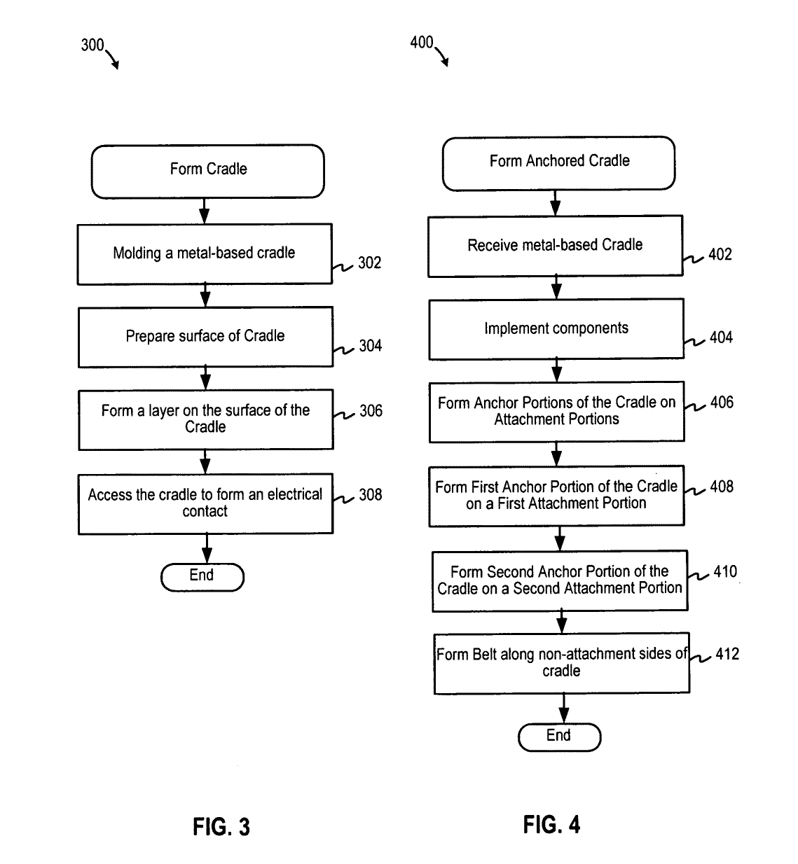

FIGS. 3 and 4 are flow diagrams depicting examples of flows for forming a cradle and forming an anchored cradle, respectively, according to some embodiments. FIG. 3 is a flow 300 to form a cradle where, at 302, a metal-based cradle is molded. According to various examples, a metal injection molding (“MIM”) process may be used to form a cradle that is configured to rigidly house circuitry and to secure a strap band and a band to each other. According to some examples, the cradle can be formed using semi-solid metal (“SSM”) casting techniques. A cradle may also be formed thixomolding processes to form, for example, a magnesium-based cradle (“thixo magnesium cradle”) having sufficient strength and being relatively light-weight, according to some embodiments. At 304, a surface of the cradle is prepared by removing unnecessary material and cleaning the cradle, as an example. At 306, a layer is deposited on the surface of the cradle, such as during electro-deposition. In some cases, the layer is protective (e.g., corrosion resistant) and nonconductive. At 308 the metallic interior is accessed to form electrical contact so that, for example, the cradle can be electrically coupled to ground, which is a common ground for some circuitry and, in some cases, the bottom pod cover.

FIG. 4 is a flow 400 to form an anchored cradle, according to some examples. At 402 a metal-based cradle is received, an example of which is formed by flow 300 of FIG. 3. Further to flow 400 of FIG. 4, some components implemented in a cradle may be set for further processing (e.g., molding). For example, pins, such as pogo pins, can be set prior to molding to prepare formation of a communication port and/or a charging port, which may be implemented as a USB port. As another example, a temperature sensor can be set prior to molding, the temperature sensor configured to extend through the bottom pod cover. At 406, anchor portions of the cradle are formed on the attachment portions. In some embodiments, a first anchor portion of the cradle is formed on a first attachment portion at 408, and a second anchor portion is formed on a second attachment portion of the cradle at 410. Further, an isolation belt may be formed at 412 along sides of the cradle (e.g., the longitudinal sides), the isolation belt being configured to isolate metallic portions of a wearable device from electrically contacting each other. For example, a top pod cover may be electrically isolated from a bottom pod cover or other portions of the wearable device to facilitate capacitive touch sensing. According to some embodiments, the above-described blocks 408, 410, and 412 can be performed in parallel. In one instance, and anchored cradle can be formed in a polycarbonate molding process to form anchor portions and an isolation belt, as well as a layer below the cradle to secure the pins and temperature sensor in place. In some embodiments, one of blocks 408 and 410 can include encapsulating an antenna in an anchor portion, as described herein.

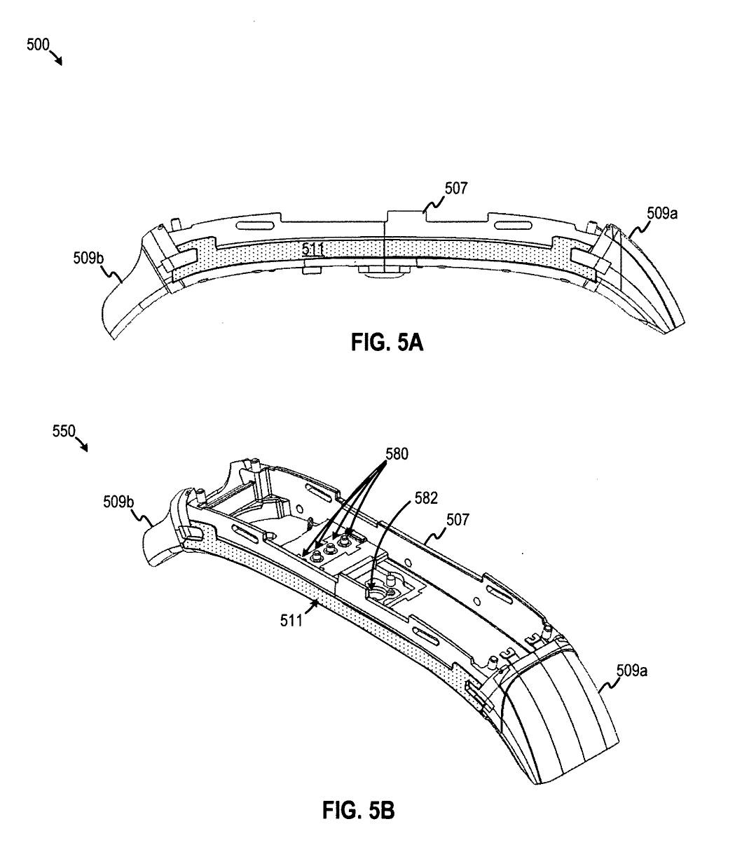

FIGS. 5A and 5B are diagrams depicting a front view and a perspective view, respectively, of an anchored cradle, according to some embodiments. FIG. 5A is a diagram 500 depicting a front view of an anchored cradle 507 having anchor portions 509a and 509b formed at the distal ends of cradle 507. Also shown, and isolation belt 511 formed along a longitudinal side of cradle 507. Anchor portion 509a may include, for example, a Bluetooth® low energy (“LE”) antenna formed therein, and anchor portion 509 be configured to receive an electrode bus and an NFC antenna (and NFC chip). FIG. 5B is a diagram 550 depicting a perspective view of an anchored cradle 507 of FIG. 5A. Further, diagram 550 illustrates pins 580 and a temperature sensor 582 molded and integrated into anchored cradle 507.

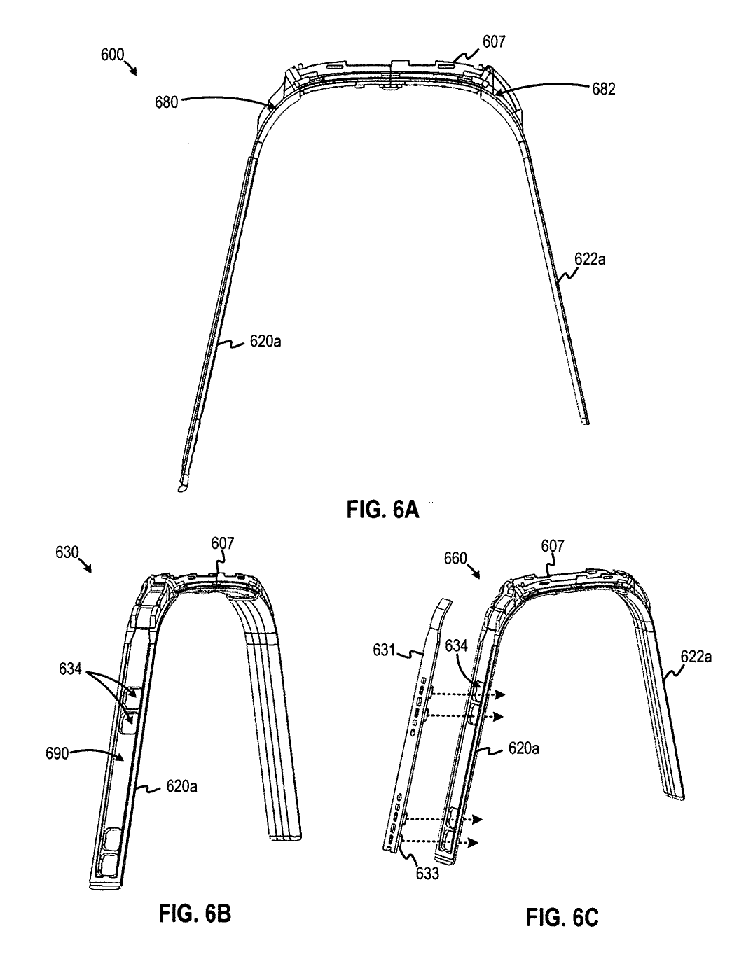

FIGS. 6A to 6C are diagrams depicting formation of an intermediate assembly structure formed in molding process, according to some examples. Consider that an anchored cradle is placed in a mold for forming straps (e.g., strap bands and bands) for a wearable device. Diagram 600 of FIG. 6A illustrates a front view of an anchored cradle 607 integrated with an inner strap portion 620a and an inner strap portion 622a. Inner strap portion 620a is secured to an anchor portion at an interface 680, whereby the interface materials of the anchor portion form relatively secure physical and chemical bonds. Similarly, inner strap portion 622a is secured to the other anchor portion and at an interface 682.

According to some embodiments, the interface materials that form the anchor portions can include, but are not limited to, polycarbonate materials, or other like materials. Polycarbonate may provide an interface to couple metal cradle 607 to an elastomer material used to form inner portions 620a and 622a. Thus, an interface materials, such as polycarbonate, bridges the difficulties of bonding metal and elastomers together in some cases. Anchor portions can be formed using polycarbonate molding techniques. According to some embodiments, an elastomer material may be a thermoplastic elastomer (“TPE”). In one embodiment, elastomer includes a thermoplastic polyurethane (“TPU”) material. In some examples, the elastomer has a hardness in a range of 58 to 72 Shore A. In one case, the lesser has a hardness in a range of 60 to 70 Shore A. An example of an elastomer is a GLS Thermoplasic Elastomer Versaflex™ CE Series CE 3620 by PolyOne of OH, USA.

FIG. 6B is a diagram depicting a perspective view of a strap band, going to some examples. Diagram 630 illustrates a cavity 690 and apertures 634 in inner portion 620a formed by a mold. Apertures 634 can be for receiving electrodes. FIG. 6C is a diagram 660 depicting a perspective view of an assembly of an electrode bus with an inner portion 620a. As shown, electrode bus 631 includes electrodes 633, which are inserted through corresponding apertures 634 prior to a molding step (e.g., a second shot). According to some embodiments, an elastomer material, such as TPE or TPU, may be used to form a flexible substrate in which Kevlar™-based conductors are encapsulated. In one example, the flexible substrate is formed of TPE and has a hardness of approximately 85 to 95 Shore A (e.g., about 90 Shore A).

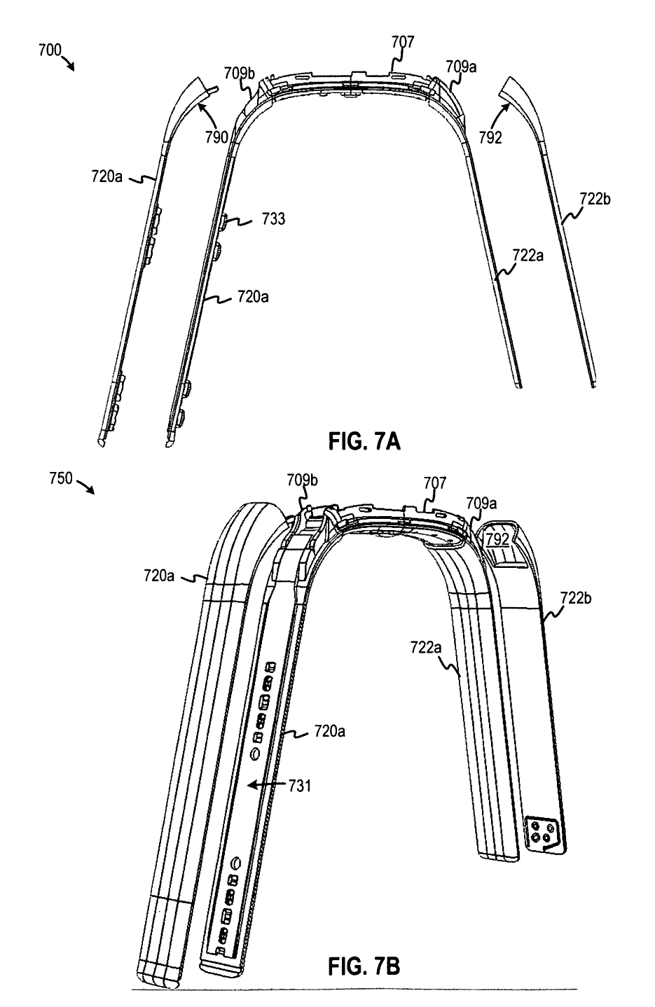

FIGS. 7A to 7B are diagrams depicting formation of another intermediate assembly structure formed in molding process, according to some examples. Diagram 700 of FIG. 7A illustrates formation of outer portion 720a and outer portion 722b in a molding step. In particular, an anchored cradle 707 includes anchored portions 709a and 709b integrated and/or physically coupled to inner portion 722a and inner portion 720a, respectively, subsequent to the molding process described in FIGS. 6A to 6B. Further to FIG. 7A, anchored cradle and inner portions 720a and 722a can be inserted into a mold and material can be injected into the mold to form outer portion 720b over anchor portion 709b and inner portion 720a, and to form outer portion 722b over anchor portion 709a and inner portion 722a. In some embodiments, inner portions 720a and 722a are formed with the same materials as outer portion 720b and 722b. Further, inner surface areas 790 and 792 may be integrated and/or coupled to respective surfaces of anchor portion 709b and 709a to form a secure mechanical coupling between metal cradle 707 and straps 720 and 722. Diagram 750 of FIG. 7B is a perspective view showing formation of outer portions 720a and 722b, whereby surface area 792 of outer portion 722b forms a secure physical and/or chemical bond to an exposed surface of anchor portion 709a.

A manufacturing process, according to some embodiments, includes placing an anchored cradle of FIG. 6A on a fixture for alignment in a mold for receiving a “first shot” of an elastomer to form the inner portions, and the anchored cradle is then transition to receive a “second shot” of elastomer to form the outer portions integrally with the inner portions. Therefore, according to some embodiments, an anchored cradle can be formed in one or two polycarbonate molding steps, with a subsequent formation of a band (including one or more straps) in one or two elastomer molding steps.

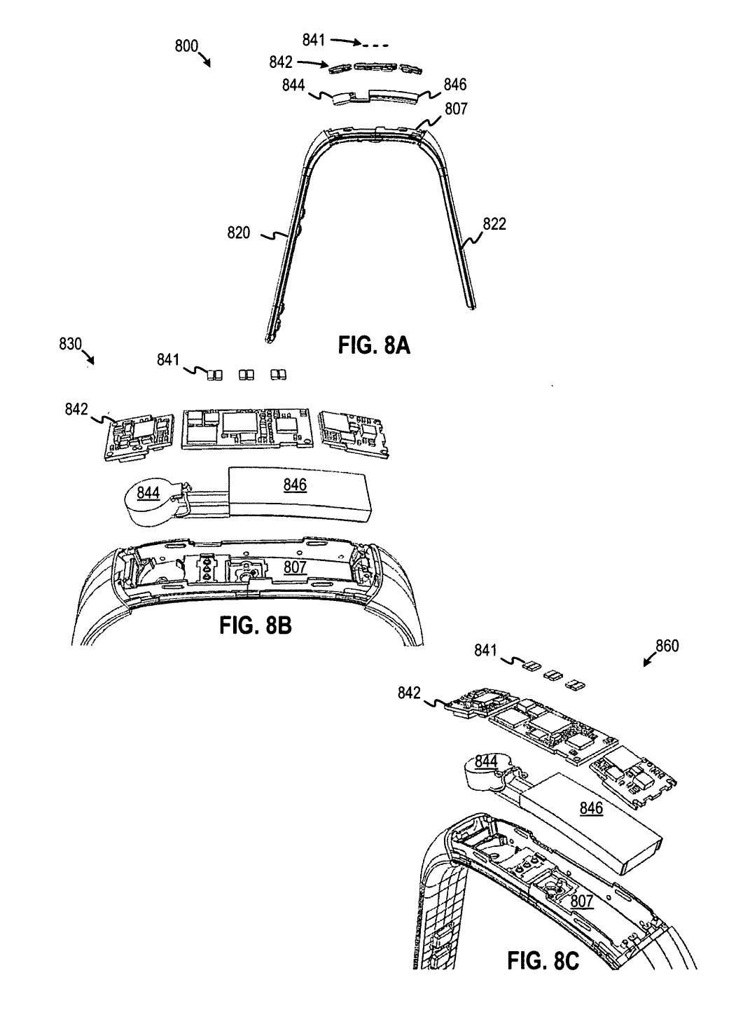

FIGS. 8A to 8C are diagrams depicting exploded views of logic, circuitry, and components disposed within the interior of a cradle anchored to two straps, according to some embodiments. Diagram 800 is an exploded front view depicting a cradle 807 integrated with a strap 820 (or strap band) and a strap 822 (or band). A motor 844, as a source of vibratory energy, and a battery 846 are assembled in the interior of cradle 807. Next, one or more logic modules and/or circuits 842 are disposed over motor 844 and battery 846. Light are positioned above logic modules and/or circuits 842 to emit light through a top pod cover (not shown).

FIG. 8B is a diagram 830 depicting an exploded front perspective view, according some examples. As shown, a vibratory motor 844 and battery 846 are configured to be mounted within the interior of cradle 807. Logic modules and/or circuits 842 are mounted over motor 844 and battery 846 (the mounting hardware is omitted for purposes of clarity). Light sources 841 are oriented above the logic modules and/or circuits 842.

FIG. 8C is an exploded perspective view of the components of FIG. 8B. Logic modules and/or circuits 842 can include a touch-sensitive input/output (“I/O”) controller to detect contact with portions of a pod cover (not shown), a display controller to facilitate emission of light via an opaque or predominately opaque substrate to communicate information to a user, an activity determinator configured to determine an activity based on, for example, sensor data from one or more sensors (e.g., disposed in an interior region between pod covers, or disposed externally thereto). Further, logic modules and/or circuits 842 may include a bioimpedance (“BI”) circuit to use bioimpedance signals to determine a physiological signal (e.g., heart rate), and a galvanic skin response (“GSR”) circuit to use signals to determine skin conductance. Logic modules and/or circuits 842 may include a physiological (“PHY”) signal determinator configured to determine physiological characteristics, such as heart rate, respiration rate, among others, and a temperature circuit configured to receive temperature sensor data to facilitate determination of heat flux or temperature. A physiological (“PHY”) condition determinator implemented in logic modules and/or circuits 842 may be configured to implement heat flux or temperature, or other sensor data, to derive values representative of a condition (e.g., a biological condition, such as caloric energy expended or other calorimetry-related determinations). Other structures, circuits, and/or functions within the scope of the present disclosure.

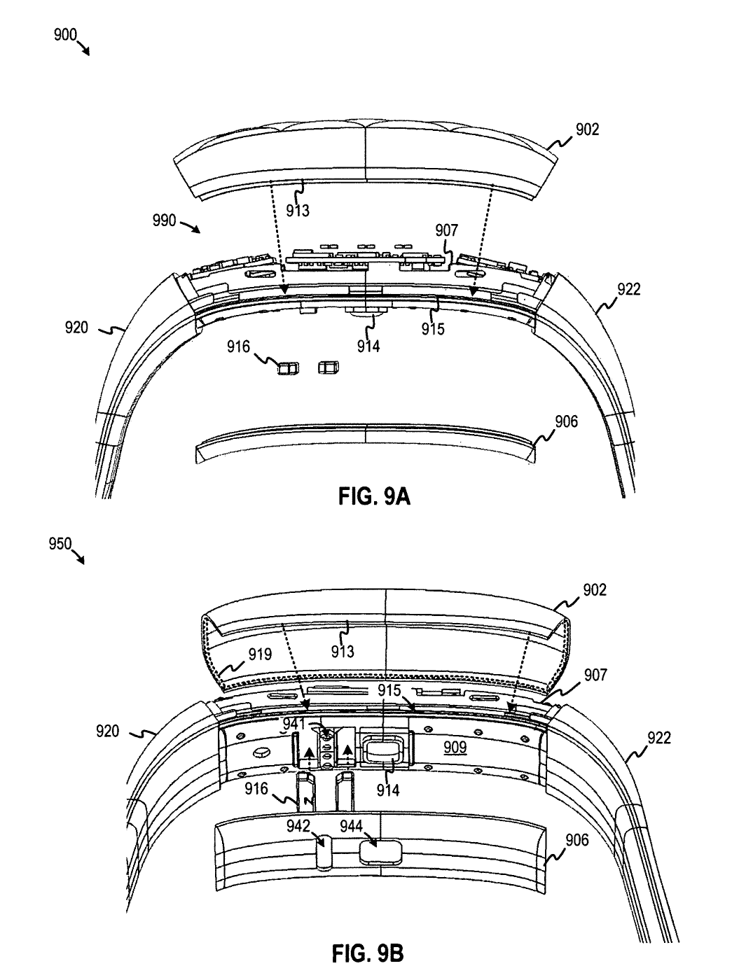

FIGS. 9A and 9B are diagrams depicting an assembly step in which one or more pod covers of a wearable pod are integrated into a wearable device, according to some embodiments. Diagram 900 illustrates a top pod cover 902 oriented for assembly to enclose an interior region 990 of cradle 907 that includes logic, components, circuitry, etc. described, for example, in FIGS. 8A to 8C. At this stage of assembly, straps 920 and 922 are anchored to cradle 907, which includes a temperature sensor 914 configured to protrude external to bottom pod cover 906. Edges 913 of pod cover 902 may include adhesive/epoxy configured to form a fluid-resistant seal as a barrier to prevent fluids (e.g., gas, liquid, moisture, etc.) from entering interior region 990. An isolation belt 915, as shown, is configured to isolate top cover 902 and bottom cover 906. Similarly, edges of pod cover 906 (and other portions) may also include epoxy to couple to form a wearable pod.

FIG. 9B is diagram 950 depicting a bottom perspective view of elements shown in FIG. 9A. Diagram 950 illustrates top cover 902 having epoxy 919 or sealant in the interior of top cover 902 and disposed at or near edges 913. A wired communications port includes a number of pins 941 (e.g., a USB port) disposed adjacent to magnets 916 mounted in cavities within the bottom 909 of cradle 907. Magnets 916 are configured to form a magnetic attachment to a corresponding connector that can provide power, ground, and data signals via aperture 942 of bottom pod cover 906. Also shown in FIG. 9B is a temperature sensor 914 that extends through temperature 944 to contact skin of a user.

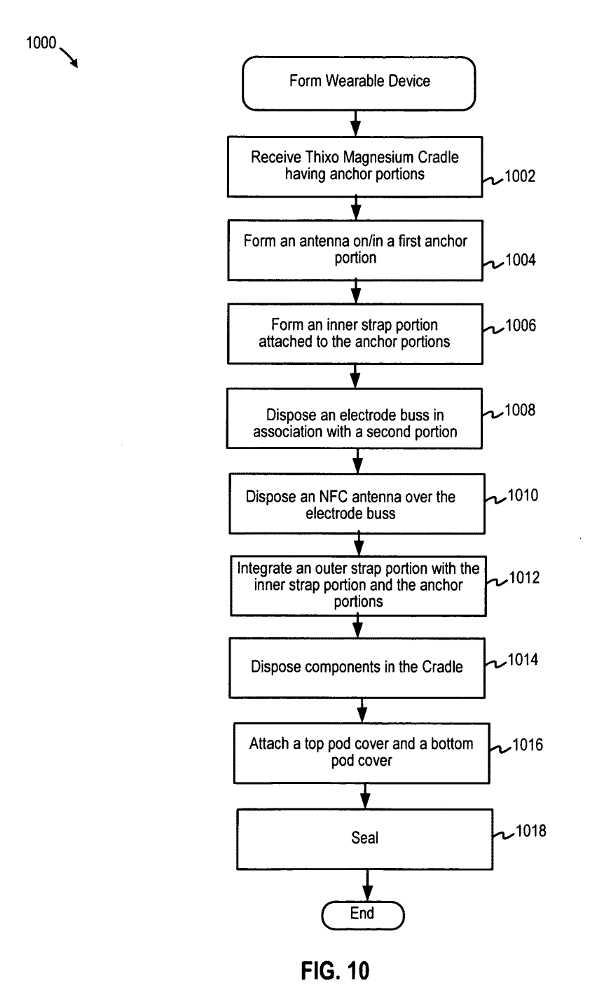

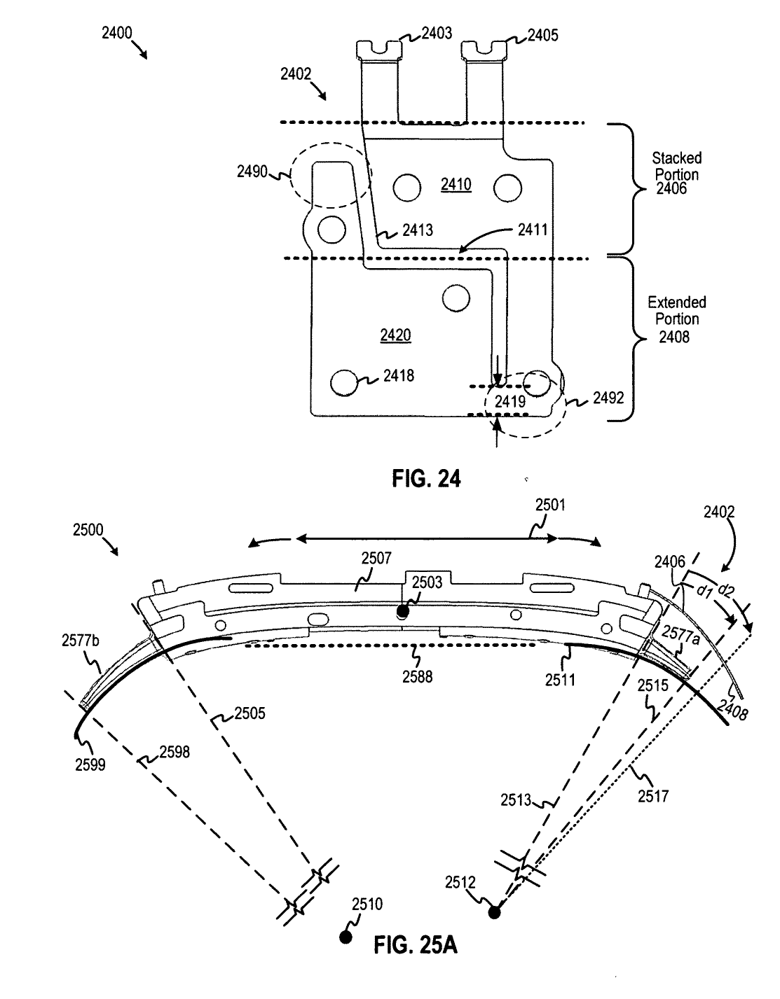

FIG. 10 is an example of a flow to form wearable device, according to some embodiments. Flow 1000 includes receiving a fix so magnesium cradle having anchor points at 1002, and forming an antenna in a first anchor portion at 1004. An example of an antenna being formed in a portion is described in, for instance, FIG. 26. At 1006, and neuter strap portion is formed attached to the anchor portions. At 1008, an electrode bus can be disposed within the inner portion of the strap band, and an NFC antenna can be disposed over the electrode bus at 1010. At 1012, an outer strap portion is integrated with the inner strap portion and is further integrated with, or attached to, the anchor portions. At 1014 components including logic, sensors, circuitry, etc. are disposed in a cradle, and pod covers are attached at 1016. The pod covers are sealed at 1018 to form a fluid-resistant barrier for a wearable pod or device.

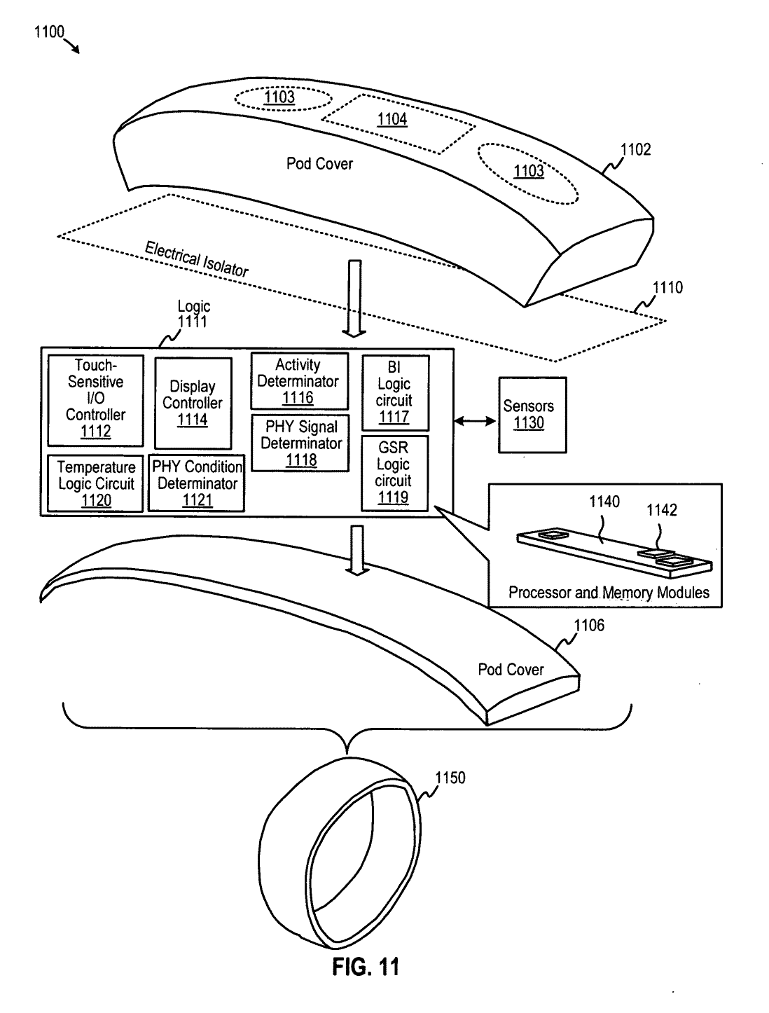

FIG. 11 is an exploded view of an example of a wearable pod having, for example, an opaque surface, according to some embodiments. Diagram 1100 illustrates a pod cover 1102 and a pod cover 1106 configured to house circuitry 1142 including one or more substrates 1140 (e.g., printed circuit board, such as a flex circuit board) and any number of associated processor modules, semiconductor devices (e.g., sensors, radio frequency or “RF” transceivers, etc.), electronic components (e.g., capacitors, resistors, sensors, etc.), and memory modules. Diagram 1100 illustrates the structure and/or functionality of circuitry 1142 as logic 1111. According to some embodiments, pod cover 1102 is shown to include touch-sensitive portions 1103 and a display portion 1104 disposed in a top surface 1102a that predominantly includes an opaque material, such as a metal, a nontransparent plastic, etc. Note that touch-sensitive portions of pod cover 1102 need not be limited to portions 1103. For example in some examples, display portion 1104 may also be configured to function as touch-sensitive portion 1103. As another example, one or more sides and/or surfaces of pod cover 1102 can be implemented as a touch-sensitive portion. An electrical isolator 1110 is shown in diagram 1100, whereby electrical isolator 1110 is configured to electrically isolate touch-sensitive portions 1103 from logic 1111, pod cover 1106, and other components or elements of a wearable pod. In some examples, isolator 1110 can electrically isolate pod cover 1102 and its constituent materials from logic 1111, pod cover 1106, and other components or elements of a wearable pod.

According to some embodiments, pod cover 1102, logic 1111, and pod cover 1106 can be assembled to form a wearable pod that can be integrated into a band 1150 of one or more attachment members (e.g., one or more straps, etc.) to form a wearable device. A wearable pod and/or wearable device may be implemented as data-mining and/or analytic device that may be worn as a strap or band around or attached to an arm, leg, ear, ankle, or other bodily appendage or feature. In other examples, a wearable pod and/or wearable device may be carried, or attached directly or indirectly to other items, organic or inorganic, animate, or static. Note, too, that wearable pod enough be integrated into band 1150 and can be shaped other than as shown in FIG. 11 for example, a wearable pod circular or disk-like in shape with display portion 1104 disposed on one of the circular surfaces.

According some embodiments, logic 1111 includes a number of components formed in either hardware or software, or a combination thereof, to provide structure and/or functionality for elemental blocks shown. In particular, logic 1111 includes a touch-sensitive input/output (“I/O”) controller 1112 to detect contact with portions of pod cover 1102, a display controller 1114 to facilitate emission of light, an activity determinator 1116 configured to determine an activity based on, for example, sensor data from one or more sensors 1130 (e.g., disposed in an interior region between pod covers 1102 and 1106, or disposed externally). A bioimpedance (“BI”) circuit 1117 may facilitate the use of bioimpedance signals to determine a physiological signal (e.g., heart rate), and a galvanic skin response (“GSR”) circuit 1119 may facilitate the use of signals representing skin conductance. A physiological (“PHY”) signal determinator 1118 may be configured to determine physiological characteristic, such as heart rate, among others, and a temperature circuit 1120 may be configured to receive temperature sensor data to facilitate determination of heat flux or temperature. A physiological (“PHY”) condition determinator 1121 may be configured to implement heat flux or temperature, or other sensor data, to derive values representative of a condition (e.g., a biological condition, such as caloric energy expended or other calorimetry-related determinations). Logic 1111 can include a variety of other sensors, some which are described herein, and others that can be adapted for use in the structures described herein.

Touch-sensitive portions 1103 are configured to detect contact by an item or entity as an input to logic 1111. According to some embodiments, touch-sensitive portions 1103 are coupled to touch-sensitive input/output (“I/O”) controller 1112, which is configured to detect a capacitance value at one or more touch-sensitive portions 1103. Further, touch-sensitive I/O controller 1112 can be configured to detect a change from one value of capacitance relative to a touch-sensitive portion 1103 to another value of capacitance. If the value of capacitance is within a range of capacitive values that define a contact as a valid “touch,” touch-sensitive I/O controller 1112 can generate a signal including data describing touch-related characteristics of the contact. Examples of a range of capacitance values include approximate values of 0.75 pF to 2.4 pF, or other equivalent values. Further, examples of items or entities for which a “touch” is detected can include tissue (e.g., a finger), a capacitive stylus (or the like), etc. Touch-related characteristics, for example, can include a number of touches per unit time, a time interval during which a touch is detected, a pattern of different durations per unit time (e.g., such as Morse code or other simplified schemes).

While touch-related characteristics may be a function of time, various implementations need not so limited. For example, consider an implementation of pod cover 1102 with multiple touch-sensitive portions 1103. Touch-related characteristics in this case may also include an order of touching touch-sensitive portions 1103 to simulate, for instance, a swiping gesture from left-to-right or right-to-left. Other types-related characteristics are possible.

Display controller 1114 is configured to receive signals indicative of, for example, a mode of operation of a wearable pod, a value associated with a physiological signal (e.g., a heart rate), a value associated with an activity (e.g., a number of steps, a percentage of completion for a goal, etc.), and other similar information. Further, display controller 1114 is configured to cause selective emission of light via display portion 1104, the emission of light having certain characteristics, such as symbol shapes and colors, to convey specific information.

Bioimpedance circuit 1117 includes logic in hardware and/or software to apply and receive electrical signals include bioimpedance-related information, which physiological signal determinator 1118 can receive and determine one or more physiological characteristics. For example, physiological signal determinator 1118 can extract a heart rate and/or a respiration rate from one or more bioimpedance signals. One or more examples implementing bioimpedance signals to derive physiological signal values are described in U.S. patent application Ser. No. 13/831,260 filed on Mar. 14, 2013, U.S. patent application Ser. No. 13/802,305 filed on Mar. 13, 2013, and U.S. patent application Ser. No. 13/802,319 filed on Mar. 13, 2013, all of which are incorporated by reference herein. A galvanic skin response circuit 1119 includes logic in hardware and/or software to apply and receive electrical signals that includes skin conductance-related information. According to some embodiments, logic 1111 is configured to use electrodes in a first mode to determine bioimpedance signals, and to use at least one for the electrodes in a second mode to determine galvanic skin conductance. Therefore, one or more electrodes may have multiple functions or purposes. Temperature circuit 1120 includes logic in hardware and/or software to apply and receive electrical signals that includes thermal energy-related information, which, for example, physiological condition determinator 1121 can use to derive values representative of a condition of a user, such as a caloric burn rate, among other things.

Examples of other sensors 1130 include accelerometer(s), an altimeter/barometer, a light/infrared (“IR”) sensor, an audio sensor (e.g., microphone, transducer, or others), a pedometer, a velocimeter, a GPS receiver, a location-based service sensor (e.g., sensor for determining location within a cellular or micro-cellular network, which may or may not use GPS or other satellite constellations for fixing a position), a motion detection sensor, an environmental sensor, a chemical sensor, an electrical sensor, a mechanical sensor, a light sensor, and others.

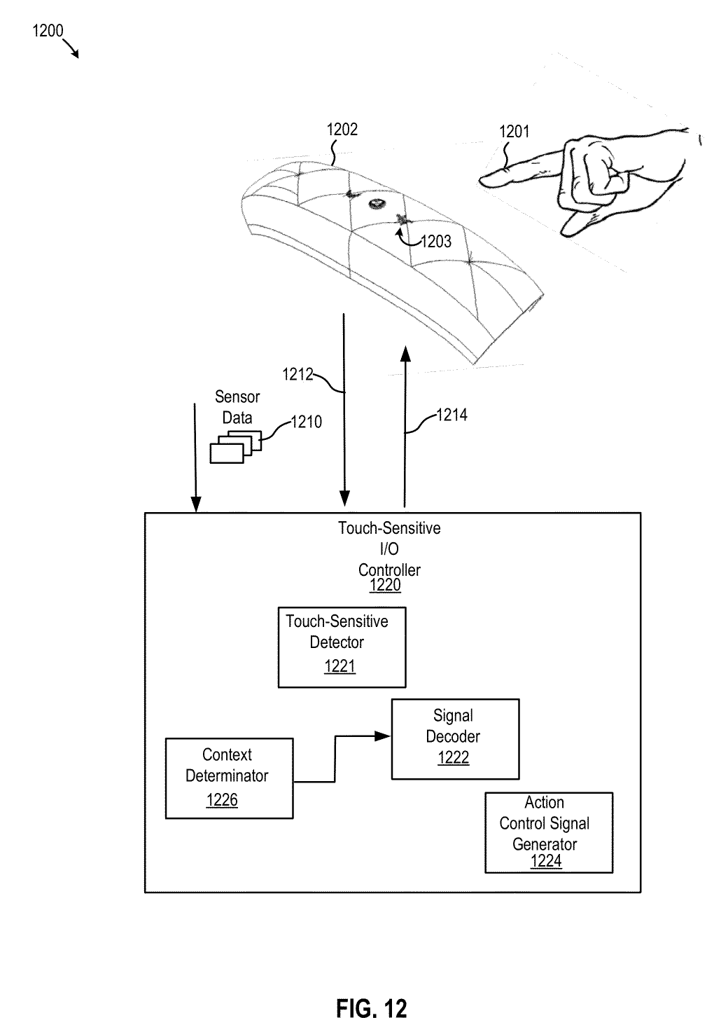

FIG. 12 is a diagram depicting a touch-sensitive I/O controller, according to some embodiments. Diagram 1200 illustrates a touch-sensitive I/O controller 1220 including a touch-sensitive detector 1221, a signal decoder 1222, an action control signal generator 1224 and a context determinator 1226. According to some embodiments, touch-sensitive detector 1221 is coupled to a surface of a pod cover 1202 and is configured to receive one or more signals via a conductive path 1212, the one or more signals indicating a value of detected capacitance. A detected capacitance value can be determined responsive to contact by tissue (e.g., finger 1201) with a portion of pod cover 1202. Touch-sensitive detector 1221 can also be coupled to pod cover 1202 to detect a capacitive value based on contact in a display portion 1203. In some examples, a surface of a pod cover 1202 can include to a surface portion of a substrate, such as a metal substrate, regardless of whether pod cover 1202 is covered in a coating (e.g., anodized or the like).

Signal decoder 1222 is configured to receive one or more signals to decode or otherwise determine a command based on one or more detected capacitance values, according to some examples. As an example, signal decoder 1222 may decode an enable command to enable decoding of one or more detected capacitance signals, thereby enabling a wearable pod to acquire user input via touch. Or, signal decoder 1222 may decode a disable command to disable decoding of one or more signals detected capacitive signals, thereby preventing inadvertent contact (e.g., during sleep, etc.) from being interpreted as being a valid touch. Further, signal decoder 1222 is further configured to decode a number of detected capacitive values to identify patterns of the detected capacitance values, whereby signal decoder 1222 can decode a pattern of detected capacitance values as a specific command. Signal decoder 1222 can determine a pattern of detected capacitance values based on, for example, a quantity of detected capacitance values per unit time, a time interval during which a detected capacitance value is detected, a pattern of varied durations per unit time and/or different detected capacitance values, etc. Thus, signal decoder 1222 can decode detected capacitance values to determine a command as a function of time.

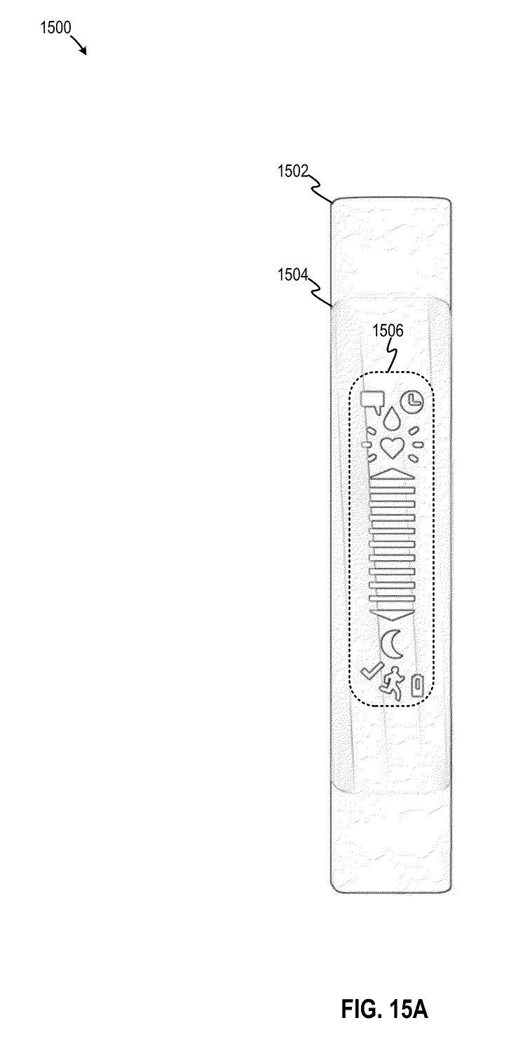

Further to the above-described examples, signal decoder 1222 can identify a first pattern of detected capacitance values associated with a first command to, for example, disable implementation of a subset of subsequent detected capacitance values, thereby disabling implementation by a wearable pod of subsequent detected capacitance values (e.g., turning “off” a ‘cap touch’ input feature to exclude inadvertent touches). Signal decoder 1222 can identify a second pattern of detected capacitance values associated with a second command (e.g., a mode command) to, for example, transition the wearable pod to a mode of operation as a function of a capacitance pattern. Also, signal decoder 1222 can transmit a signal indicating a mode command to action control signal generator 1224, which can directly or indirectly effectuate a change in mode of operation. Or, in some other examples, a mode controller of FIG. 15B can be implemented to cause a change in mode. In some embodiments, action control signal generator 1224 can cause, directly or indirectly, a particular pattern of the light 1214 to be emitted via display 1203 based on the decoded command.

Context detector 1226, which is optional, may be configured to receive sensor data 1210 and/or data indicating a state of activity (e.g., whether an activity is running, sleeping, or the like). Based on sensor data 1210 and/or activity state data, context detector 1226 can detect context of the wearable pod (e.g., a type of activity in which as user is engaged). Context detector 1226 can transmit context data to signal decoder 1222, which, in turn, can be configured to implement a first set of commands based on one pattern of capacitance values based on a first context (e.g., a person is sleeping), and is further configured to implement a second set of commands based on the identical pattern of detected capacitance value based on a second context (e.g., a person is moving). Thus, context detector 1226 can enable a wearable pod to generate different commands using the same pattern of detected capacitance values based on different contexts.

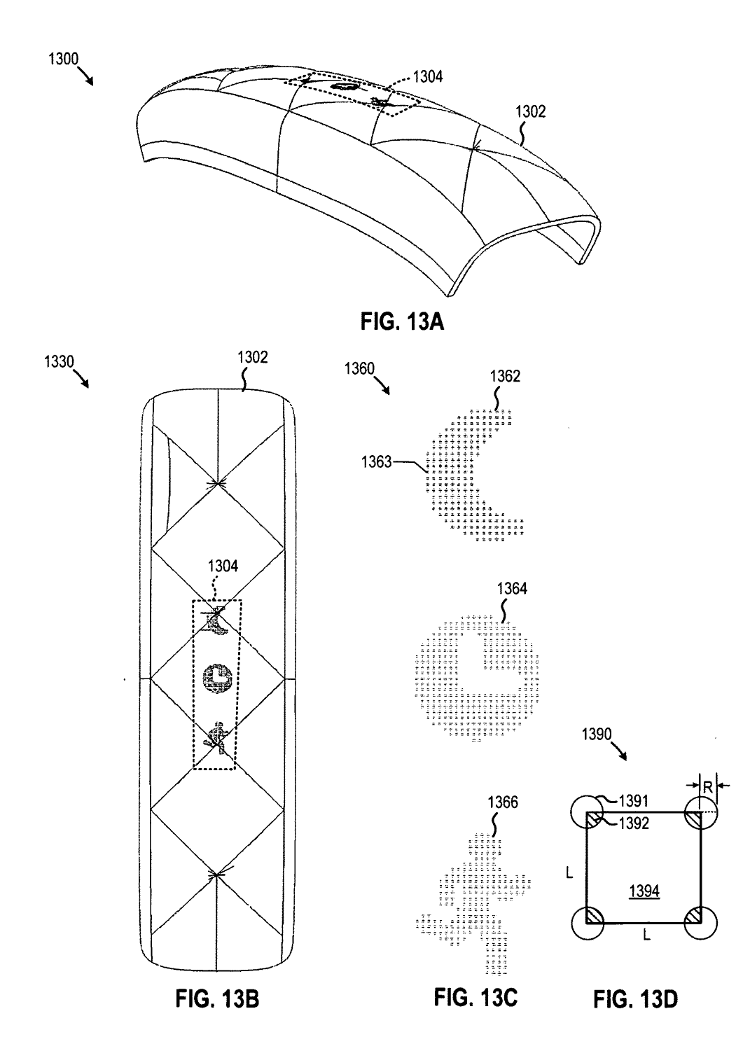

FIGS. 13A to 13D are diagrams depicting various aspects of an interface of a wearable pod, according to some examples. FIG. 13A is a diagram 1300 depicting a perspective view of a pod cover 1302 including a display portion 1304 of an interface. As an interface of a wearable pod, an interface can include a portion of pod cover 1302 that is configured to either accept user inputs or provide an output to a user, or both. Therefore, display portion 1304 can be configured to both output information to a user and accept user input. According to some embodiments, pod cover 1302 includes a conductive material, such as metal, to facilitate touch-sensitive interfacing with a wearable pod. As shown, pod cover 1302 has an elongated shape and includes at least a top surface into side surfaces, all of which are configured to form an interior region into which interior components, such as circuitry, can be disposed. Note that various other embodiments, pod cover 1302 can be formed of any shape including, for example, a circular-shaped cover. In some cases, pod cover 1302 can include a surface treatment (e.g., stamped pattern) including cosmetically-pleasing features.

FIG. 13B is a diagram 1330 depicting a top view of pod cover 1302 including display portion 1304. According to some examples, display portion 1304 includes pixelated symbols formed in an opaque material, such as a metal, a nontransparent plastic, etc. Further, the pixelated symbols may be formed in material to form a predominately opaque material. Other portions of pod cover 1302 can also be formed in an opaque material.

FIG. 13C is a diagram 1360 depicting an enhanced view of display portion 1304. As shown, a display portion can include pixelated symbol 1362 representing a crescent moon (e.g., related to sleep activities and characteristics), pixelated symbol 1364 representing a clock (e.g., related to reminders or information regarding various things, such as sleep activities and workout activities), and pixelated symbol 1366 representing a running person (e.g., related to movement-related activities and characteristics). Further to FIG. 13C, pixelated symbols 1362, 1364, and 1366 are shown to include arrangements of symbol elements 1363. According to some embodiments, a symbol element 1363 may include a micro-perforation. Thus, pixelated symbols 1362, 1364, and 1366 may include arrangements of micro-perforations and/or emissions of light therefrom. The micro-perforations facilitate a display implementing an opaque material or predominately opaque material, whereby a micro-perforation is difficult to see, or is otherwise not visible to most individuals without magnifying equipment.

FIG. 13D is a diagram 1390 that illustrates an example of a density of micro-perforations per unit area in a predominately opaque material. As shown, a unit surface area 1394 of an opaque material, such as anodized aluminum, is shown to include four (4) quarters 1392 of micro-perforation. Area 1394 can be defined by the product of the side lengths, L, whereas the area 1392 is one-fourth (¼) an area defined by a circular (in this example) having a radius, R. In one example, micro-perforations 1391 have diameters of 30 microns (e.g., 0.03 mm) and L is 100 microns (e.g., 0.10 mm). Thus, micro-perforations 1391 in this example may account for about 7% of unit area 1394, and the opaque material is approximately 93% of unit area 1394. With these dimensions, the density of micro-perforations is approximately 100 micro-perforations per square millimeter. Other micro-perforation sizes and densities may be implemented.

According to one example, a predominately opaque material as a portion of a surface can be composed of about 93% opaque material and 7% transparent material per unit area. In another example, a predominately opaque material as a portion of a surface can be composed of about 85% to 98% opaque material per unit area (e.g., approximately 16 to 44 microns), whereas in other examples a predominately opaque material can be composed of about 67% to 99% unit area. In at least one example, a predominately opaque material can be composed of 51% opaque material per unit area. Accordingly, the diameters of micro-perforations 1391 can vary so long as the area consumed by micro-perforations 1391 do not, for example, consume more than 49% of an opaque material. Note while micro-perforations 1391 are depicted as being circular, the size and shape of micro-perforations 1391 are not so limited.

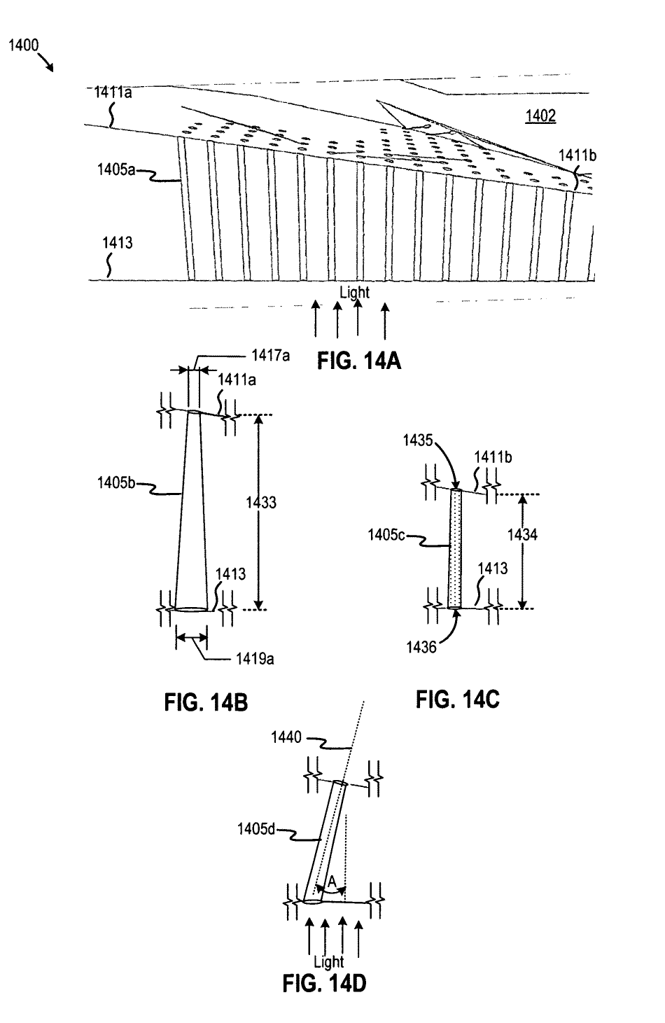

FIGS. 14A to 14D depict examples of micro-perforations, according to some examples. FIG. 14A is a diagram 1400 depicting a cross-section of a pod cover 1402 and micro-perforations 1405a extending from an outer surface 1411a, 1411b to an inner surface 1413, which is adjacent to light sources (not shown) that transmit light for emission via micro-perforations 1405a. FIG. 14B illustrates an example of a tapered micro-perforation, according to some examples. Tapered micro-perforation 1405b is configured to include an opening having a diameter or size 1419a in inner surface 1413, whereas another opening may have a diameter or size 1417a in outer surface 1411a. As shown, diameter 1417a is less than diameter 1419a. According to some embodiments, the ratio of diameter 1419a to diameter 1417a can vary based on the depth 1433 of micro-perforation 1405b. In one example, the ratio can be larger as the depth 1433 increases. In another example, the differences in diameters 1417a and 1419b can vary by +/−10 microns. A larger-size diameter 1419a can increase collection of light or scattered light rays from a light source such as one or more LEDs.

FIG. 14C illustrates an example of another tapered micro-perforation 1405c. In this example, micro-perforation 1405c has an opening in inner surface 1413 having a diameter 1436 and another opening an outer surface 1411b having a diameter 1435. In one example, size of diameter 1436 may be slightly larger than diameter 1435 as a function of depth 1434, which is less than depth 1433 of FIG. 14B. An example of one of depths 1433 and 1434 is approximately 300 microns, and can vary by 50% (or greater in some cases). Or, in some examples diameters 1435 and 1436 are equivalent. The shading of micro-perforation 1405c may depict optically-transparent material disposed therein. In some examples, the optically-transparent material may be an optical adhesive, epoxy resin, or sealant having relatively high refractive indices ranging from 1.50 to 1.56, or higher. For example, the refractive index may range from 1.57 to 1.60, or greater. Rather, the optically-transparent material or filler disposed in micro-perforation 1405c may be configured to transmit 95% visible light (e.g., for sidewall areas determined by a diameter of a micro-perforation). The epoxy or filler material may prevent humidity and other environmental factors from affecting internal LEDs (or the like) and/or circuitry. FIG. 14D illustrates an example of an angled micro-perforation, according to some embodiments. As shown, micro-perforation 1405 is formed to focus emission of light along at line 1440 at an angle “A,” to focus light in a direction a user's eyes most likely are positioned. In this configuration, angle A places line 1440 non-orthogonal to the initial direction of emission from below an inner surface of pod cover 1402. Angle A thereby assists in directing luminosity toward a user and reduces the visibility of such information to other persons' eyes at other positions.

FIGS. 15A to 15D are diagrams depicting another example of a display portion for a wearable pod, according to some embodiments. Diagram 1500 illustrates a wearable pod including a pod cover 1504 integrated or otherwise coupled (e.g., detachably coupled) to a band 1502 or strap 1502 to form a wearable device. In this example, display portion 1506 includes a variety of symbols having multiple functions to convey multiple types of information based on a mode of operation, a type of activity, a contacts, etc. Display portion 1506 can include symbol elements composed of micro-perforations. Further, the symbol elements may emit different colors of light based on the types of information being conveyed.

FIG. 15B is a diagram depicting another display portion interacting with a display controller, according to some examples. Diagram 1520 illustrates a display portion 1521 that includes a display formed in predominately opaque material, whereby the symbol elements formed therein may include various arrangements of micro-perforations. Display controller 1540 includes either hardware or software, or a combination thereof, to implement an alert display controller 1542, a message display controller 1543, a heart rate display controller 1544, an activity display controller 1545, and a notification display controller 1546. Further, display controller 1540 can be coupled to a mode controller 1541, which is configured to provide mode data to display controller 1540. The mode data can describe a mode of operation, a context, an activity, or a condition in which a wearable pod is operating. Responsive to the mode data, display controller 1540 can implement one or more of the above-described controllers 1542 to 1546 to provide mode-specific via display portion 1521. As an example, display controller 1540 can identify a subset of light sources and/or micro-perforations to emit light through an arrangement of micro-perforations constituting one or more symbols indicative of a value of a physiological signal, such as a heart rate.

Alert display controller 1542 is configured to implement symbols 1522, 1524, and 1526 to provide alerts to a user. Upon detecting a notification to check an application residing, for example, on a mobile computing device, alert display controller 1542 may be configured to cause symbol 1522 to emit light. Note that according to some embodiments, an illuminated symbol 1522 can alert a user to the availability of an insight. The term “insight” can refer to, for example, data correlated among a state of user (e.g., number of steps taken, number of our slapped, etc.) and other sets of data representing trends, patterns, and correlations to goals of a user (e.g., a target value of a number of steps per day) and/or supersets of generalized (e.g., average values) of anonymized data for a population at-large. With insight data, the user can understand how an activity (e.g., running, etc.) can affect other aspects of health (e.g., amount of sleep as a parameter). In some embodiments, insight data can include feedback information. For example, insights can include data derived by the structures and/or functions set forth in U.S. Pat. No. 8,446,275, which is herein incorporated by reference to illustrate at least some examples.

Should a reminder or notification arise that requires a user to hydrate or consume water, alert display controller 1542 is configured to cause symbol 1526 to illuminate. Alert display controller 1542 is configured to maintain calendared events and times, and is further configured to receive reminders from another computing device, such as a mobile phone. When emitting light, symbol 1524 may alert a user as a reminder to undertake one of variety of actions based on time or a calendar event. Further, symbol 524 may illuminate with different colors and/or with other symbols in display portion 1521 to indicate one or more of a sleep reminder, a workout reminder, a meal reminder, a custom reminder, and the like.

Message display controller 1543 is configured to convey a message via display portion 1521. While symbols 1528 and 1530 can have multiple functionalities, the following descriptions are in the context of conveying messages. For example, message display controller 1543 can cause symbol 1528 to emit light responsive to detecting that the wearable pod and/or a mobile computing device has received, or is receiving, a message of encouragement (electronic “dopamine”) from a friend or family regarding a user's state or activity. Message display controller 1543 is configured to detect that a friend or family member has communicated a “love tap” (e.g., a gesture, like a squeeze or tap of a wearable pod in the other's possession). To convey the love tap, message display controller 1543 is configured to cause symbol 1530 and symbols 1528 to emit light.

Heart rate display controller 1544 is configured to receive physiological signal information based on one or more sensors. For example, the physiological signal information can specify a heart rate related to, for example, a particular mode of operation (e.g., at rest, asleep, moving, running, walking, etc.). Upon receiving data representing a heart rate, heart rate display controller 1544 can select symbols 1530, 1532, 1535 in one or more of symbols 1533 to convey heart rate information. In some cases, symbol 1534 indicates a minimum heart rate and symbol 1532 indicates a maximum heart rate. In this context, symbol 1530 may indicate a heart rate measurement is being performed or has been performed.

Activity display controller 1545 is configured to receive motion or movement-related signal information based on one or more sensors. For example, the motion data can specify a number of motion units (e.g., steps) relative to a goal of total motion units, or the motion data can specify percentage of completion of a user's activity goal (e.g., a number of steps per day). As such, activity display controller 1545 is configured to select a number of symbols 1533 to specify an amount of progress is being made to a goal. Also, activity display controller 1544 can select either symbol 1536 to specify progress toward a sleep goal or symbol 1538 to specify progress to a movement goal.

Notification display controller 1546 is configured to receive data representing a power level of a battery supplying power to a wearable pod. Based on an amount of charge stored in the battery, the notification display controller 1546 can cause symbol 1539 to emit light to indicate a charge level. Notification display controller 1546 is also configured to receive data representing an indication that a user's action either regarding a wearable pod or a mobile computing device (e.g., an application) has been implemented. To confirm implementation, the notification display controller 1546 is configured to emit light via symbol 1537.

FIG. 15C is a diagram depicting an example of an activity display controller interacting with a display portion, according to some examples. Diagram 1550 illustrates a display portion 1551 coupled to an activity display controller 1545. Activity display controller 1545 can receive data originating as accelerometer signals indicative of an activity, and can determine a value indicative of an activity (e.g., an amount of steps toward a goal). Activity display controller 1545 can also determine whether sleep-related information is to be displayed or whether movement-related information as to be displayed, and can identify a quantity of lights from which to emit light, the quantity of lights being proportional to a value indicative of an activity. As shown, activity display controller 1545 is configured to convey information related to a movement-related activity, and thus causes symbol 1556 to illuminate (i.e., shown as shaded). Activity display controller 1545 is configured to determine a user's progress relative to a goal and selects a subset of symbols from which to emit light. As shown, a user is at 70% toward a goal of 100%. Therefore, activity display controller 1545 causes symbol 1554 (e.g., 10%), symbol 1553 (e.g., 70%), and intervening symbols to illuminate (i.e., shown as shaded). Note that activity display controller 1545 may illuminate symbol 1552 upon reaching a goal, and may further illuminate symbols 1557 to indicate a user's goal is surpassed (e.g., a user is at 110% of a goal).

FIG. 15D is a diagram depicting an example of a heart rate display controller interacting with a display portion, according to some examples. Diagram 1560 illustrates a display portion 1561 coupled to a heart rate display controller 1544. Heart rate display controller 1544 can determine that a heart rate is to be displayed, and can identify a quantity of lights and/or micro-perforations from which to emit light, the quantity of lights being proportional to a heart rate. As shown, heart rate display controller 1544 is configured to convey information related to heart rate, and thus causes symbol 1562 to illuminate (i.e., shown as shaded). Heart rate display controller 1544 is configured to determine a user's heart rate relative to a minimum heart rate (“Min HR”) associated with symbol 1566 and to a maximum heart rate (“Max HR”) associated with symbol 1564. Further, heart rate display controller 1544 is configured to determine an approximate value of the heart rate relative to gradations from, for example, from 62 beats per minute (“BPM”), which is associated with symbol 1565, to 150 BPM, which is associated with symbol 1567. Note that in some examples, each symbol illuminated from symbol 1565 indicates an additional 11 beats per minute (e.g., +/−2 to 4 bpm). In some embodiments, heart rate display controller 1544 can include a heart rate range adjuster 1548 that is configured to track a user's maximum and minimum heart rates during one or more activities and can adjust the maximum heart rate values and minimum heart rate values associated with symbols 1567 and 1566, respectively. Therefore, based on the wellness and health of a user's cardiovascular system and other factors, heart rate range adjuster 1548 can customize the gradations of symbols from symbol 1565 to symbol 1567 for a particular user. Note that the examples of the above-described display controllers are non-limiting examples can include controllers for displaying other information, such as a rate at which calories are burned, among other things.

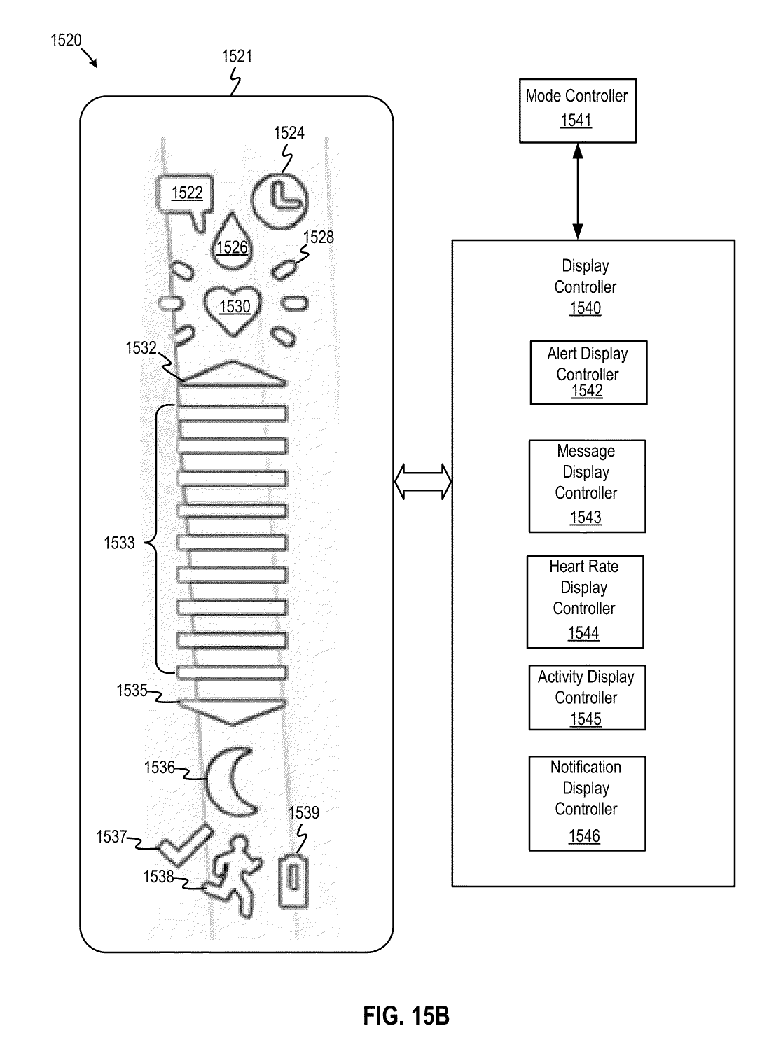

FIG. 16 is an example of a flow to form a wearable pod, according to some embodiments. At 602, a pod cover is received. For example, flow 600 can being by receiving a top pod cover including interface portions including one or more touch-sensitive portions and one or more display portions. In some examples, a top pod cover is configured to have a surface oriented away (e.g., away from a surface of a user) from a point of attachment to or positioning adjacent a user. At 604, one or more touch-sensitive surface portions may be coupled to logic for detecting contact upon the touch sensitive surface. At 606, a display portion is aligned adjacent to one or more sources of light such that perforations of the display portion are aligned to respective light sources. The one or more sources of light may be configured to emit light via a predominately opaque surface, at least in some examples. At 608, anchor portions or structures are formed at one or more distal ends of a touch-sensitive wearable pod. In some examples, a wearable pod and its top pod cover can be elongated in dimensions such that the wearable pod has two or more sides longer than the other two or more sides. In one case, the longer sides extend across a surface of an appendage (e.g., across a wrist) of a user. Shorter sides can be at the distal ends relative to the center or centroid of a wearable pod and/or its cradle. At 610, the top pod cover is isolated from logic and other portions of a touch-sensitive wearable pod. At 612, the wearable pod is sealed. For example, a top pod cover can be sealed and a bottom pod cover can be sealed to form a fluid-resistant (e.g., gas-resistant, liquid-resistant, etc.) barrier.

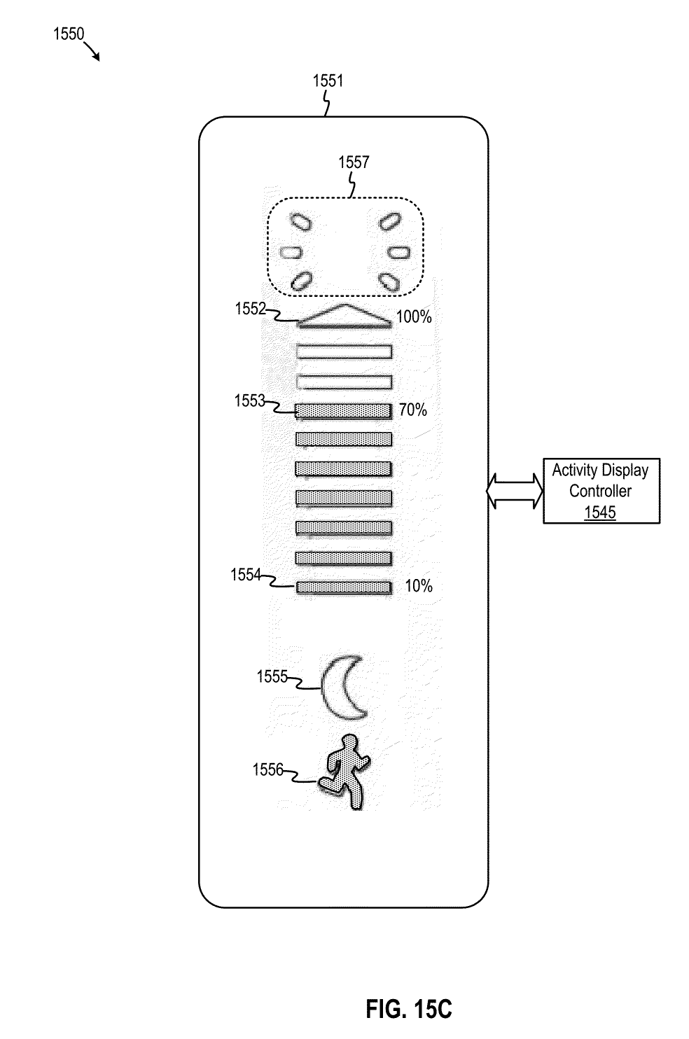

FIG. 17 illustrates an exemplary computing platform disposed in a wearable pod configured to facilitate a touch-sensitive interface in an opaque or predominately opaque surface in accordance with various embodiments. In some examples, computing platform 1700 may be used to implement computer programs, applications, methods, processes, algorithms, or other software to perform the above-described techniques.

In some cases, computing platform can be disposed in wearable device or implement, a mobile computing device, or any other device.

Computing platform 1700 includes a bus 1702 or other communication mechanism for communicating information, which interconnects subsystems and devices, such as processor 1704, system memory 1706 (e.g., RAM, etc.), storage device 17012 (e.g., ROM, etc.), a communication interface 1713 (e.g., an Ethernet or wireless controller, a Bluetooth controller and radio/transceiver, or other logic to communicate via a variety of protocols, such as IEEE 802.11a/b/g/n (WiFi), WiMax, ANT™, ZigBee®, Bluetooth®, Near Field Communications (“NFC”), etc.) to facilitate communications via a port on communication link 1721 to communicate, for example, with a computing device, including mobile computing and/or communication devices with processors.

One or more antennas may be implemented as a portion of communication interface 1713 to facilitate wireless communication. Also, one or more antennas may be formed external to a wearable pod (e.g., external to a cradle and/or one or more pod covers).

Processor 1704 can be implemented with one or more central processing units (“CPUs”), such as those manufactured by Intel® Corporation, or one or more virtual processors, as well as any combination of CPUs and virtual processors. Computing platform 1700 exchanges data representing inputs and outputs via input-and-output devices 1701, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 1700 performs specific operations by processor 1704 executing one or more sequences of one or more instructions stored in system memory 1706, and computing platform 1700 can be implemented in a client-server arrangement, peer-to-peer arrangement, or as any mobile computing device, including smart phones and the like. Such instructions or data may be read into system memory 1706 from another computer readable medium, such as storage device 1708. In some examples, hard-wired circuitry may be used in place of or in combination with software instructions for implementation. Instructions may be embedded in software or firmware. The term “computer readable medium” refers to any tangible medium that participates in providing instructions to processor 1704 for execution. Such a medium may take many forms, including but not limited to, non-volatile media and volatile media. Non-volatile media includes, for example, optical or magnetic disks and the like. Volatile media includes dynamic memory, such as system memory 1706.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that constitute bus 1702 for transmitting a computer data signal.

In some examples, execution of the sequences of instructions may be performed by computing platform 1700. According to some examples, computing platform 1700 can be coupled by communication link 1721 (e.g., a wired network, such as LAN, PSTN, or any wireless communication link or network, such a Bluetooth LE or NFC) to any other processor to perform the sequence of instructions in coordination with (or asynchronous to) one another. Computing platform 1700 may transmit and receive messages, data, and instructions, including program code (e.g., application code) through communication link 1721 and communication interface 1713. Received program code may be executed by processor 1704 as it is received, and/or stored in memory 1706 or other non-volatile storage for later execution.

In the example shown, system memory 1706 can include various modules that include executable instructions to implement functionalities described herein. In the example shown, system memory 1706 includes a touch sensitive I/O control module 1770, a display controller module 1772, an activity determinator module 1774, and a physiological signal determinator module 1776, one or more of which can be configured to provide or consume outputs to implement one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

In some embodiments, a wearable pod or one or more of its components (e.g., a touch-sensitive I/O controller or a display controller), or any process or device described herein, can be in communication (e.g., wired or wirelessly) with a mobile device, such as a mobile phone or computing device, or can be disposed therein.

In some cases, a mobile device, or any networked computing device (not shown) in communication with a wearable pod (or a touch-sensitive I/O controller or a display controller) or one or more of its components (or any process or device described herein), can provide at least some of the structures and/or functions of any of the features described herein. As depicted in FIG. 11 and/or subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in any of the figure can represent one or more algorithms. Or, at least one of the elements can represent a portion of logic including a portion of hardware configured to provide constituent structures and/or functionalities.

For example, a wearable pod or one or more of its components (e.g., a touch-sensitive I/O controller or a display controller), any of its one or more components, or any process or device described herein, can be implemented in one or more computing devices (i.e., any mobile computing device, such as a wearable device, an audio device (such as headphones or a headset) or mobile phone, whether worn or carried) that include one or more processors configured to execute one or more algorithms in memory. Thus, at least some of the elements in FIG. 11 (or any other figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit.

For example, a wearable pod or one or more of its components (e.g., a touch-sensitive I/O controller or a display controller), including one or more components, or any process or device described herein, can be implemented in one or more computing devices that include one or more circuits. Thus, at least one of the elements in FIG. 11 (or any other figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

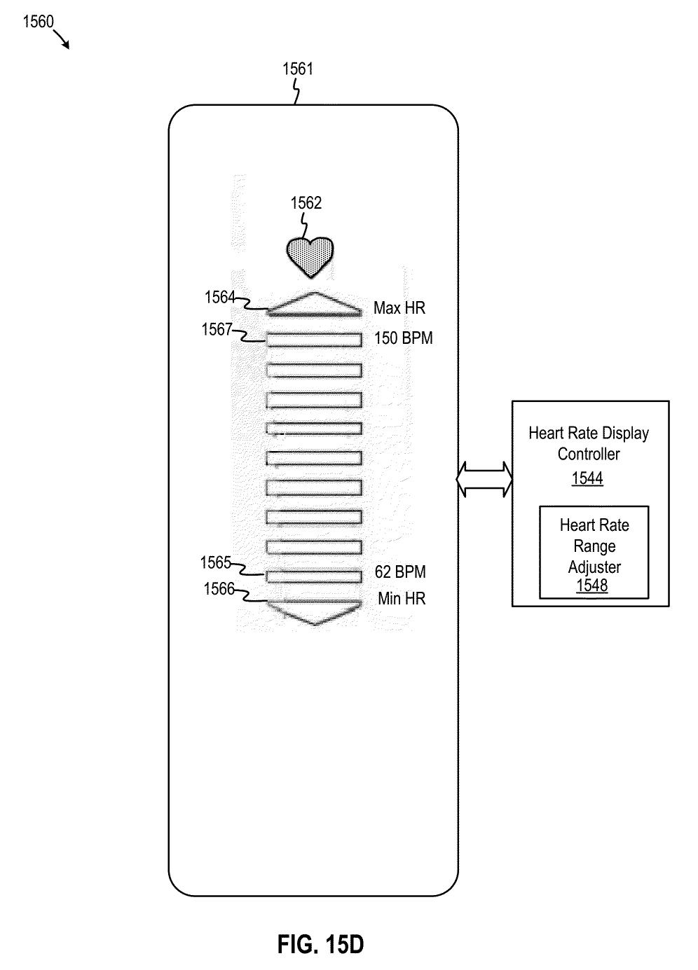

FIG. 18 is an exploded perspective view of an example of a wearable pod having, for example, a metal surface, according to some embodiments. Diagram 1800 includes a pod cover 1802 composed of conductive material, such as anodized aluminum in which the interior metal is conductive, a pod cover 1806 composed of similar material, and a cradle 1807 configured to be disposed within an interior region defined by pod covers 1802 and 1806. Cradle 1807 is further configured to house circuitry, including but not limited to a bioimpedance circuit, a galvanic skin response circuit, an RF transceiver (e.g., a Bluetooth Low Energy transceiver), and other electronic components and devices. As shown, cradle 1807 includes attachment portions 1877a and 1877b extending from distal ends of cradle 1807, attachment portions 1877a and 1877b being configured to adhere to an interface material that can constitute one or more anchor portions. Diagram 1800 also illustrates an isolation belt 1815 being formed at a region 1819 along or adjacent one or more longitudinal sides (e.g., sides 1817a and 1817b) of cradle 1807. Region 1819 along sides 1817a and 1817b can include one or more edges of pod cover 1802 disposed adjacent to one or more edges of pod cover 1806. A portion 1815a of isolation belt 1815 may be disposed between one or more edges of pod cover 1802 and one or more edges of pod cover 1806 to electrically isolate at least a portion of pod cover 1802 from pod cover 1806 and/or cradle 1807 or other circuitry that need not be related to detecting touch.

Further to FIG. 18, light sources 1841, such as light-emitting diodes (“LEDs”) or other sources of light, can be positioned to emit light to respective symbols in display portion 1804. Also shown is a mounting frame 1803 in which to house light sources 1841 in corresponding apertures 1883. Mounting frame 1803 also includes another aperture 1882 to enable a conductive path 1880 to extend from pod cover 1804 to a touch-sensitive I/O controller circuit (not shown). Other examples of light sources 1841 include, but are not limited to, interferometric modulator display (IMOD), electrophoretic ink (E Ink), organic light-emitting diode (OLED), or other display technologies.

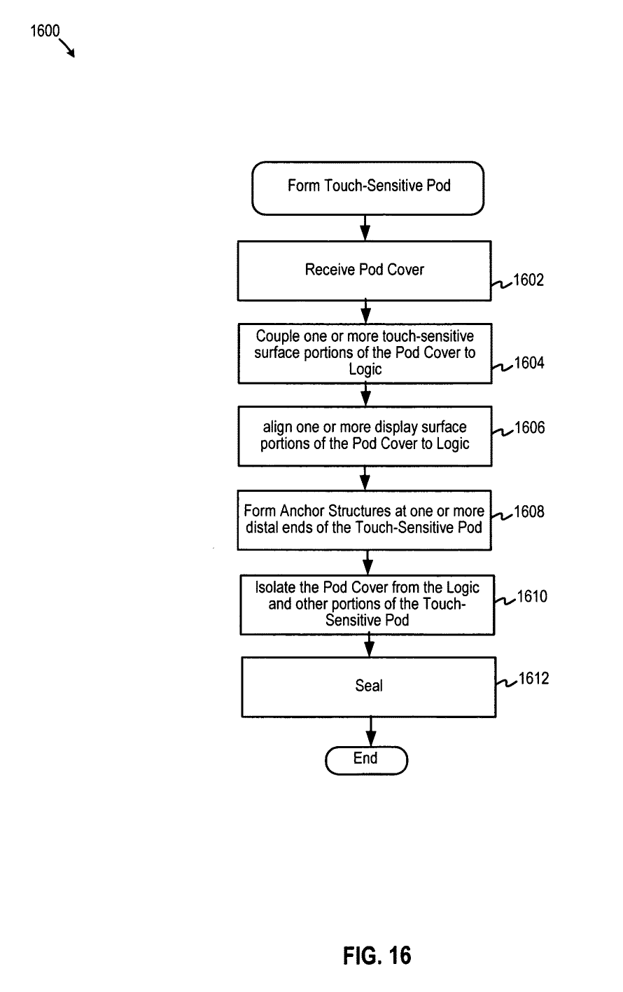

FIG. 19 is an exploded front view of an example of a wearable pod having, for example, a metal surface, according to some embodiments. Diagram 1900 illustrates elements having structures and/or functions as similarly-named or similarly-numbered elements of FIG. 18. Note that edges 1903 of pod cover 1802 and edges 1906 of pod cover 1806 are configured to be adjacent each other, when assembled, at or near region 1919. According to some embodiments, a portion 1915a (e.g., a ridge or rib) is configured to isolate edges 1903 and edges 1906 from contacting each other, thereby facilitating touch-sense of capabilities of pod cover 1802 (e.g., by preventing electrical shorts or other conditions or phenomena).

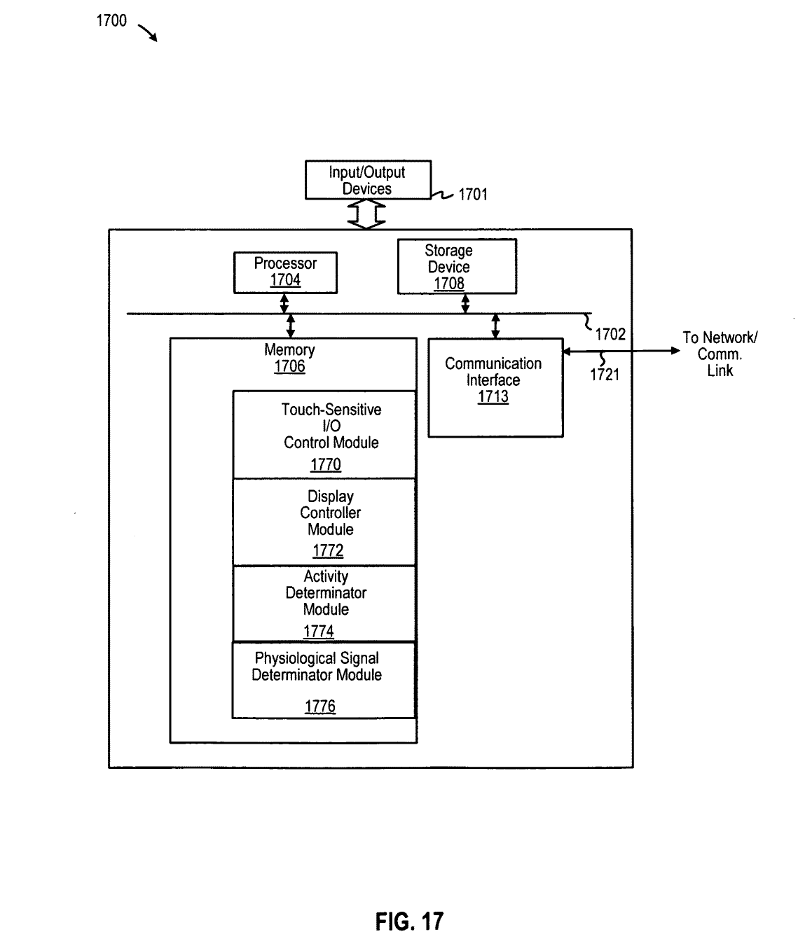

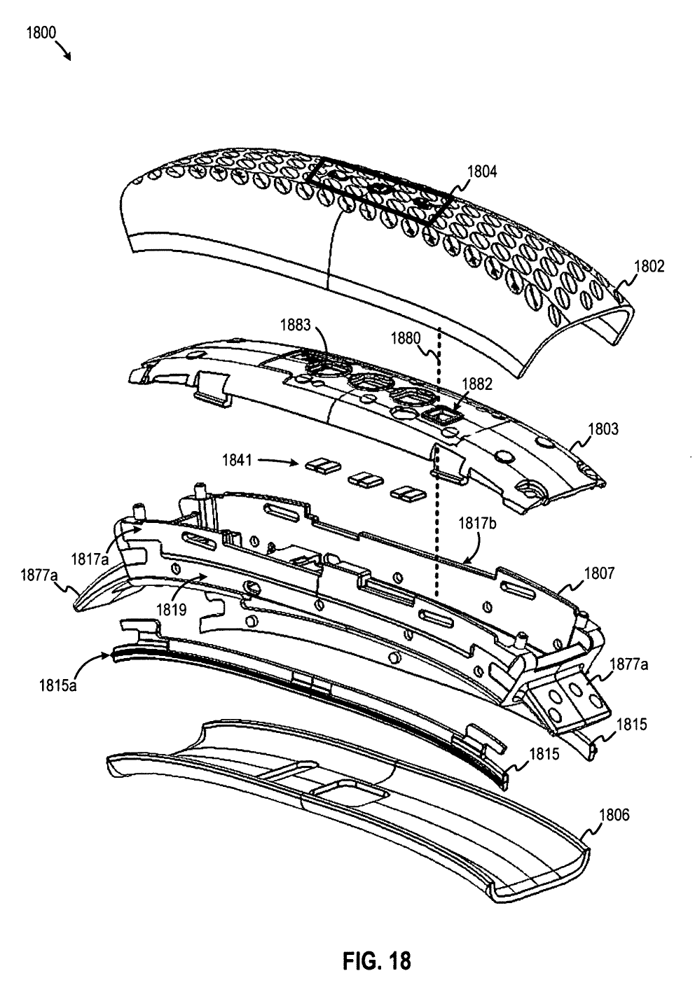

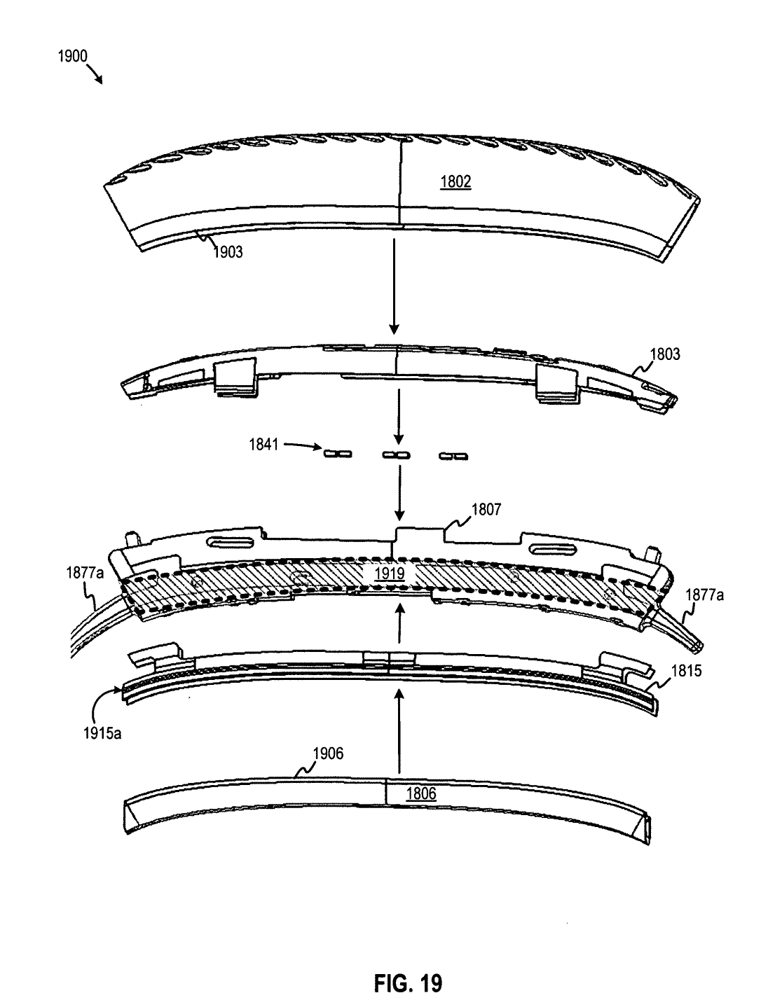

FIGS. 20A to 20B are respective exploded perspective and exploded front views of a wearable pod including anchor portions, according to some embodiments. Diagram 2000 illustrates elements having structures and/or functions as similarly-named or similarly-numbered elements of FIGS. 18 and 19. Further, diagram 2000 illustrates formation of anchor portions 1809a and 1809b on attachment portions at the distal ends of cradle 1807. Also shown is portion 1915a of an isolator belt that can be formed during the formation of anchor portions 1809a and 1809b. As such, the isolator belt and ridge 1915a can be composed of a material used to form portions 1809a and 1809b. Diagram 2050 illustrates elements having structures and/or functions as similarly-named or similarly-numbered elements of FIGS. 18 to 20A. Further, diagram 2050 illustrates formation of anchor portions 1809a and 1809b formed, for example, contemporaneous with the formation of portion 1915a of an isolation belt and the formation of an under-layer material 2017, all of which can be composed of a common material (e.g., an interface material). In some embodiments, anchor portions 1809a and 1809b, portion 1915a of an isolation belt, and under-layer material 2017 can be composed of a thermoplastic. For example, the thermoplastic can include polycarbonate or other similar materials.

FIG. 20C is a bottom perspective view of a pod cover implementing a sealant during assembly, according to some embodiments. Diagram 2070 illustrates a pod cover 2002 having edges 2013 at least two of which may be disposed adjacent to edges of a bottom pod cover once assembled. Diagram 2070 also shows a sealant 2078 applied on an inner surface portion of pod cover 2002 at or adjacent to one or more edges 2013 of pod cover 2002 to form a fluid-resistant bond to a cradle, an isolation belt, or another structure. In one example, a fluid-resistant bond or barrier is formed to withstand intrusions of water at 1 ATM. Arrangements of micro-perforations 2082 are shown to extend from an inner surface 2079 of a portion of pod cover 2002 to an outer surface 2081 of pod cover 2002.

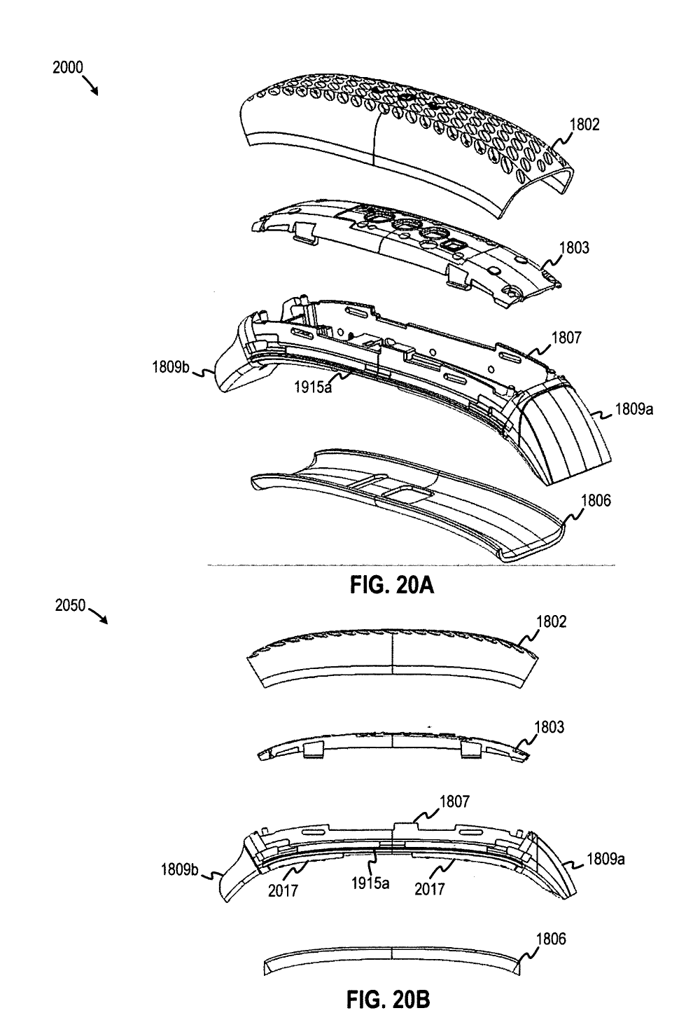

FIG. 20D is a diagram depicting a perspective front view of a wearable pod being assembled as part of a wearable device, according to some embodiments. Diagram 2080 illustrates a pod cover 2002 and a pod cover 2006 being brought together to form respective seals to encapsulate the interior structures and circuitry. For example, when assembled, pod covers 2002 and 2006 enclose a light diffuser 2099 (e.g., for diffusing LED-generated light), which may be optional, mounting frame 2003, and cradle 2007. Further, straps 2020 and 2022 are respectively molded on anchor portions 1809b and 1809a, respectively, whereby anchor portions 1809a and 1809b are composed of interface materials configured to securely couple cradle 2007 to straps 2020 and 2022. In some embodiments, cradle 2007 comprises a metal material and straps 2020 and 2022 may be composed of a pliable material, such as an elastomer. Note that logic may be disposed within cradle 2007 under mounting frame 2003. Examples of such logic include a bioimpedance circuit disposed in cradle 2007 and configured to couple to a first subset of conductors to receive electrical signals embodying physiological data originating from points in space adjacent to blood vessels in tissue. Also, such logic can include a galvanic skin response circuit disposed in cradle 2007 and configured to couple to a second subset of conductors to receive electrical signals indicative of a conductance value across a portion of tissue. Further, a cross-section view X-X′ of a portion of an isolation belt and the edges of pod covers 2002 and 2006 are depicted in FIGS. 21A and 21B.

FIGS. 21A and 21B are diagrams depicting a cross-section of a portion of an isolation belt, according to some examples. Diagram 2100 is a cross-section view of an assembled wearable pod including a pod cover 2102 attached to interior structures and a pod cover 2106 that is also attached to interior structures. Diagram 2100 also illustrates an inset 2130 diagram that includes a cross-section view of an isolation belt. As shown in inset 2130 diagram of FIG. 21B, an isolation belt 2115 formed on or adjacent a cradle 2107. Isolation belt 2115 includes a portion 2115a (or ridge 2115a) that isolates pod cover 2102 from pod cover 2106. According to some embodiments, a sealant 2170 is configured to form a fluid-resistant bond between pod cover 2102 and isolation belt 2115 and/or 2107.

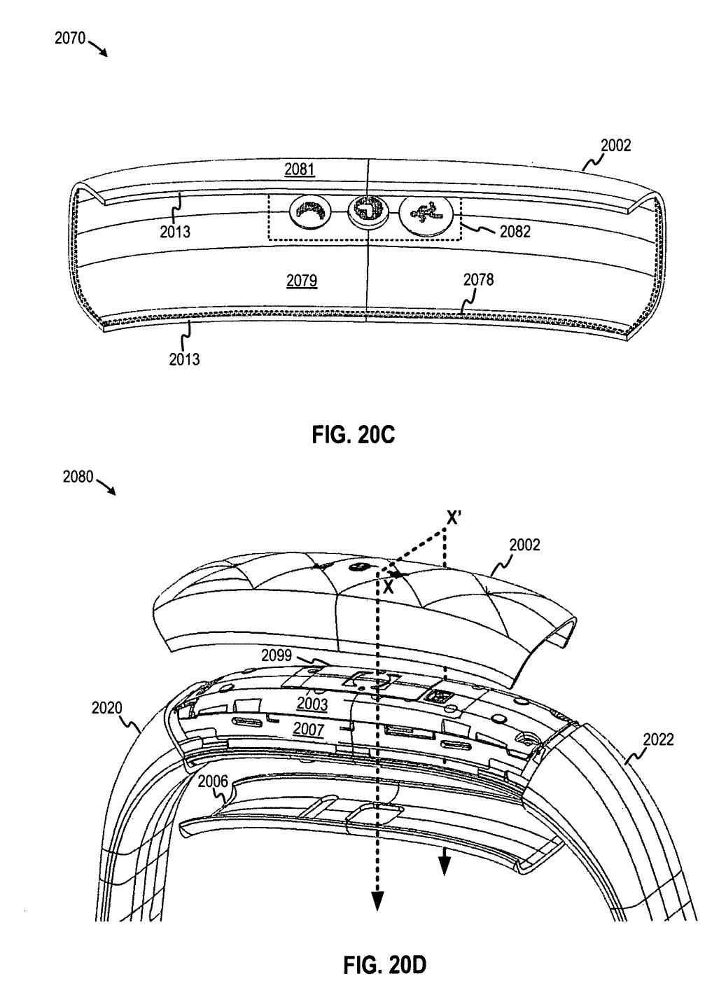

FIG. 22 illustrates an example of a flow to form a touch-sensitive pod cover for a wearable pod, according to some examples. Flow 2200 includes forming a pattern at 2202 on a substrate, such as a metal substrate. At 2202, a cosmetic pattern may be formed on a top surface using stamping or CNC-based machine patterning. Prior to 2202, a pod cover can be singulated or separated from other metal. In some examples, the pod cover is an aluminum metal substrate. At 2204, the contours (e.g., the dimensions and spatial characteristics) of the pod cover are formed. Forming the contours include forming shapes of the sides and top surfaces. At 2206, a coating can be formed on the surface of the pod cover. For example, an aluminum pod cover can be anodized to form covered surface on the pod cover. At 2208, a portion of the pod cover is etched to provide access to the aluminum metal substrate (e.g., under the coating) for purposes of electrically coupling the pod cover to, for example, a touch-sensitive I/O control circuit to detect a touch event. For example, a portion of an inner surface of a top pod cover may be etched to facilitate formation of an electrical path to couple one or more touch-sensitive portions of the pod cover to touch-detection logic. At 2210, perforations may be formed in a touch-sensitive portion of the pod cover. In some examples, the perforations and/or micro-perforations can be formed by drilling a number of perforations with a laser to form one or more symbols. At 2212, an optically-transparent sealant can be applied to the perforations and/or micro-perforations for form a display portion.

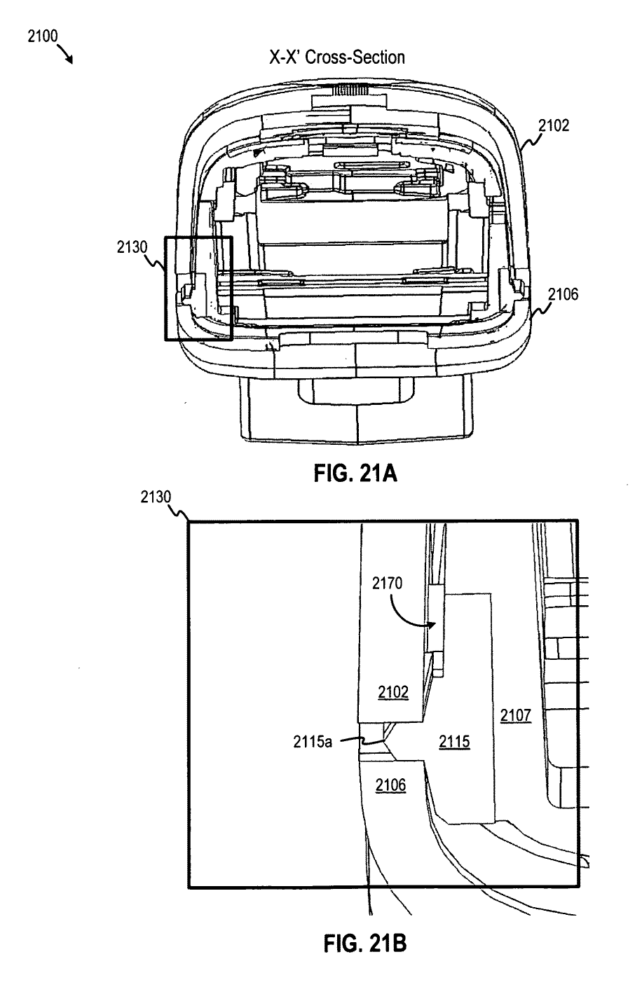

FIG. 23 illustrates an example of a flow for a touch-sensitive wearable pod, according to some embodiments. Flow 2300 includes setting a cradle and components in a first mold. For example, the components can include a temperature sensor and pins (e.g., pogo pins) to form a USB connector (or other types of connectors). At 2304, an insulator belt is formed and, at 2306, one or more anchor portions may be formed at one or more attachment portions at one or more distal ends of a cradle. In some examples, the formation of anchor portions includes molding over metal surfaces of the one or more attachment portions with an interface material having properties to facilitate bonding to an elastomer. In at least one example, a thermoplastic material is molded over a magnesium metal surface of one or more cradle attachment portions. In various embodiments, the various thermal plastic materials are suitable for the above-described implementation. In at least one embodiment, the thermal plastic material includes polycarbonate or equivalent. At 2308, a portion of a pod cover can be etched to provide for electrical contact to a touch-detection circuit. At 2310, one or more pod covers are selected and a sealant 2312 may be applied thereto. For example, an epoxy may be applied adjacent to one or more edges of a top pod cover, whereby the epoxy may contact a one or more surface of a cradle disposed within an interior region formed between the top pod cover and a bottom pod cover. Note that flow 2300 is not intended to be exhaustive in may be modified within the scope of the present disclosure.

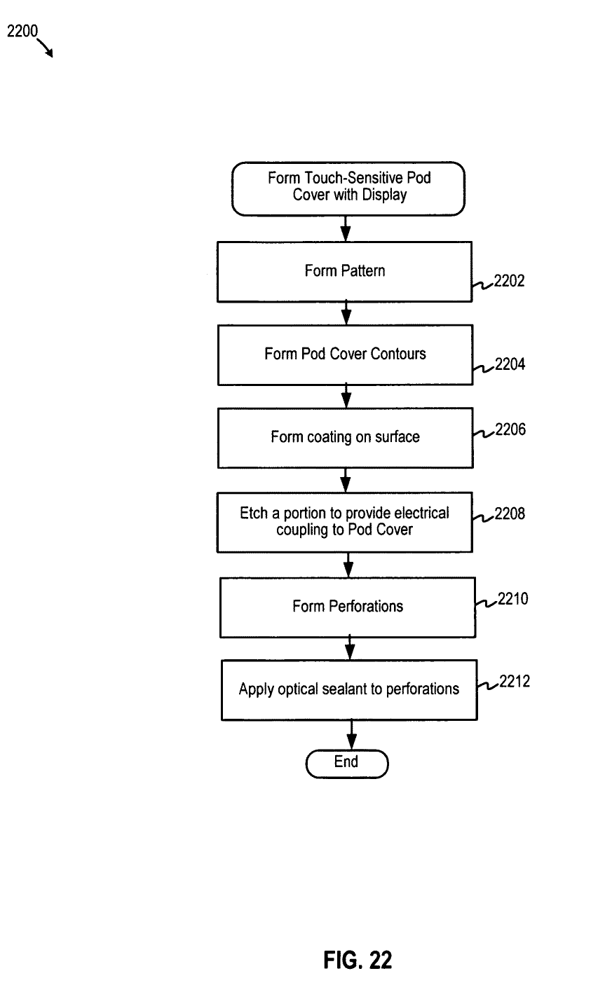

FIG. 24 is a diagram depicting an antenna configured for implementation in a wearable pod having a metallized interface, according to some embodiments. Diagram 2400 includes an antenna 2402 having terminals 2403 and 2405 formed in a first end, the terminals being configured to couple a transceiver disposed in a region enclosed by or defined by a top pod cover and a bottom pod cover, neither of which are shown. As such, antenna 2402 is configured to be implemented external to a metal-based enclosure formed by the pod covers of a wearable pod. Diagram 2400 further shows that antenna 2402 includes a stacked portion 2406 and an extended portion 2408. Stacked portion 2406 is a portion of metal (e.g., planar metal) that is configured to be oriented in a “stacked” position over an attachment portion, whereas extended portion 2408 is a portion of metal that is configured to “extend” beyond the attachment portion. In some embodiments, extended portion 2408 includes a greater amount of surface area than stacked portion 2406. Further, diagram 2400 illustrates a gap 2413 in antenna 2402 that separates a metal portion 2410 from a metal portion 2420, the gap 2413 extending from adjacent one corner 2490 to an opposite corner 2492. Opposite corner 2042 is disposed diagonally from the other corner 2490 as shown. Note that metal portion 2410 is coupled to metal portion 2420 at a transition portion 2419, which, at least in some examples, has the smallest width dimension across the surface area of antenna 2402. In some examples, metal portion 2410 and metal portion 2420 may have equivalent surface areas. In at least one example, metal portion 2410 is disposed predominantly in stacked portion 2406, whereas metal portion 2420 is disposed predominantly in extended portion 2408. In some embodiments, stacked portion 2406 is defined, at least in one example, by a portion 2411 of a non-conductive gap 2413. Diagram 2400 also illustrates a number of holes 2418 in antenna 2402 that are configured to align with alignment posts (not shown) on an under-anchor portion during antenna placement. According to some embodiments, antenna 2402 can be configured as a Bluetooth® antenna, such as Bluetooth low energy (Bluetooth LE) antenna, the specifications of which are maintained by Bluetooth Special Interest Group (“SIG”) of Kirkland, Wash., USA. According to other embodiments, antenna 2402 can be designed to receive radio frequency (“RF”) signals associated with other wireless communication protocols, including, but not limited to various WiFi protocols, cellular data signals, etc. According to various other embodiments, other antenna shapes for antenna 2402 are also the scope of the present disclosure. As such, antenna 2402 can serve as antenna for multiple types of RF signals, such as Bluetooth and WiFi.

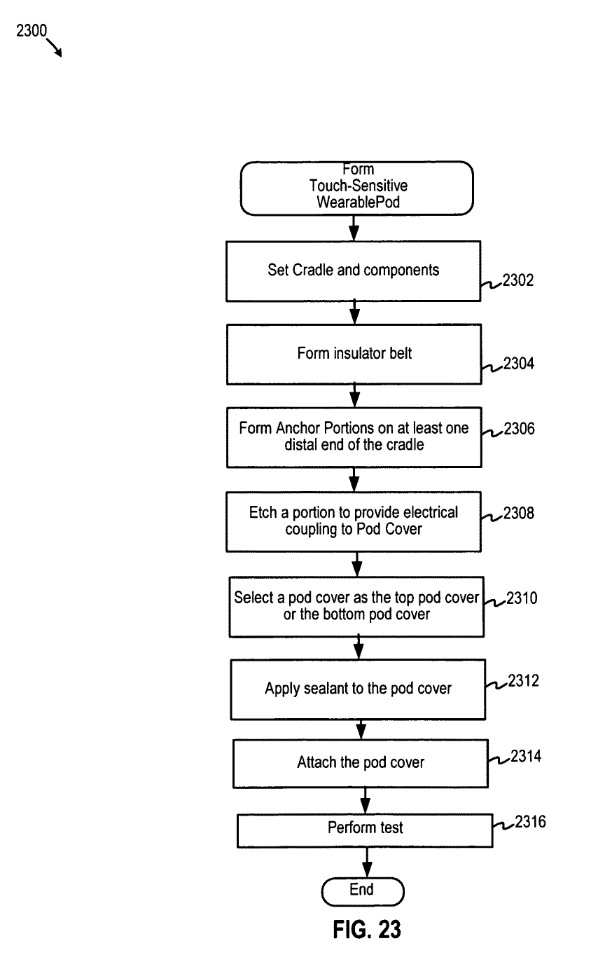

FIGS. 25A to 25C depict examples of an antenna oriented relative to an attachment portion of a cradle, according to some embodiments. FIG. 25A is a diagram 2500 depicting a front view of a cradle 2507 having an attachment portion 2577a extending from a distal end of cradle 2507, and an attachment portion 2577b extending from another distal end of cradle 2507. In this example, cradle 2507 has elongated dimensions, whereby attachment portion 2577a extends longitudinally (longitudinal direction 2501) and/or circumferentially away from a center point 2503 of cradle 2507. In one example, cradle 2507 is composed of metal, such as magnesium, and is configured to be disposed between a top pod cover the bottom pod cover (not shown). Cradle 2507 was further configured to have an interior region for housing circuitry and to accept conductors, such as terminals 2403 and 2405 of FIG. 24, that extend externally from cradle 2507.

Further to diagram 2500, a stacked portion 2406 of planar metal disposed at a first distance to a portion of attachment portion 2577a of metal cradle 2507, whereas a portion of extended portion 2408 may be disposed at a second distance (from the portion of attachment portion 2577a), which is greater than the first distance. In some non-limiting examples, a portion of stacked portion 2406 may parallel or substantially parallel (e.g., non-intersecting in a region) to a portion of attachment portion 2577a. In some cases, a portion of stacked portion 2406 may be shaped to have one or more radii of curvature as a portion of attachment portion 2577a.

In some examples, antenna 2402 can include a stacked portion 2406 that traverses a first region from a radial plane 2513 to a radial plane 2515, the first region including attachment portion 2577a. Extended portion 2408 is shown to traverse a second region at an angular distance, d2, which is greater than an angular distance between radial plane 2513 and radial plane 2515. Note that the second region excludes attachment portion 2577a, wherein radial plane 2513 and radial plane 2515 extend radially from a line 2512 parallel to a bottom plane 2588 coextensive with a portion of a bottom of cradle 2507. Radial plane 2517 extends from line 2512 without passing through attachment portion 2577a.

According to other examples, attachment portion 2577a and a short-range communication antenna 2402 may include bottom surface portions that are coextensive with a curved surface 2511 having one or more radii centered at a point (e.g., on line 2512) in a region below the bottom pod cover. In various implementations, curved surface 2511 may be configured to facilitate attachment to a strap configured to encircle a portion of a wrist (or other circularly-shaped appendages).

Attachment portion 2577b is configured to extend at a greater distance from a side of a cradle 2507 than attachment portion 2577a to, for example, accommodate different structures and/or functions. As shown, attachment portion 2577b has a surface coextensive with a curved surface 2599 extending from a radial plane 2505 to a radial plane 2598. Radial planes 2505 and 2598 can extend radially from line 2510. According to some embodiments, attachment portion 2577b can be configured to support circuitry, such as conductors, electrodes, a collection of electrodes, electrode bus, and circuitry, such as near-field communications devices (e.g., NFC semiconductor chip).

FIG. 25B is a diagram 2550 depicting a magnified front view of a cradle 2507 having an attachment portion 2577a extending from a distal end of cradle 2507. As shown, extended portion 2408 is shown to traverse a region 2559 at an angular distance, d2, which is greater than angular distance, d1. Note that stacked portion 2406 that traverses a region 2558 that includes attachment portion 2577a. Note that region 2558 can include in interface material, such as polycarbonate, when forming an anchor portion. Similarly, region 2559 may include some interface material as well.

FIG. 25C is a diagram 2570 depicting a magnified perspective view of a cradle 2507 having an attachment portion 2577a extending from a distal end 2599 of cradle 2507. In the example shown, stacked portion 2406 and extended portion 2408, at least in one example, are separated by a portion 2411 of a non-conductive gap 2413. Portion 2411 of non-conductive gap 2413 can include a portion of the plane 2580 that may be orthogonal or substantially orthogonal to plane 2582, which can be coextensive with a surface of attachment portion 2577a. Further, a portion of the interface material may be disposed in gap 2411 when an anchor portion is formed. According to other embodiments, a shortest distance between plane 2582 and stacked portion 2410 may be greater than the shortest distance(s) between extended portion 2408 and plane 2582 as the shortest distances between plane 2582 and stacked portion 2410 are configured to minimize interference for metallic surface of attachment portion 2577a during operation of antenna 2402.

FIG. 26 is an exploded perspective view of an anchor portion, according to some embodiments. Diagram 2600 includes a cradle 2607 having an under-anchor portion 2679a formed (e.g., molded) thereupon. An antenna 2402 is aligned such that posts 2610 pass through holes 2612 during assembly. According to some embodiments, antenna 2402 is secured to the surface of under-anchor portion 2679a by heat staking posts 2610 (e.g., deforming the tops of posts 2610 to expand at diameters larger than holes 2612). In one case, the material of posts 2610 are heated and pressure is applied thereto to deform the posts. An over-anchor portion 2679b can be formed (e.g., molded) over antenna 2402 and under-anchor portion 2679a to form a portion 2609a.

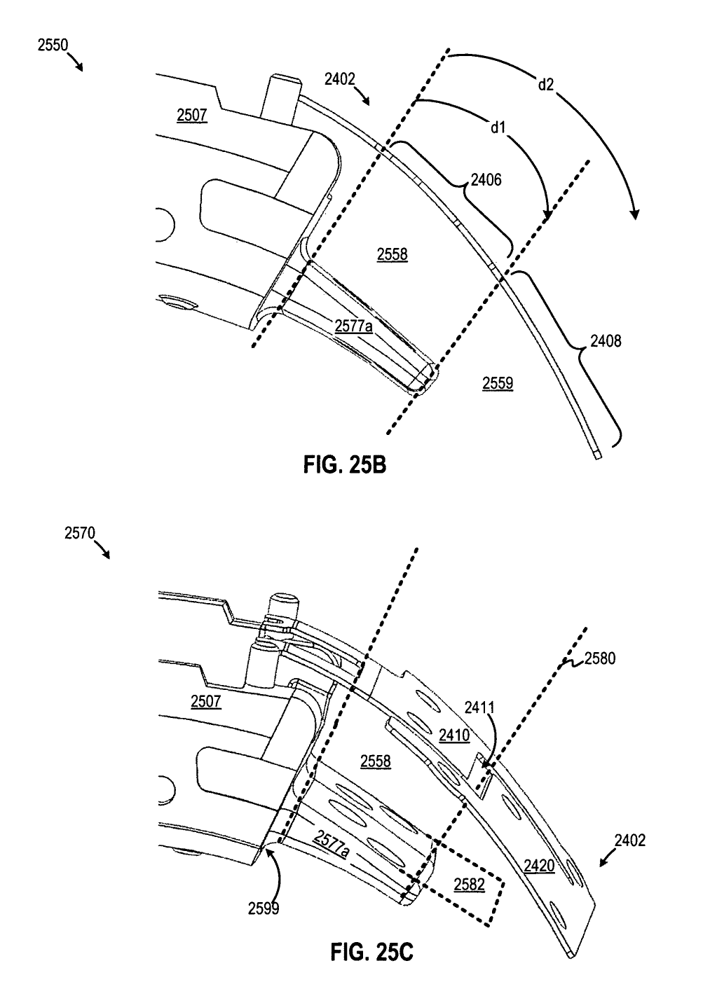

FIG. 27 is an example of a flow to manufacture a communications antenna in a wearable pod and/or device, according to some embodiments. In flow 2700, an antenna is selected, whereby the antenna has a first surface area that extends beyond a second surface area associated with an attachment portion a cradle for a wearable pod, the first surface area being greater than the second surface area. At 2074, an under-anchor portion on the attachment portion maybe formed. Forming the under-anchor portion can include configuring the surface of the under-anchor portion to receive the antenna at 2706. For example, the surface of the under-anchor portion can be configured to include posts extending from the surface of the under-anchor portion. In some cases, a portion of the interface material can be disposed in a first portion of a gap in the antenna, the gap being coextensive with a first plane that is orthogonal or is substantially orthogonal (i.e., more orthogonal than not, or +/−30% from a vector normal to the surface) to a second plane coextensive with a surface of the attachment portion. The under-anchor portion can be formed by shaping surface of the under-anchor portion to be coextensive with a curved surface having one or more radii centered at a point in a region below a bottom of the cradle.

Further, an antenna can be disposed at 2708 upon the surface of the under-anchor portion. For example, the holes in the antenna may be aligned with the posts, and the antenna can be placed on the surface of the under-anchor portion. For example, the antenna may be disposed on a surface of the under-anchor portion at a distance from a surface area associated with the attachment portion. In at least one example, the posts can be deformed to lock the antenna in position. At 2710, an over-anchor portion may be formed over the antenna and the under-anchor portion to form an anchor portion configured to attach to, for example, a strap composed of the elastomer. Further, the under-anchor and/or over-anchor portions may be composed of an interface material configured to bind to the cradle and to an elastomer. An example of an interface material is polycarbonate, and an example of an elastomer is a thermoplastic elastomer (“TPE”). In one embodiment, an elastomer includes a thermoplastic polyurethane (“TPU”) material.

In one embodiment, selecting the antenna can include selecting a short-range antenna including terminals coupled to a Bluetooth circuit in a cradle of a wearable pod. The antenna includes a stacked portion of planar metal configured to be disposed at a first distance from the attachment portion of metal cradle, and an extended portion of the planar metal configured to be disposed at a second distance, which is greater than the first distance. Also, selecting the antenna can include selecting a Bluetooth antenna to transmit and receive radio signals implementing a Bluetooth protocol. In addition, selecting the antenna can include selecting an antenna having a first metal portion electrically isolated from a second metal portion by a gap extending diagonally or substantially diagonal (i.e., more diagonal than not, or +/−30% from a line passing through two corners) from adjacent one corner of the antenna to an opposite corner of the antenna.

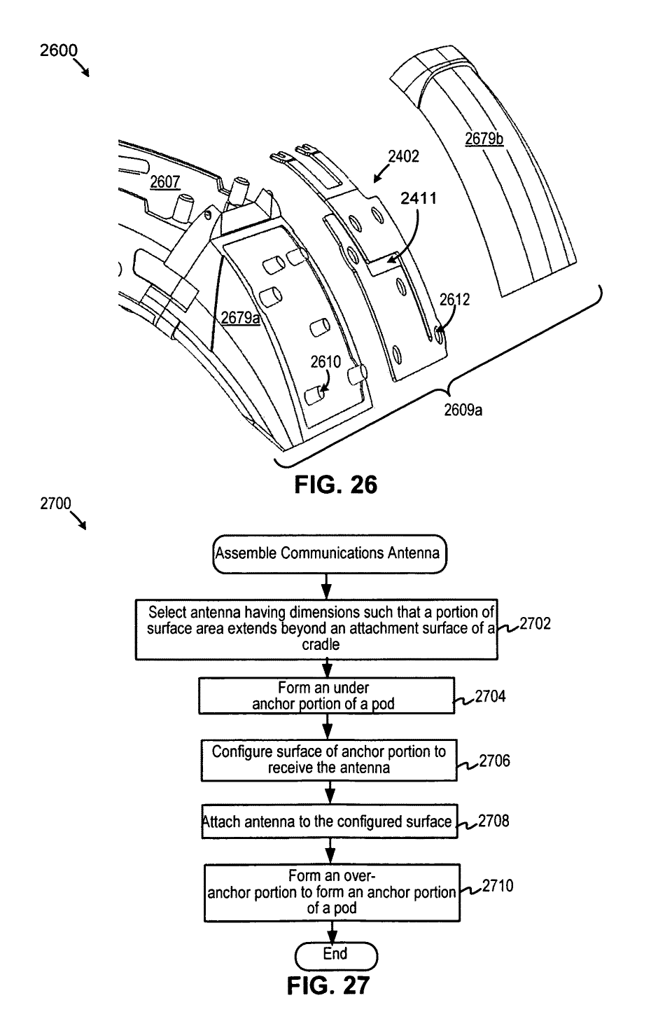

FIG. 28 is a diagram depicting an antenna configured for implementation in a wearable pod having a metallized interface, according to some embodiments. Diagram 2800 includes a cradle 2807 including an anchor portion 2809b at which a near field communication (“NFC”) system is disposed. Anchor portion 2809b is formed with a channel 2819 having a channel support floor 2820 and channel walls 2813. Channel 2819 is configured to support one or more layers of material above plane 2884, which is coextensive at least a portion of channel floor 2820. As shown, near field communication system 2870 includes a communication device 2880 and an antenna 2882, whereby near-field communication antenna 2882 has a first end disposed in channel 2819 of anchor portion 2809b. In this example, near field communication system 2870 is disposed external to cradle 2807. Further, near field communication system 2870 may be disposed external to a periphery of a first pod cover and a second pod cover (neither are shown) over cradle 2807. Communications device 2880 may have a potting compound formed thereupon.

In diagram 2800, antenna 2882 may include a subset of terminals (not shown) disposed at a first end of the antenna in channel 2819, the subset of terminals being coupled to near-field communication device 2880 mounted on the first end of antenna 2882. According to some embodiments, near-field communication device 2880 may include an active near-field communication device that may be configured to receive power from adjacent the near-field communication antenna upon which radio frequency radiation is received. This amount of power may be sufficient to cause near field communication device 2880 to transmit data including, for example, a communication device ID. Antenna 2882 includes a metal-based pattern configured to receive near-field communications signals and may include polyamide. According to some embodiments, a region between antenna 2882 and plane 2884 may include one or more other layers, one of which may include an electrode bus as described herein. As such, an electrode bus can provide support for antenna 2882 as well as near field communication device 2880.

Further to diagram 2800, a communications device identifier extractor 2890 is configured to program an identifier into a memory (not shown) in cradle 2807. The identifier uniquely identifies near field communications device 2880. As shown, communication device identifier extractor 2890 may be configured to transmit radiation 2898 to cause near field communications device 2880 to transmit an identifier as data 2896. Then, a communication device identifier extractor can program identifier as data 2894 into memory. In some cases, communication device identifier extractor 2890 may be used during assembly, final test and/or packaging stages of manufacture. A memory in cradle 2807 can store data representing the identifier of near-field communication device 2880, memory being disposed in a wearable pod. The identifier is accessible to facilitate activation of the near-field communication device. For example, consumer can couple the memory in Internet network to activate, for example, a credit card account.

According to some embodiments, near-field communication antenna is configured to facilitate radio reception and/or transmission of signals in accordance with near field communication interface and protocols, such as those set forth and/or maintain by International Organization for Standardization (ISO) and the International Electrotechnical Commission (IEC) of Geneva, Swtizerland.

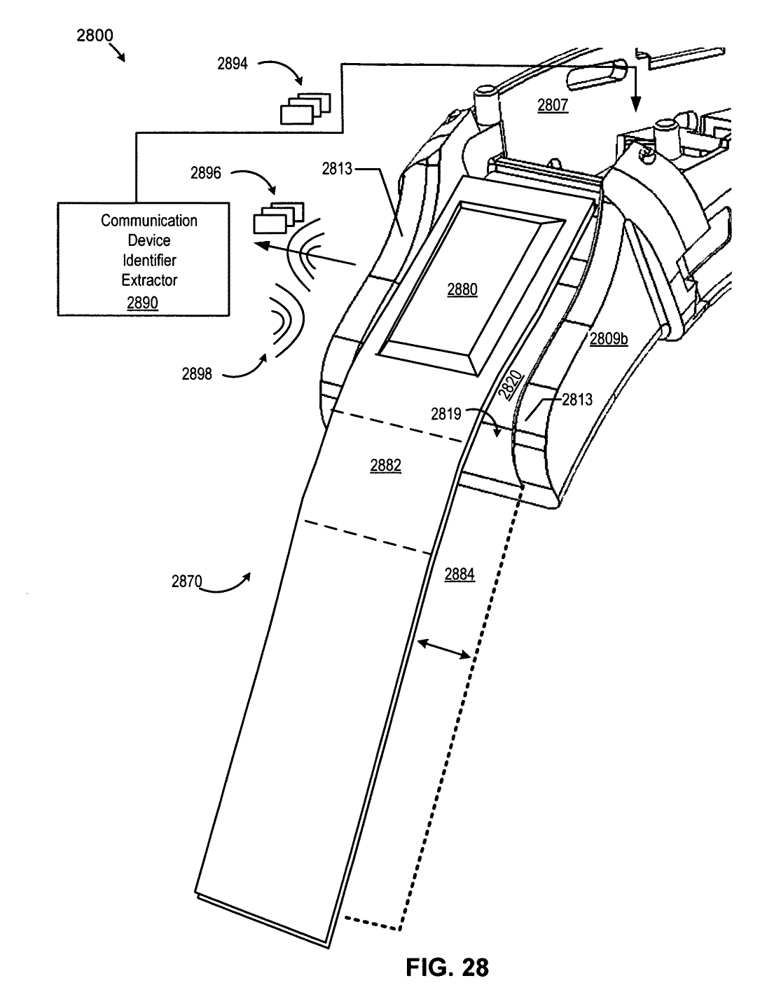

FIGS. 29A and 29B are perspective views of an attachment portion and an anchor portion, respectively, according to some embodiments. Diagram 2900 of FIG. 29A illustrates attachment portion 2977b prior to formation of an anchor portion 2809b, as shown in diagram 2950 in FIG. 29B.

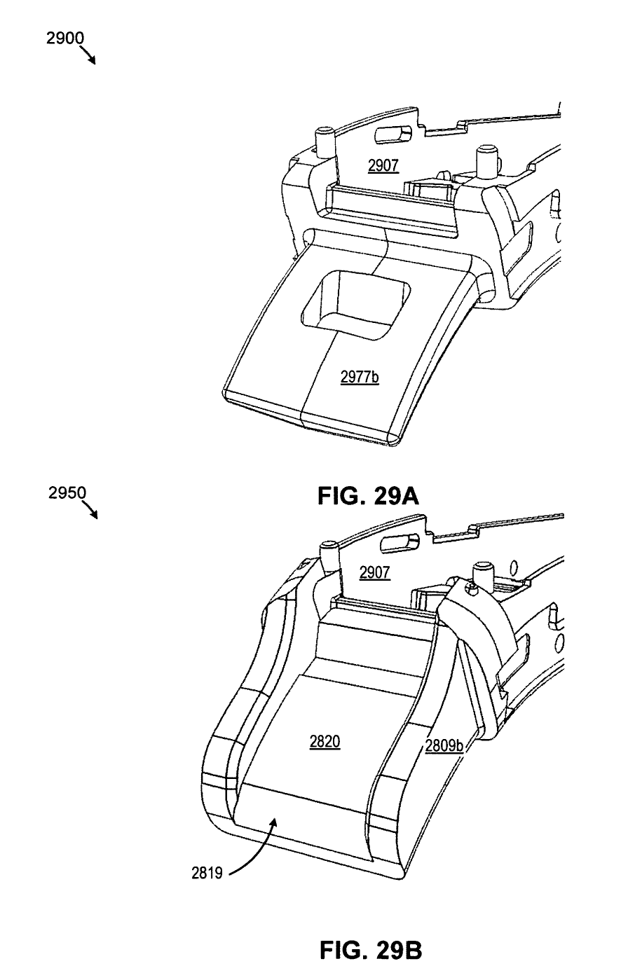

FIG. 30 is a diagram depicting another example of a near field communication antenna implemented in a wearable device, according to some examples. Diagram 3000 illustrates a near field communication antenna 3082 having terminals 3003 and 3005 being configured to couple via anchor portion 3009b to circuitry in a cradle 3007 (e.g., a metal cradle), the antenna including planar metal disposed in a layer of material, such as polyamide. A near-field communication device (not shown) in cradle 3007 can be coupled to the near-field communication antenna 3082 via terminal 3003 and 3005. In some examples, near-field communication antenna may include another set of terminals (not shown) to perform either transmit or receive operations, or both, of the near-field communication device (and/or to provide power to the antenna for communication or processing).

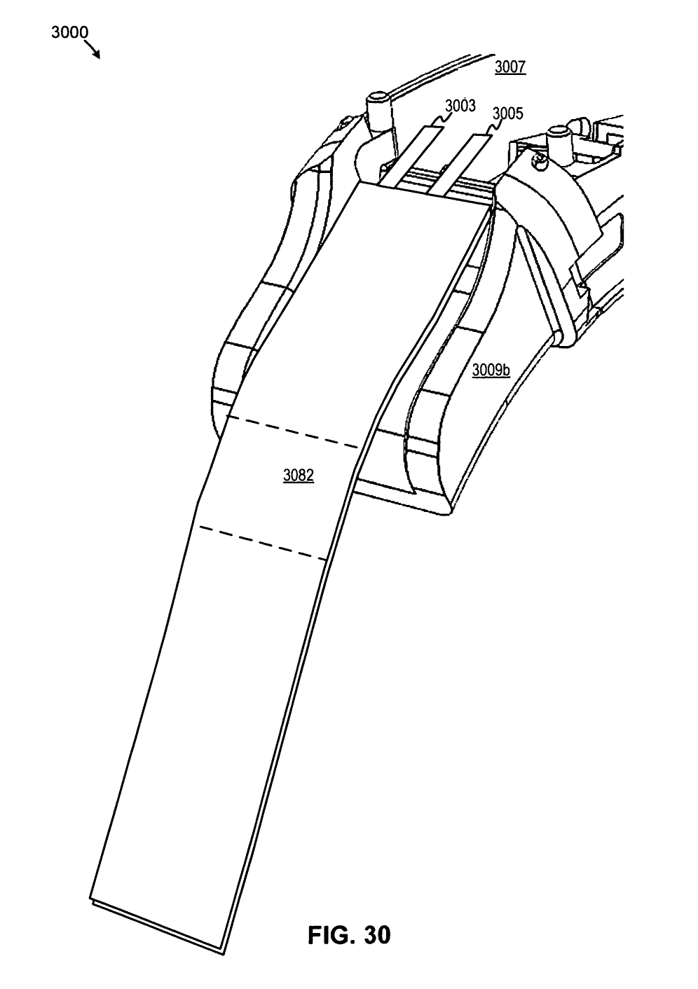

FIG. 31 is an example of a flow to manufacture a short-range communications antenna in a wearable pod and/or device, according to some embodiments. In flow 3100, an antenna is selected at 3102, whereby the antenna has a width dimension configured to be disposed in a wearable strap. For example the width dimension of the antenna is less than the width of the strap and/or wearable pod (e.g., a width less than a top or bottom pod cover). In another example, the width of the antenna is less than the distance between channel walls formed in an anchor portion. In particular, an antenna having a width dimension sized less than a width dimension of a channel may be selected. At 3104, a cradle having an attachment portion for a wearable pod can be selected, and an anchor portion may be formed on the attachment portion. The anchor portion can be composed of an interface material configured to bind to the cradle and to an elastomer, and the anchor portion can also include a channel to provide support. In one case, the anchor portion as a surface shaped to be coextensive with, for example, a curved surface having one or more radii centered at a point in a region below a bottom of the cradle.

At 3106, an inner portion of a wearable strap is formed coupled to an anchor portion including the channel. At 3108, a portion of the antenna may be disposed in the channel and/or a part of an inner portion of a wearable strap located adjacent a wearable pod. According to some embodiments, a portion of the antenna disposed in the channel may also include and/or be coupled to a near field communications device (e.g., a near-field communication semiconductor device). In particular, terminals of antenna can be coupled to circuitry of a near-field communication semiconductor device disposed on the antenna or substrate that includes an antenna.

At 3110, a determination is made whether near field communication logic is external. In particular, a determination is made whether the near field communication device is located external or internal to a cradle. If the near field communication device disposed within a cradle, flow 3100 moves to 3112 at which antenna conductors or terminals are attached coupled to internal logic, including a near-field communication device. Otherwise, flow 3100 moves to 3114 at which a near field communication device mounted on the antenna is encapsulated as an outer portion of the strap is formed at 3116. At 3118, identifier associated with logic in the near field communication device is identified. For example, an electromagnetic field can be applied adjacent to the antenna, and the identifier can be read. The identifier may be stored in memory at 3120. For example, identifier can be programmed in a memory residing in the cradle for subsequent activation by a user.

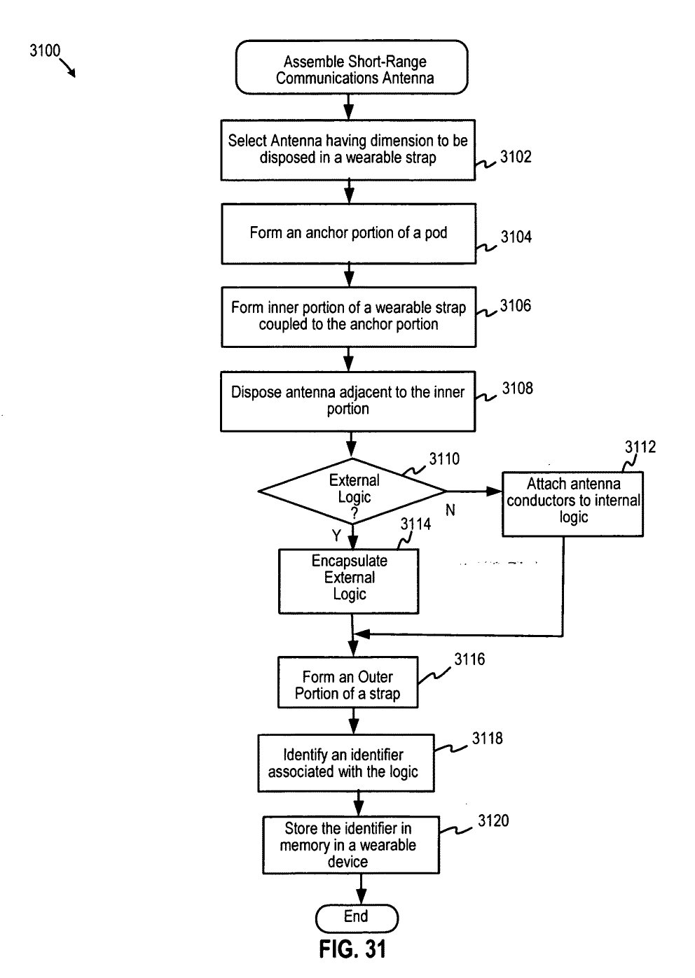

FIG. 32 is a diagram depicting examples of an electrode-based wire bus for facilitating physiological characteristic sensing, according to some embodiments. Electrode or wire bus wire bus 3200, and components coupled with the electrode bus 3200 are depicted. Electrode wire bus 3200 may include a bus substrate 3201 that may be made from a flexible and electrically non-conductive material including but not limited to a thermoplastic elastomer and rubber, for example. In one example, the elastomer material can include, for example, TPE or TPU, to form a flexible substrate in which Kevlar™-based conductors are encapsulated. In one example, the flexible bus substrate 3201 is formed of TPE and has a hardness of approximately 85 to 95 Shore A (e.g., approximately 90 Shore A ins some cases). A side view of bus substrate 3201 illustrates a wire 3212 encapsulated between an upper surface 3201a and a lower surface 3201b of the bus substrate 3201. Wire 3212 may be coupled 3207 with a pad 3203 (shown in dashed line) and pad 3203 may be coupled with an electrode 3202. Electrode 3202 may be coupled with a skirt 3204 and may include a pin 3206 that is positioned in an aperture 3205 of the pad 3203. A dimension of the pin 3206 may be selected to be slightly greater than a diameter of the aperture 3205 so that when the pin 3206 is inserted into the aperture 3205 a press fit is established between the pin 3206 and aperture 3205. Press fitting the pin 3206 into the pad 3203 may provide for a pressure fit that retains the electrode 3202 in contact with the pad 3203 and may also provide for a low electrical resistance connection between the electrode 3202 and pad 3203. The press fit may also be operative to securely couple the skirt 3204, pad 3203 and electrode with one another. Crimping, soldering, or other techniques may be used to couple the pad 3203 and the electrode 3202 with each other, and the press fit is one non-limiting example of how the pad 3203 and electrode may be coupled with each other. A portion of pin 3206 may extend outward of the lower surface 3201b of the bus substrate 3201. After the wire bus 3200 has been fabricated (e.g., by injection molding) the exposed portion of the pin 3206 may be used for electrical continuity testing of one or more of the pad 3203, the electrode 3204, and wire 3212.

Wire 3212 may be connected with a portion of pad 3203 using soldering, crimping, wrapping, or welding for example. As one example, wire 3212 may be laser welded to a portion of pad 3203. Pad 3203, the electrode 3202 or both may be made from an electrically conductive material including but not limited to a metal, a metal alloy, copper, gold, silver, platinum, aluminum, stainless steel, and alloys of those metals. As one example, pad 3203 may be a copper (Cu) washer. Wire 3212 may include insulation 3213 that may be stripped to expose a conductor 3214 that may be connected with the pad 3203. Wire 3212 may be routed along a path in the wire bus 3200 and may exit the wire bus 3200 at a distal end 3209. A portion of the wire 3212 positioned at the distal end 3209 may be stripped to expose conductor 3214 and the conductor 3214 may be tinned (e.g., with solder) in preparation for connecting the conductor 3214 with another structure, such as an electrical node, printed circuit board (PCB) trace, or circuitry, for example. A portion of the wire 3212 positioned at the distal end 3209 may be dressed for subsequent connection with other structures. There may be more electrodes 3202, pads 3203, skirts 3204 and wires 3212 than depicted as denoted by 3221 and 3223.

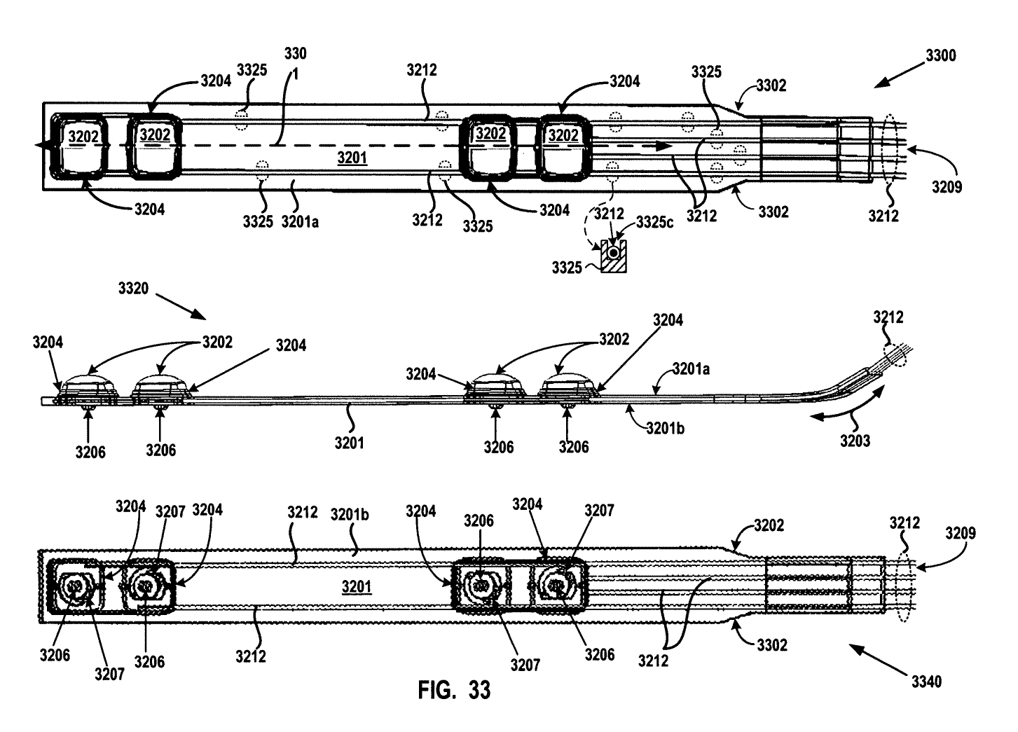

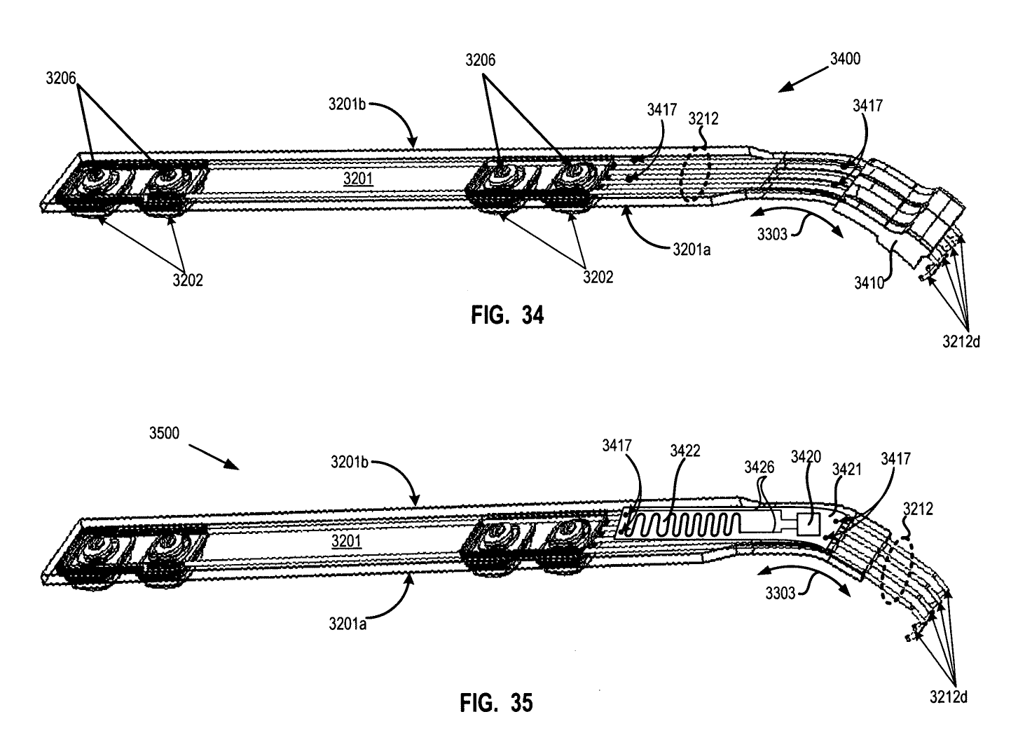

Bus substrate 3201 may include alignment structures (e.g., see 3407 in FIGS. 34 and 35) that may be used to mount other components to the wire bus 3200, such as an antenna and a near field communication chip, for example. Bus substrate 3201 may include a thickness t that may be 1 mm or less in thickness. The material used for bus substrate 3201 may be selected to sustain a continuous pull load of about 2 kg and to sustain a maximum pull load of about 8 kg. Actual force loads may be application dependent and the foregoing are non-limiting examples.

In example 3240, electrode 3202 and skirt 3204 may be positioned relative to an aperture 3241 of an inner strap of a strap band (not shown). A material 3243, such as a material used to form an outer strap of the strap band (e.g., via injection molding). Wire bus 3200, skirt 3204, or structures in a mold may include channels, ports, or other structures configured to provide a path for material 3243 to enter into aperture 3241. From left to right in example 3240, material 3243 (e.g., a thermoplastic elastomer) enters into aperture 3241, fills the aperture 3241 and connects with skirt 3204 along an interface 3245. Skirt 3204 may be made from a material that interfaces with material 3243 to establish a seal between the skirt 3204 and the aperture 3241. A temperature of material 3243 may be operative to heat skirt 3204 and the heat may be operative to form a seal between the electrode 3202 and skirt 3204, skirt 3204 and aperture 3241 or both. Material 3243 may not interface with the electrode 3202 (e.g., a metal material for electrode 3202) and skirt 3204 may be operative as a material that interfaces with electrode 3202 and with material 3243. In some embodiments, skirt 3204 may be made from in interface material configured to integrate electrodes 3203 with a material used to form a strap band, a band, or the like. According to some examples, skirt 3204 may be composed of a polycarbonate material or like material. In some examples, skirt 3204 may expand in dimension when contacted by material 3243 or heat in material 3243 as denoted by 3204e.

In example 3250, electrode 3202 may include a pin 3206 and skirt 3204 may include an aperture 3204a through which the pin 3206 may be inserted. A mold in which the wire bus 3200 is molded or a jig may include a support structure 3230 having a post 3231 upon which the pad 3203 is mounted. Wire 3212 (e.g., stripped to expose conductor 3214) may be connected with the pad 3203 by soldering, crimping, wire wrapping, welding, or by application of an electrically conductive adhesive or epoxy, for example. A material for the bus substrate 3201 may be formed over the pad 3203 and wire 3212. Post 3231 may prevent the material from entering into the aperture 3205 of the pad 3203 so that in a subsequent processing step, pin 3206 of electrode 3202 and skirt 3204 may be connected with the pad 3203. As described above, a pressure or friction fit may be used to connect the pad 3203 with the pin 3206 of the electrode 3202.

Examples 3212a-3212d depict various configurations for wire 3212. In example 3212a, wire 3212 may include a conductor 3214 surrounded by an insulator 3213. In example 3212b, wire 3212 may include a conductor 3214 surrounded by an insulator 3213 and the conductor 3214 surrounding a core 3215 (e.g., a concentrically positioned core). Core 3215 may be made from a high strength material such as a composite, Kevlar, fibers, carbon fiber, or the like, for example. Core 3215 may be electrically conducting or electrically non-conducting. Core 3215 may be used to structurally strengthen wire 3212 against forces that may be caused by stretching wire bus 3200 or a strap band that includes the wire bus 3200. In example 3212c, wire 3212, sans insulation 3213, may include the conductor 3214 surrounding the core 3215. In example 3212d, wire 3212 may include a conductor 3214 (e.g., sans insulation 3213 and core 3215).

FIG. illustrates examples of a top, side, and bottom plan views of an electrode or wire bus, according to some examples. In a top view 3300, electrodes 3202 may be positioned on bus substrate in alignment with an axis 3301. There may be more or fewer electrodes 3202 disposed on bus substrate 3201 than depicted and those electrodes 3202 may be positioned in alignment with each other or some or all of the electrodes 3202 may not be aligned with one another. Bus substrate 3201 may have a different shape than depicted. For example, bus substrate 3201 may have a taper 3302 in its width. Wires 3212 may be routed along a path in the bus substrate 3201. The path may be determined by one or more wire guides 3325 (depicted in dashed line) positioned in a mold or jig (not shown) that may be used to form the wire bus 3200. Wire guide 3325 may include a slot or channel 3325c in which a portion of the wire 3212 may be positioned. Side portions of electrodes 3202 may be coupled with the skirt 3204.

In a side view 3320, a portion of the pins 3206 of electrodes 3202 may extend outward of lower surface 3201b of bus substrate 3201. In other examples the pins may not extend outward of lower surface 3201b or may be cut, trimmed, grounded down or otherwise machined to be flush with or inset from lower surface 3201b. Wire bus 3200 may be formed from a material and may include components (e.g., core-reinforced wires) configured to allow flexing, pulling, stretching, twisting of the wire bus 3200 as denoted by 3303. The material for bus substrate 3201 and its associated components may be selected to withstand a range of torsional loads that may be applied to the wire bus 3200 and/or strap bands the wire bus 3200 is positioned in.

In a bottom view 3340, wires 3212 may be coupled 3207 with their respective pads 3203 and the pads 3203 may include a connection portion configured to receive the wire 3212. Pads 3203 may also include a flat (as will be described below) that allows one of the wires 3212 to be routed past the pad 3203 to another pad 3203.

FIG. 34 is a diagram depicting an example of a can electrode-wire bus implementing a wire bridge, according to some examples. As shown, wire bridge 3410 may be operative to position wires 3212 for attachment to another structure (e.g., internal to a wearable pod) and may also be used to dress the wires 3212 into a configuration for connection to another structure. A surface of the wire bus 3200 may include one or more structures 3417 configured to receive a component to be connected with the wire bus 3200. Structures 3417 may include a post, a pillar, a notch, a groove, etc., for example.

FIG. 35 is a diagram depicting an example of a surface of a wire bus including a substrate in which an antenna is formed, according to some examples. As shown, surface 3201b of the wire bus 3200 includes a substrate 3421 having an antenna 3422 coupled 3426 to, for example, a near field communication (“NFC”) semiconductor device or chip 3420. Substrate 3421 may be a flexible substrate that may be mounted to wire bus 100 using the structures 3417. For example, structure 3417 may be posts and substrate 3421 may include apertures that match positions with and mate with structures 3417. Other components may be coupled with wire bus 3200 and the above mentioned substrate 3421 is a non-limiting example. Components coupled with wire bus 3200 may include wires or other types of interconnect structures that may be connected with other components.

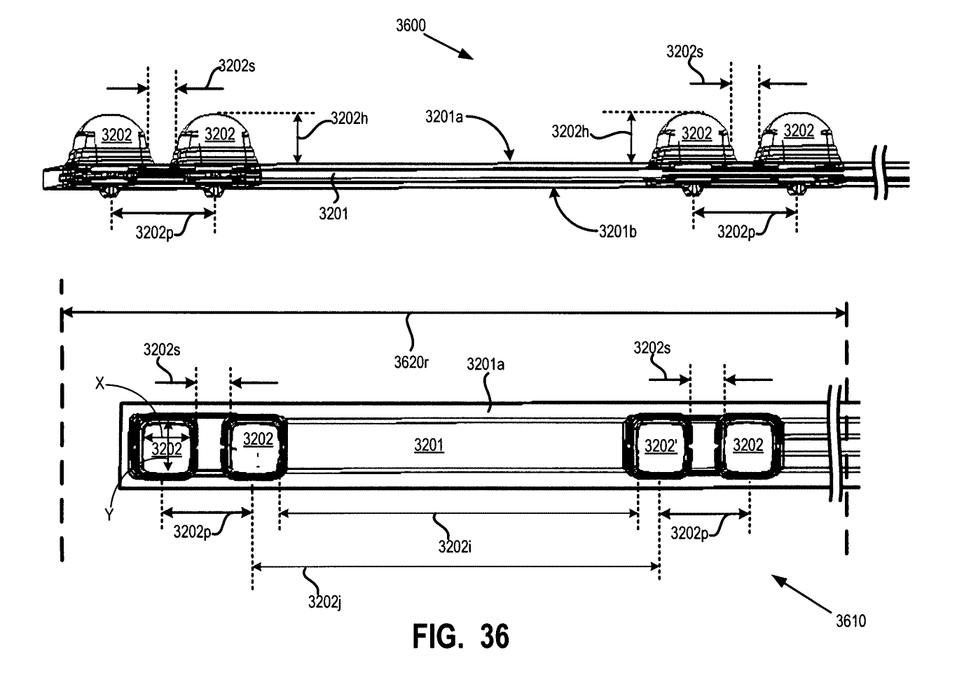

FIG. 36 is a diagram depicting examples of relative spacing and dimensions of electrodes disposed in a wire bus, according to some embodiments. In example 3600, in a side view of wire bus 3200, electrodes 3202 may be positioned relative to one another by a spacing 3202s. In other examples, electrodes 3202 may be spaced apart from one another by a pitch 3202p (e.g., as measured between centers of pins 3206). Electrodes 3202 may have a height 3202h (e.g., as measured from upper surface 3201a to an uppermost surface of the electrode 3202). In example 3610, pairs of adjacent electrodes 3202 may be spaced apart by spacing 3202s or pitch 3202p, and an inner most electrodes 3202′ in each pair may be spaced apart by a distance 3202i or a pitch 3202j (e.g. as measured between pin 3206 centers of electrodes 3202′). Spacing 3202s, 3202i and/or pitches 3202p, 3202j may be selected to position the electrodes 3202 within a target range 3620r, when the wire bus 3200 is included in a system or device that positions the electrodes into contact with a surface of a body portion, such as skin on a wrist, arm, leg, neck, torso, etc. As one example, target range 3620r may be determined by a range of sizes for human wrists ranging from skinny wrists having a small circumference and large wrists having a larger circumference. Although there may be some outlier wrist sizes above and below the target range 3620r, the target range 3620r may be selected to capture wrist sizes for a majority of a population of users. Target range 3620r may encompass a portion of a circumference of those wrists that is positioned on a bottom side of the wrists, for example. Electrodes 3202 positioned within the target range 3620r may be positioned to sense or otherwise detect structures or properties on the skin or beneath the skin (e.g., subcutaneous). For example, subcutaneous structures may include blood vessels or other tissues associated with the sympathetic nervous system (SNS); whereas, skin conductance may be a property measured by contact of electrodes with a surface of the skin. In some examples, electrodes 3202 may be components of a biometric sensor system, such as one that senses bioimpedance (BI). Wire bus 3200 may be positioned in a strap band that is mounted to a wrist or other body portion, and as that strap band shifts its position relative to the body portion, the electrodes 3202 may be positioned within the target range 3620r such that reliable signals may be received from electrodes 3202.

Electrode height 3202h may be selected to provide sufficient contact pressure between the electrode 3202 and a skin surface the electrode 3202 is brought into contact with when the strap band or other device that carriers the wire bus 3200 is mounted to a body portion, such as an arm or wrist for example. As will be described below, an upper surface of electrode 3202 may include a surface area (e.g., X*Y) operative to minimize contact resistance between the electrode 3202 and a skin surface it is placed into contact with and/or to improve a signal-to-noise ratio (S/N) of signals generated by the electrode 3202. The upper surface of the electrode 3202 may have an arcuate shape configured to provide comfort when the electrode 3202 is engaged with the body portion and/or to increase surface area of the electrode 3202.

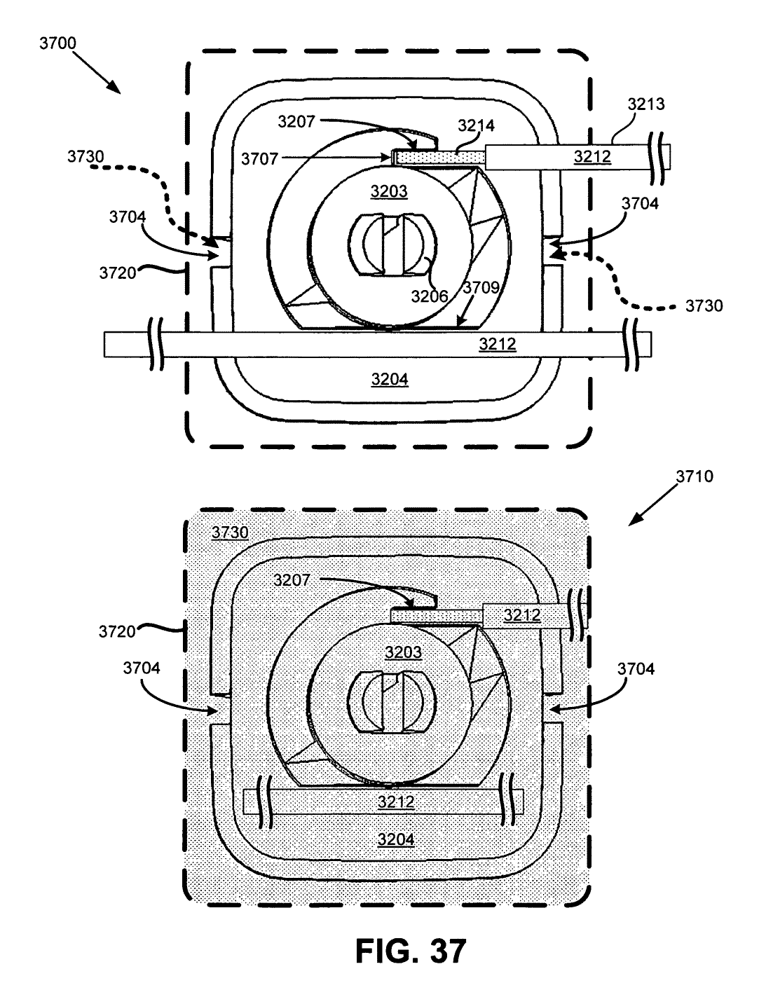

FIG. 37 illustrates examples of wire routing configurations and connection to conductive pads formed in a wire bus, according to some embodiments. In example 3700 a back view of skirt 3204 and pad 3203 includes, for example, a flat 3709 formed in the pad 3203 and operative to allow wire 3212 to be routed pass the pad 3203 for connection with another pad 3203, for example. Pad 3203 may include a notch 3707 where a stripped end of wire 3212 may be positioned for connection of the wire 3212 with the pad 3203. Skirt 3204 may include one or more shot channels 3704. Wire bus 3200 may be positioned in another structure, such as an inner strap band that includes an aperture denoted by dashed line 3720 through which the electrode 3202 and its skirt 3204 may be disposed. A material may be introduced (e.g., injected as part of an injection molding process) into the shot channels 3704 and flow into voids or spaces in the pad 3203, skirt 3204, pad 3203, and into aperture 3720. In example 3710, the material 3730 may fill in and seal a space between the aperture 3720 and the skirt 3204. The material 3730 may also encapsulate structures in the wire bus 3200, such as wires 3212, portions of pad 3203, and portions of skirt 3204, for example. Material 3730 may be introduced into shot channels 3703 via one or more shot ports formed in the wire bus 3200 and aligned with shot channels 3704 during an injection molding process, for example.

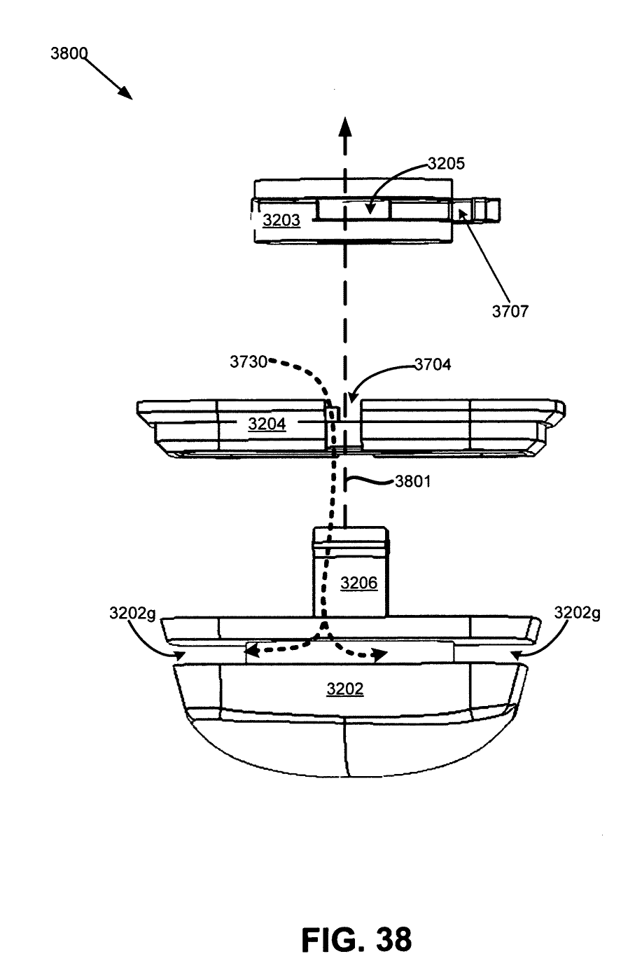

FIG. 38 is a side view 3800 illustrates one example of an electrode and related structures, according to some embodiments. As shown, electrode-wire bus 3200 may include an electrode 3202, a skirt 3204, and a pad 3203, among other things. Pin 3206 of electrode 3202 may be inserted 3801 into an aperture 3204a of skirt 3204 and then into aperture 3205 of pad 3203. Electrode 3202 may include a grooved portion 3202g that is positioned in contact with the skirt 3204 and may open into one of the shot channels 3704. The material 3730 (e.g., a thermoplastic elastomer) may flow through shot channels 3704 and a portion of the material 3730 may flow into grooved portion 3202g as well as into other portions (e.g., aperture 3720) as was described above.

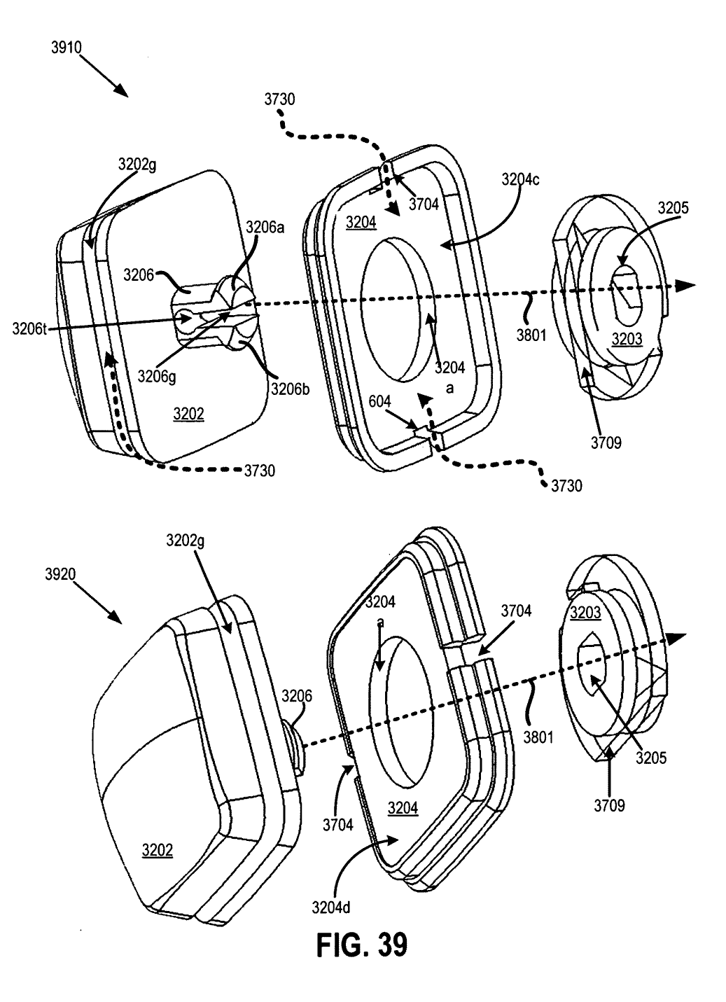

FIG. 39 illustrates profile views 3910 and 3920 of other examples of electrode structures, according to some embodiments. As shown, an electrode structure may include an electrode 3202, a skirt 3204, and a pad 3203 formed in wire bus 3200. Pin 3206 of electrode 3202 may include a slot 3206g that may divide a portion of the pin 3206 into sides 3206a and 3206b. Pin 3206 may be inserted 3801 through aperture 3204a and into aperture 3205 of pad 3203. Insertion through aperture 3205 may cause sides 3206a and 3206b to deflect inward toward each other and then expand outward away from each other upon exiting the aperture 3205. The outward expansion of sides 3206a and 3206b may exert force against walls of aperture 3205 and provide a press fit or friction fit between the pad 3203 and the pin 3206, such that the electrode 3202 is securely coupled with pad 3203. The press fit or friction fit may also be operative to securely couple the skirt 3204 between the pad 3203 and the electrode 3202. Front and rear sides of skirt 3204 may include recessed portions 3204c and 3204d. Material 3730 may flow into recessed portions 3204c and 3204d, groove 3202g, and into pad 3203 via opening 3206t in pin 3206.

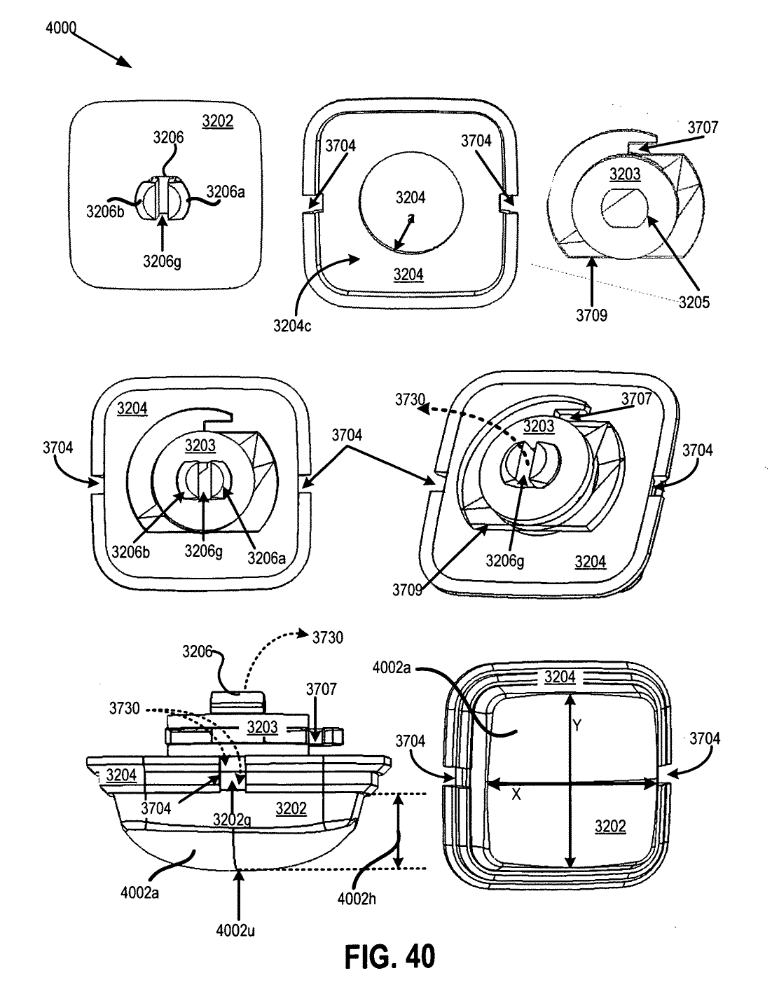

FIG. 40 illustrates views of yet other examples of an electrode structure, according to some embodiments. For example, electrode structure can include electrode 3202, a skirt 3204, and a pad 3203 that may be included in a wire bus 3200 are depicted. An uppermost portion 4002u of electrode 3202 may have a height 4002h (e.g., as measured from a top of the skirt or from surface 3201a of bus substrate 3201) that may be in a range from about 1.0 mm to about 2.5 mm. Height 4002h may be determined in part by a thickness of an aperture (e.g., 3720) that surrounds the electrode 3202 when wire bus 3200 is positioned in another structure, such as an inner strap, for example. If a material that forms the aperture is thick (e.g., 2.0 mm thick), then height 4002h may be higher than would be the case if the material that forms the aperture is thin (e.g., 1.0 mm thick). In some examples, height 4002h may vary among the electrodes 3202. For example, one electrode 3202 may have a height 4002h of approximately 1.5 mm and another electrode 3202 may have a height 4002h of approximately 1.7 mm.

A surface area 4002a of electrode 3202 may be in a range from about 8.0 mm2 to about 20 mm2. For example, surface 4002a may have a dimension of about 4.0 mm in a X-dimension and about 4.00 mm in a Y-dimension for an area of about 16 mm2. Area for surface 4002a may be selected to provide a desired signal-to-noise ratio (S/N) in circuitry coupled with electrode 3202 (e.g., via wire 3212).

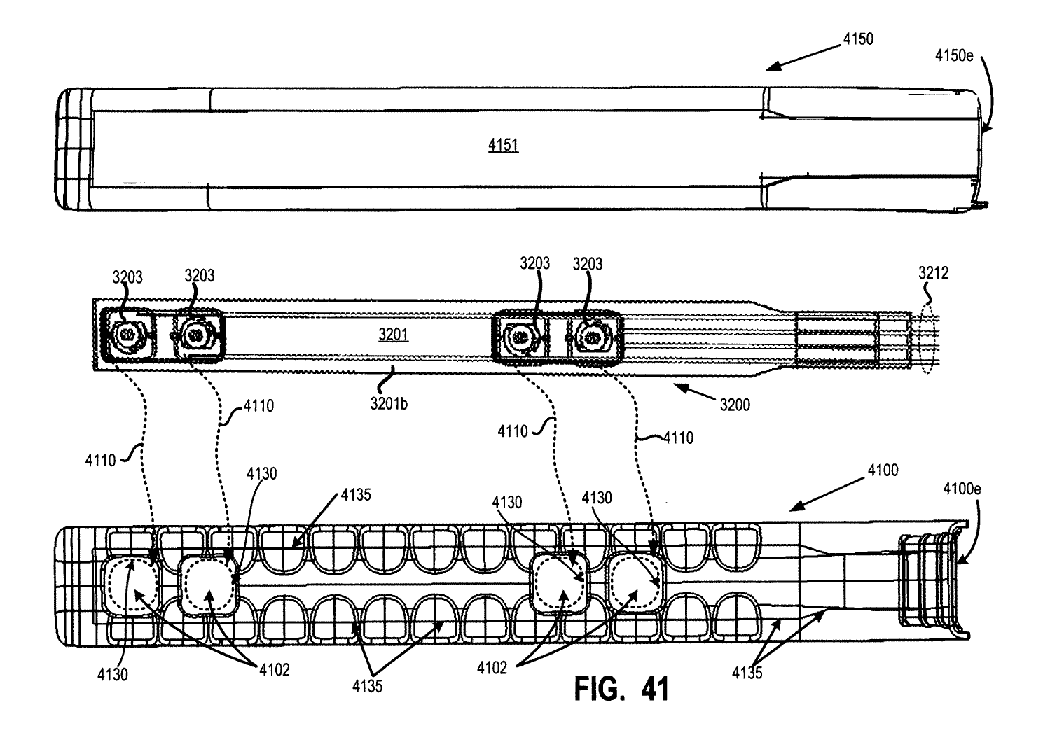

FIG. 41 is a diagram depicting an example of an assembly of a strap band that including a wire bus, an inner strap, and an outer strap, according to some embodiments. In a rear view, wire bus 3200 may be positioned in a previously fabricated lower strap 4100. Inner strap 4100 may include a portion 4100e configured to connect inner strap 4100 with another structure or component. Inner strap 4100 may be connected with another structure or component as part of the previous fabrication. Inner strap 4100 may include apertures 4102 formed in the inner strap 4100 during the previous fabrication. Wire bus 3200 may be moved 4110 into position in inner strap 4100 with its electrodes 3202 and skirts 3204 aligned with apertures 4102 as denoted by dashed lines 4130 which represent an outline of a desired alignment with the apertures 4102 when the wire bus 3200 is positioned in the inner strap 4100. Inner strap 4100 may include a cavity 4135 formed during the previous fabrication and configured to receive the wire bus 3200. Cavity 4135 may mirror an outline of an outer perimeter of the wire bus 3200. The outer strap 4150 will be formed over the connected wire bus and lower strap in a subsequent processing step as will be described below. Outer strap 4150 may also include a portion 4150e configured to connect outer strap 4150 with another structure or component. Portions of surface 3201a of bus substrate 3201 may include a glue, adhesive or the like applied to surface 3201a and operative to facilitate connecting wire bus 3200 with inner strap 4100 (e.g., connecting bus substrate with cavity 4135.

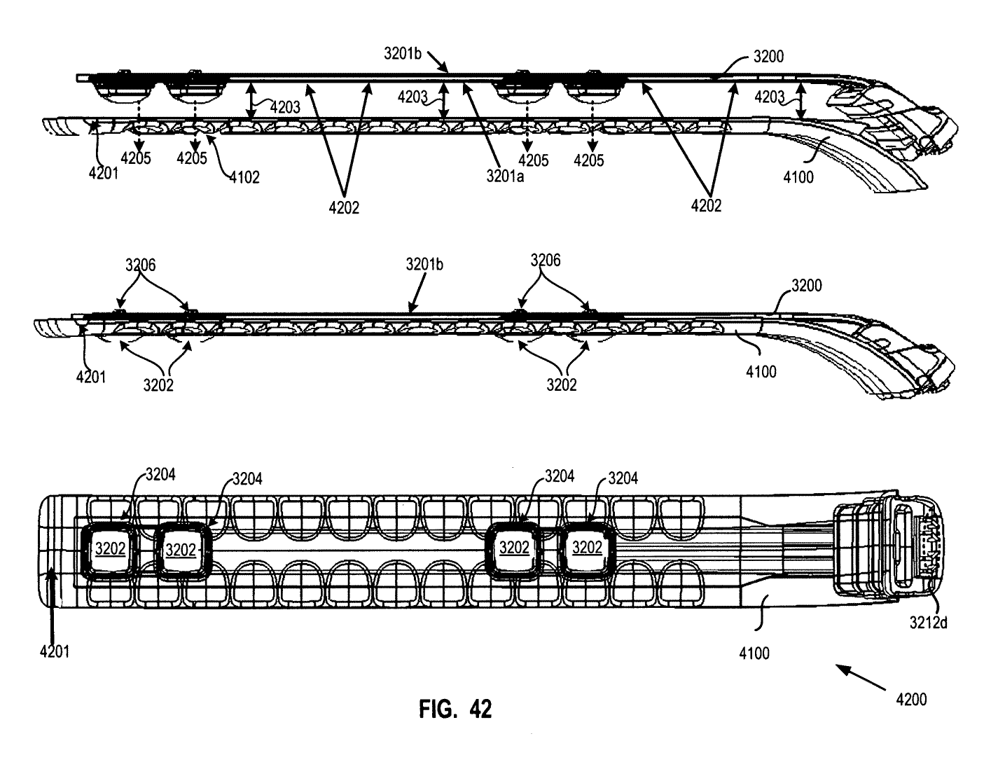

FIG. 42 illustrates an example of a wire bus coupled to an inner strap or inner strap portion, according to some embodiments. Portions 4202 of surface 3201a of bus substrate 3201 may have an adhesive or glue applied to surface 3201a, for example, a pressure sensitive adhesive tape may be applied to one or more portions 4202 of surface 3201a. Wire bus 3200 and inner strap 4100 may be brought into contact 4103 with each other with electrodes 3202 and skirts 3204 aligned 4205 with apertures 4102 as described in reference to FIG. 41. After wire bus 3200 is connected with inner strap 4100, electrodes 3202 extend outward of their respective apertures 4102, and the wire bus 3200 and the inner strap 4100 form sub-assembly 4200.

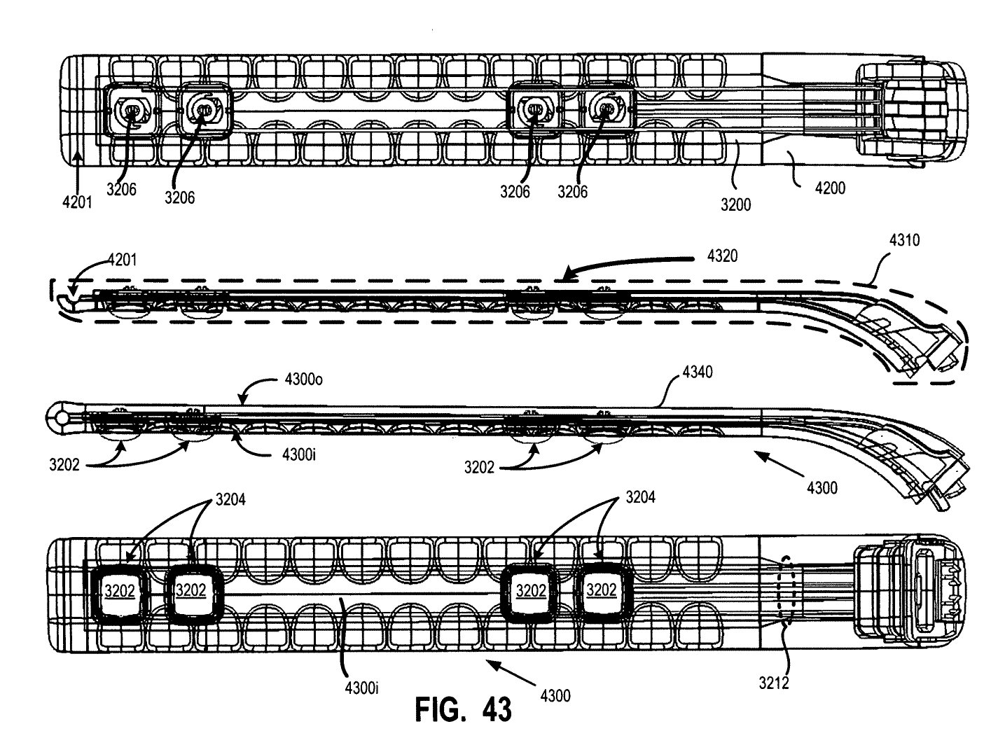

In FIG. 43 one example of an outer strap 4340 being formed on sub-assembly 4200 (e.g., wire bus 3200 coupled with an inner strap 4100) is depicted. Sub-assembly 4200 may be positioned in a mold 4310 including features for an outer strap 4340. A material 4320 (e.g., 3730) such as a thermoplastic elastomer may be injected into mold 4310 to form the outer strap 4340 around the sub-assembly 4200. The outer strap 4340 and inner strap 4200 may form a strap band 4300 that includes portions of the wire bus 3200 encapsulated in the strap band 4300 (e.g., the electrodes 3202 extend outward of inner strap 4200). The material 4320 may flow into shot channels 3704 of skirts 3204 and may seal apertures 4102 as was described above. Inner strap 4200 and outer strap 4340 may be integral with one another after the molding process, such that there may be no visible demarcation of where the inner strap 4200 interfaces with the outer strap 4340. Materials for the inner and outer straps may be the same materials or different materials. Materials for the inner and outer straps may have different colors and may have different surface features or ornamentation. As shown in FIG. 43, a strap band 4300 may be formed with a thermoplastic elastomer material 4320 encapsulating the circuitry therein (e.g., an electrode-wire bus, an NFC chip, an antenna, etc.) to protect it from environmental conditions. Further, interface materials of the skirt and the anchor portions (e.g., as described herein) ensure that elastomer material 4320 bonds/integrates with metal (e.g., stainless steel electrodes and magnesium cradle in the wearable pod).

A system may include one or more strap bands, with one of the strap bands being configured as strap band 4300 and another of the strap bands not including the wire bus 3200. The system may include two strap bands 4300 with each strap band 4300 having its own encapsulated wire bus 3200 and associated wires 3212, pads 3203, electrodes 3202, and skirts 3204, for example. The number and placement of electrodes 3202 in the two strap bands 4300 may be the same or different (e.g., one strap band 4300 may have four electrodes 3202 and the other strap band 4300 may have two electrodes 3202). Each strap band in the system may include fastening hardware (e.g., a buckle, a clasp, a latch, etc.) configured to couple the two strap bands with each other and/or to mount the two strap bands to a structure, such as a portion of a human body, such as the arm, the wrist, the leg, the torso, the neck, etc., for example. A system may include two strap bands with each strap band coupled with a device. For example, distal ends of each strap band in the system may couple with a main module that may include structures (e.g., circuitry, PCB traces, etc.) that couple with wires 3212 positioned at the distal end or one or both of the strap bands.

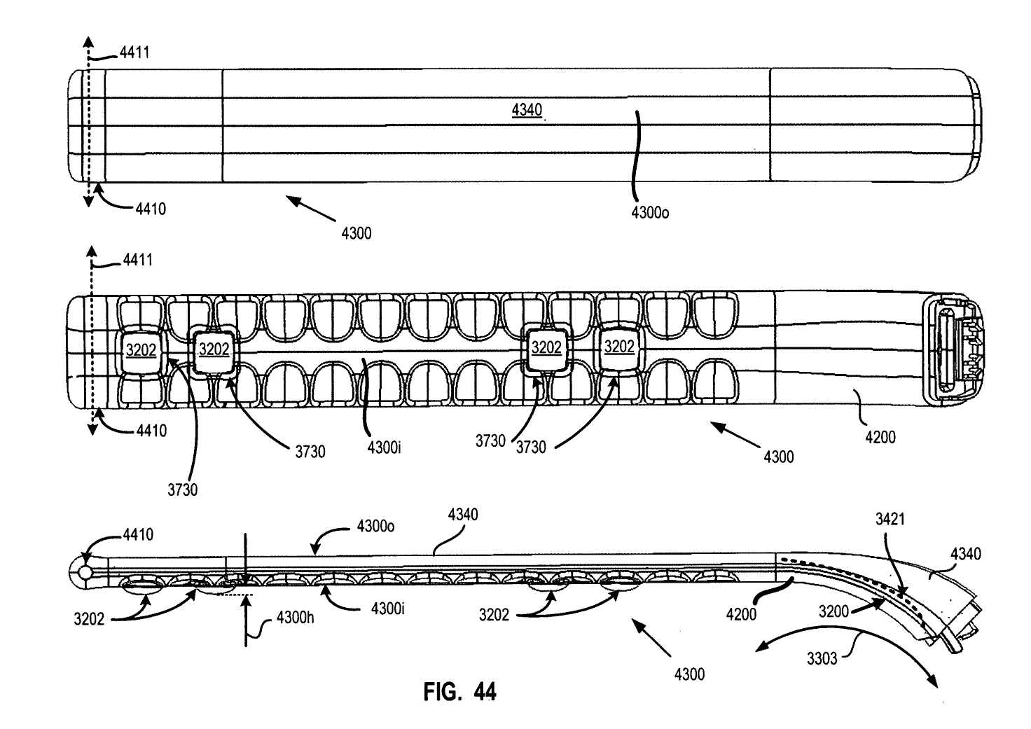

FIG. 44 illustrates top, side and bottom views of one example of a strap band 4300 that includes an encapsulated wire bus 3200 and sealed electrodes 3202. An aperture 4410 configured to accept 4411 fastening hardware for strap band 4300 may be formed by a portion 4201 (e.g., see 4201 in FIGS. 42 and 43) and the molding process of FIG. 43. Strap band 4300 may be flexible 3303 as described above. Moreover, prior to the molding of the outer strap 4340, the substrate 3421 including the antenna 3422 and near field communication chip 3420 may be positioned on the wire bus 3200. Upper surface 4002u of electrodes 3202 (see FIG. 40) may extend above a surface 4300i (e.g., an inner surface) of the inner strap 4200 by a height 4300h in a range from about 1.0 mm to about 2.0 mm, for example. In some examples, height 4300h may vary among the electrodes 3202. For example, one electrode 3202 may have a height 4300h of approximately 0.9 mm and another electrode 3202 may have a height 4300h of approximately 1.2 mm. Subsequent to forming the strap band 4300, a demarcation between the inner strap 4200 and outer strap 4340 may not be discernible (e.g., visually) and the inner and outer straps may appear as a single integrated unit.

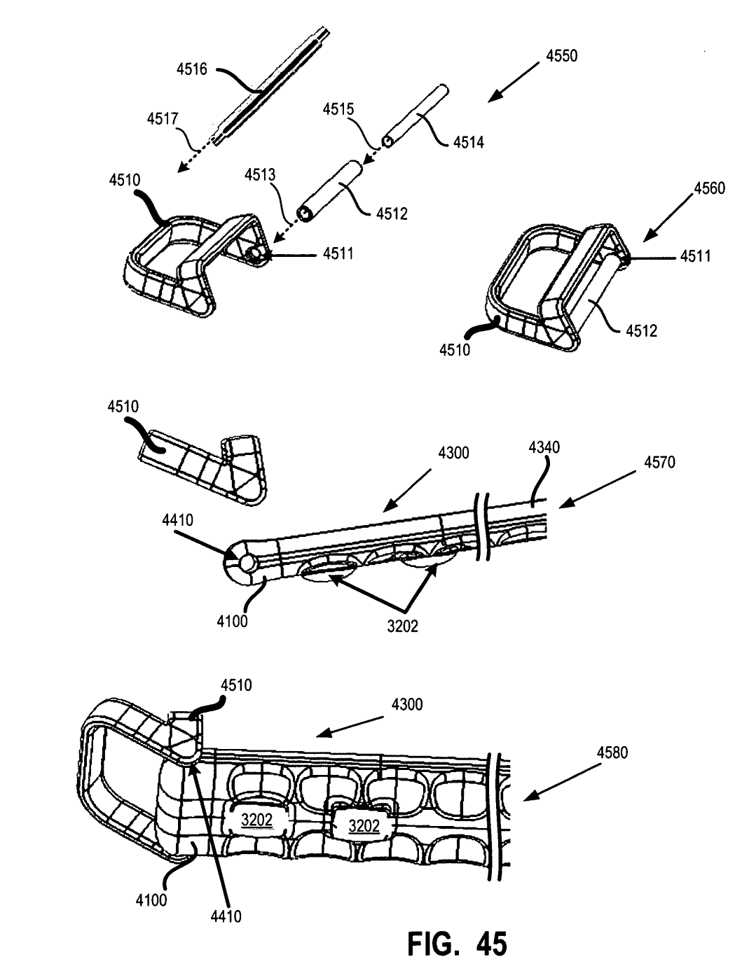

FIG. 45 illustrates examples 4550-4580 of fastening hardware that may be coupled with a strap band 4300. In example 4550 a buckle 4510 may include an aperture 4511 through which a sleeve 4512 may be inserted 4513. A pin 4514 may be inserted 4515 into the sleeve 4512 to secure the buckle 4510 to aperture 4410 in the strap band 4300 as depicted in examples 4560-4580. Pin 4514 may be a spring pin or spring bar 4516 (e.g., like those used with watch bands) that may replace pin 4514, sleeve 4512 or both. Spring pin 4516 may include dimensions configured to allow the spring pin 4516 to be inserted 4517 into aperture 4511 of buckle 4510, or if sleeve 4512 is used, then insertion 4517 into sleeve 4512.

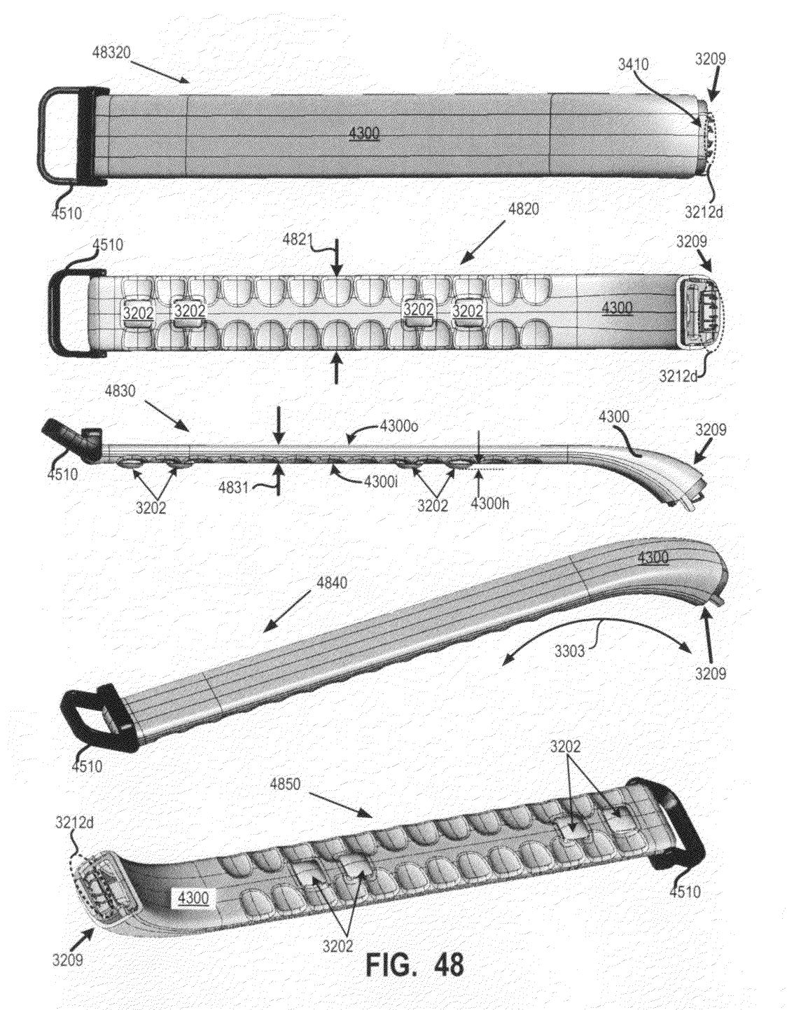

Referring now to FIG. 48, various views 4810-4850 of the strap band 4300 are depicted. Strap band 4300 depicted in views 4810 to 4850 is just one non-limiting example and strap band 4300 may include more of fewer elements than depicted in FIG. 48 and may have an appearance that differs from the examples depicted in FIG. 48. Strap band 4300 may include one or more colors. Strap band 4300 may include one or more surface finishes (e.g., glossy, flat, matte, etc.). Strap band 4300 may be translucent or transparent (e.g., to reveal structure beneath surfaces 4300o and/or 4300i). After strap band 4300 has been fabricated as described above (e.g., in reference to FIGS. 41-45), inner strap 4200 and outer strap 4340 may not be discernible (e.g., visually discernable) and strap band 4300 may appear as a unitary whole (e.g., no visible seems or structures that would indicate strap band 4300 is composed of inner and outer straps). Strap band 4300 may include surface features and/or ornamentation (e.g., for esthetic purposes) on outer surface 4300o and/or inner surface 4300i, for example. Although views 4810-4850 depict dressed wires 3212d, actual configurations for the wires 3212 may be application dependent and are not limited to the exampled depicted herein.

In views 4810-4850, the buckle 4510 is depicted attached to strap band 4300; however, the strap band 4300 need not include the buckle 4510 and the types of fastening hardware that may be coupled with strap band 4300 are not limited to examples depicted herein. Although actual dimensions for strap band 4300 may be application dependent, strap band 4300 may have a width 4821 (see view 4820) in a range from about 8 mm to about 15 mm, for example. In some examples, a width of the strap band 4300 may vary along a length of the strap band 4300. For example, strap band 4300 may be wider at the buckle 4510. Width 4821 may be the smallest width of strap band 4300, for example. A thickness of strap band 4300 may vary along a length of the strap band 4300 (e.g., strap band 4300 may be thicker at distal end 3209); however, notwithstanding the height 4300h of the electrodes 3202 above surface 4300i, strap band 4300 may include a thickness 4831 (see view 4830) in a range from about 0.9 mm to about 3.2 mm, for example. Strap band 4300 may include thickness 4831 along portions of the strap band 4300 that are positioned into contact with a body portion of a user when a device that includes strap band 4300 is worn by the user, such as a portion of an arm adjacent to a wrist of the user. Thickness 4831 may be selected to be the thinnest portion of strap band 4300.

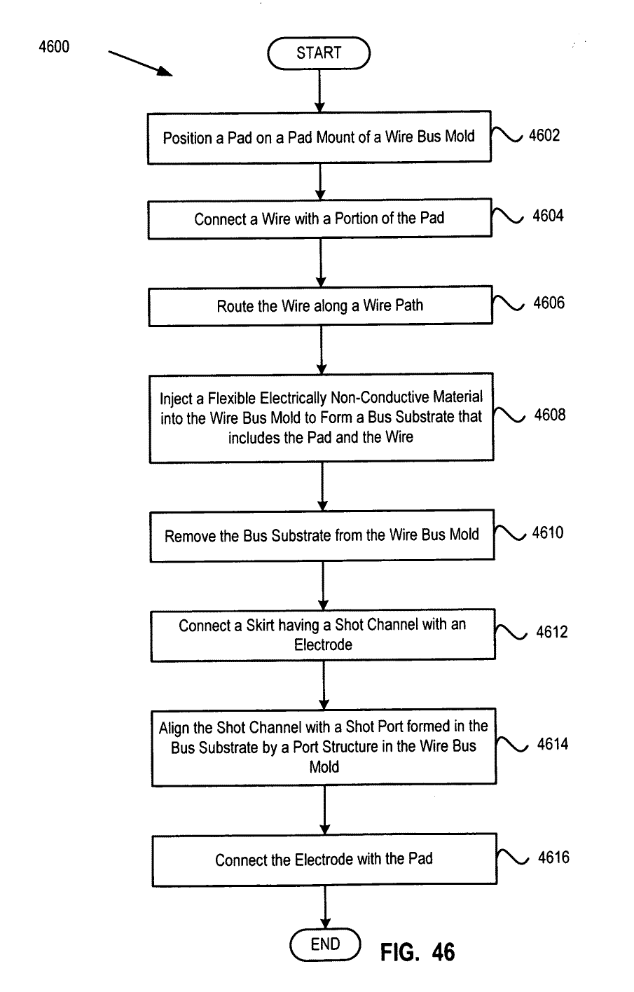

FIG. 46 illustrates one example of a flow diagram 4600 for a method of fabricating a wire bus 3200. At a stage 4602 a pad (e.g., 3203) may be positioned on a pad mount (e.g., 3230) of a wire bus mold. At a stage 4604 a wire (e.g., 3214 of 3212) may be connected with a portion of the pad. At a stage 4606 the wire may be routed along a wire path. At a stage 4608 a flexible electrically non-conductive material (e.g., a thermoplastic elastomer) is injected into the wire bus mold to form a bus substrate (e.g., 3201) that includes one or more pads with each pad having a wire connected to it. At a stage 4610 the bus substrate may be removed from the wire bus mold. At a stage 4612, a skirt (e.g., 3204) having a shot channel (e.g., 3704) may be connected with an electrode (e.g., 3202). At a stage 4614 the shot channels in the skirts may be aligned with a shot port formed in the bus substrate by a port structure in the wire bus mold. At a stage 4616 the electrode may be connected with the pad (e.g., the electrode 3202 with its connected skirt 3204).

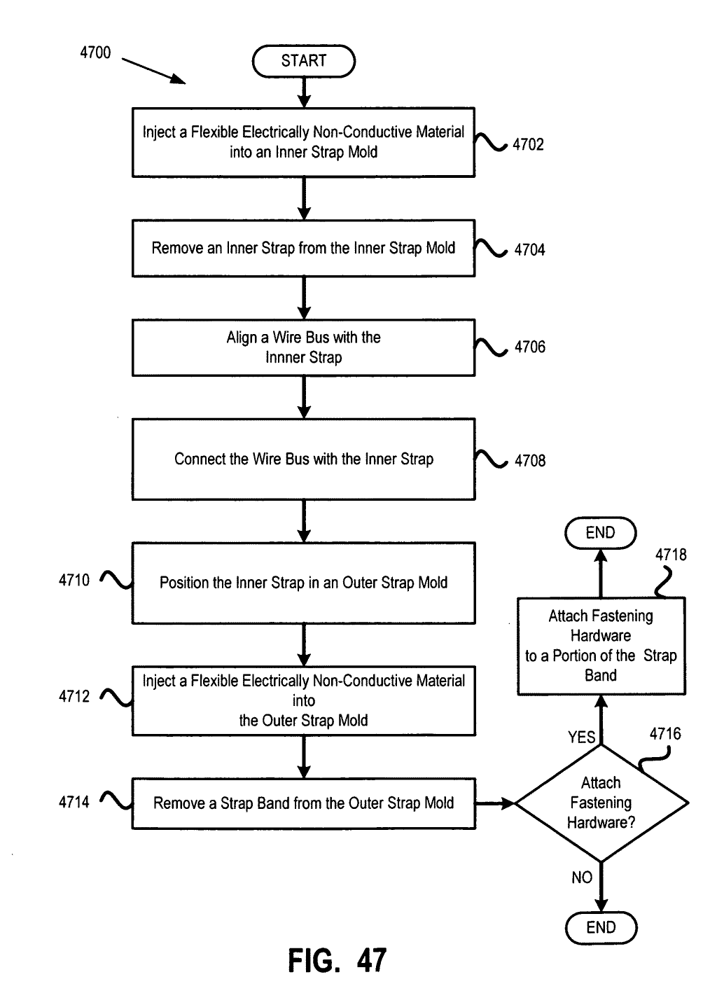

FIG. 47 illustrates one example of a flow diagram 4700 for a method of fabricating a strap band (e.g., 4300) that includes a wire bus 3200. At a stage 4702 a flexible electrically non-conductive material (e.g., a thermoplastic elastomer) may be injected into an inner strap mold. At a stage 4704 an inner strap (e.g., 4100) may be removed from the inner strap mold. At a stage 4706 a wire bus (e.g. 3200) may be aligned with the inner strap. At a stage 4708 the wire bus may be positioned into contact with the inner strap while maintaining alignment between the wire bus and the inner strap. At a stage 4710 the inner strap and its connected wire bus (e.g., sub-assembly 4200) may be positioned in an outer strap band mold. At a stage 4712 a flexible electrically non-conductive material (e.g., a thermoplastic elastomer) may be injected into the outer strap mold. At a stage 4714 a strap band may be removed from the outer strap mold. At a stage 4716 a decision may be made as to whether or not to attach fastening hardware (e.g., 4510, 4512, 4514) to the strap band. If a NO branch is taken, then the flow 4700 may terminate. On the other hand, if a YES branch is taken, then flow 4700 may transition to another stage, such as a stage 4718, for example. At the stage 4718, the fastening hardware is attached to a portion of the strap band.

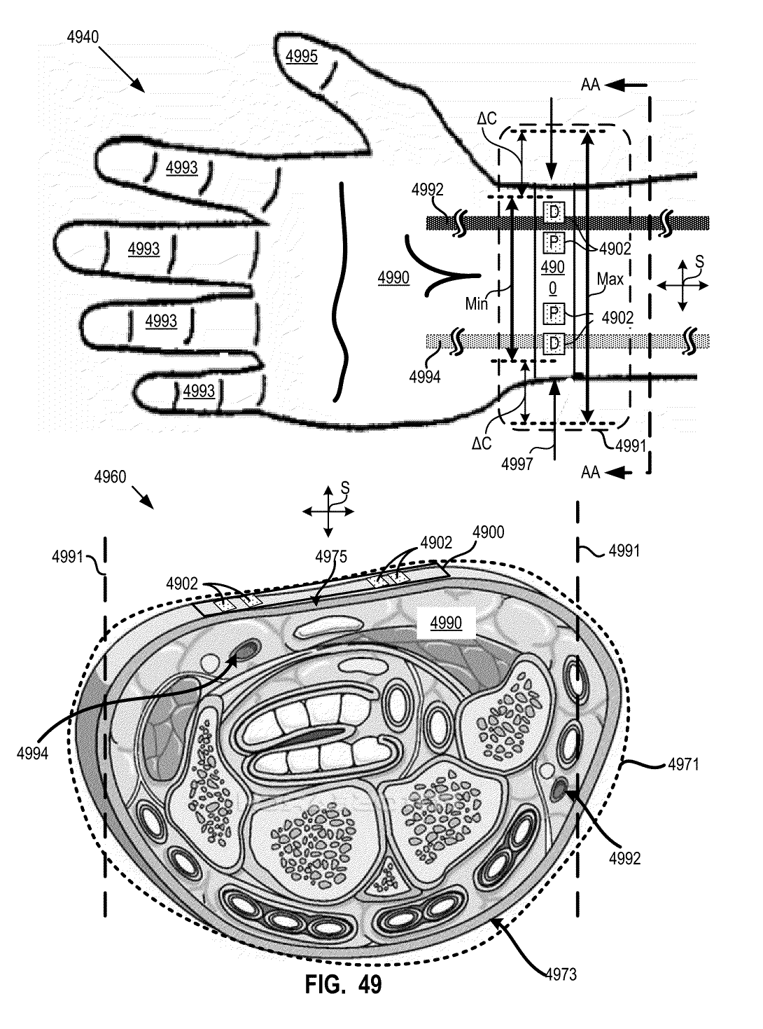

Reference is now made to FIG. 49 where examples 4940 and 4960 of a strap band 4900 positioned on a body portion 4990 are depicted. Here, for purposes of explanation, a non-limiting example of a body portion is a wrist; however, the present application is not limited to a wrist and strap band 4900 may be used with other body portions, including but not limited to the torso, the neck, the head, the arm, the leg, and the ankle, for example.

In example 4940, electrodes 4902 of strap band 4900 may be configured to sense signals, such as biometric signals, from structures of body portion 4990 positioned in a target region 4991. As one non-limiting example, the structure of interest may include the radial artery 4992 and the ulnar artery 4994. The radial artery 4992 is the largest artery that traverses the front of the wrist and is positioned closest to thumb 4995. Ulnar artery 4994 runs along the ulnar nerve (not shown) and is positioned closest to the pinky finger 4993. The radial 4992 and ulnar arteries arch together in the palm of the hand and supply the fingers 4993, thumb 4995 and front of the hand with blood. A heart pulse rate may be detected by blood flow through the radial 4992 and ulnar arteries, and particularly from the radial artery 4992. Accordingly, strap band 4900 and electrodes 4902 may be positioned within the target region 4991 to detect biometric signals associated with the body, such as heart rate, respiration rate, activity in the sympathetic nervous system (SNS) or other biometric data, for example.

Target region 4991 is depicted as being wider than the wrist 4990 and spanning a depth along the wrist 4990 to illustrate that variations in body anatomy among a population of users will result in differences in wrist sizes and some user's may position the strap band 4900 closer to the hand; whereas, other user's may position the strap band 4900 further back from the hand. Now the view in example 4940 is a ventral view of the hand 4990; however, the wrist 4990 has a circumference C that may vary ΔC among users. Arrows 4994 indicate a width of the wrist 4990 for the example 4940; however, in a population of users, circumference (see 4971 of example 4960) of a wrist may vary from a minimum Min (e.g., a very small wrist) to a maximum Max (e.g., a very large wrist). To accommodate variations in wrist circumference ΔC from Min to Max, dimensions of strap band 4900, dimensions of electrodes 4902 and positions of the electrodes 4902 relative to each other and relative to other structures the strap band 4900 may be coupled with, may be selected to position the electrodes 4902 within the target region 4990 for wrist sizes spanning a minimum wrist size of about 135 mm in circumference to a maximum wrist size of about 180 mm in circumference, for example. In other examples, the dimensions and positions may be selected to position the electrodes 4902 within the target region 4990 for wrist sizes spanning a minimum wrist size of about 130 mm in circumference to a maximum wrist size of about 200 mm in circumference. For example, within the target region 4990, electrodes of strap band 4900 may be positioned to sense signals from the radial 4992 and ulnar 4994 arteries for wrist circumferences within the aforementioned 130 mm to 200 mm range, even when the strap band 4900 overlays a flat or curved surface of the wrist 4990 or is displaced to the left, the right, up, or down as denoted by arrow for S on wrist 4990 due to variations in where user's like to place their strap bands on their wrist 4990. Therefore, the strap band 4900 may not require an exact centered location on writs 4990 in order for electrodes 4902 to sense signals from structure in the target region 4991 (e.g., 4992 and 4994).

Some of the electrodes 4902 may have signals applied to them (e.g., are driven) and are denoted as D; whereas, other electrodes 4902 may pick up signals (e.g., receive signals) and are denoted as P. Positioning and sizing of the electrodes 4902 that are adjacent to each other (e.g., a driven D electrode next to a pick-up P electrode) may be selected to prevent those electrodes from contacting each other when the strap band 4900 is bent or otherwise curved when donned by the user. For example, if electrodes 4902 lie on an approximately flat portion of wrist 4990, then adjacent electrodes 4902 (e.g., a D and P) may not be significantly urged inward toward each other because they are lying on an approximately planar surface. On the other hand, if electrodes 4902 lie on a curved portion of wrist 4990, then adjacent electrodes 4902 (e.g., a D and P) may be urged inward toward each other, and if the adjacent electrodes are spaced to close to each other, then their inward deflection might bring them into contact with each other (e.g., they become electrically coupled) and the signal being received by the pick-up P electrode will be the signal being driven on the drive D electrode and not the signal from structure in target region 4991.

Example 4960 illustrates a cross-sectional view of wrist 4990 along a dashed line AA-AA. A circumference of the wrist 4990 is denoted as 4971 and will vary based on wrist size. As depicted, strap band 4900 is positioned on a ventral portion of wrist 4990 in a region 4975 that is relatively flat; however, in the target region 4991, moving left or right away from 4975 towards the boundary of the target region 4991, the surface of wrist 4990 becomes curved. Moreover, wrist 4990 has curvature in a region 4973 of a dorsal portion of the wrist 4990. Although many users will likely wear a device that includes the strap band 4900 in a prescribed manner in which the electrodes 4902 of the strap band 4900 are placed against the bottom of the wrist 4990 (e.g., the ventral portion), some users may prefer to place the strap band 4900 and its electrodes 4902 on the dorsal portion 4973 where the surface of wrist 4990 includes curvature. In either case, strap band dimensions and electrode dimensions and placement may be selected to establish sufficient contact of the electrodes 4902 with skin of the wrist 4990 within the target region 4991 so that signals driven onto drive D electrodes are coupled with wrist 4990 and signals from wrist 4990 are received by pick-up electrodes P.

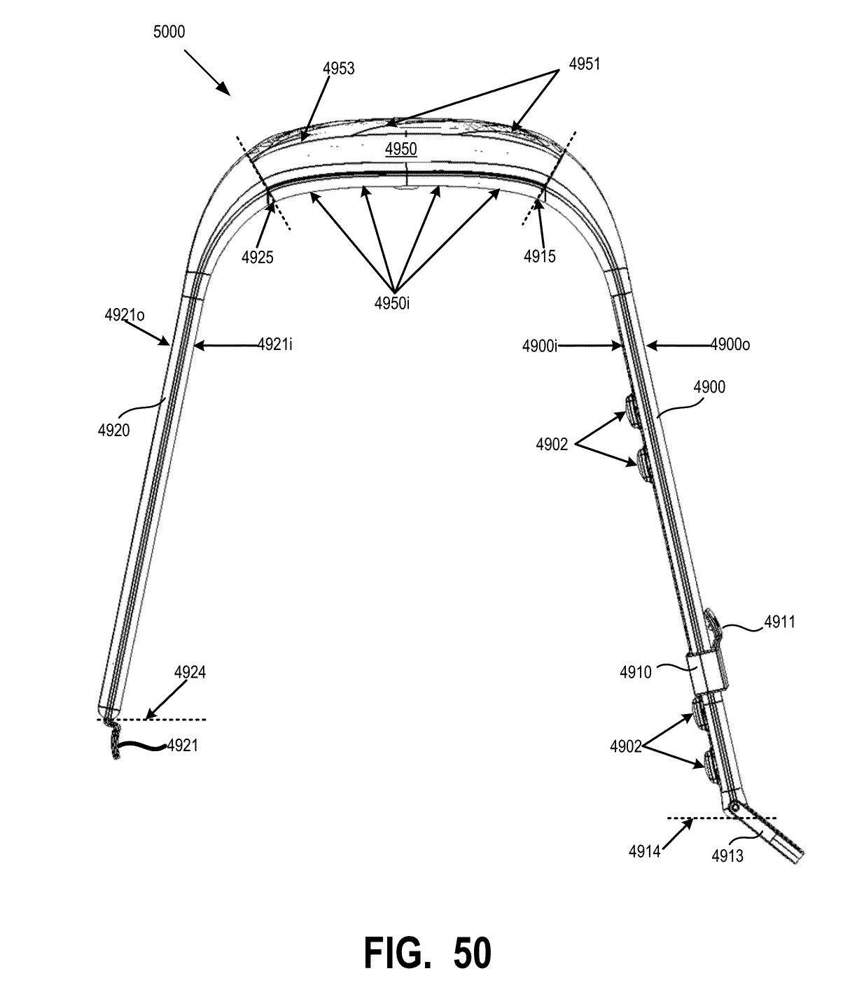

Moving now to FIG. 50 where a side view of a strap band 4900 coupled with a device 4950, such as a wearable pod, is depicted. Here, wearable pod 4950, a band 4920, and strap band 4900 may form a system 5000. Device 4950 may include circuitry, one or more processors (e.g., DSP, μP, μC), memory (e.g., non-volatile memory), data storage (e.g., for algorithms configured to execute on the one or more processors), one or more sensors (e.g., temperature, motion, biometric, ambient light), one or more radios (e.g., Bluetooth—BT, WiFi, near field communications—NFC), circuit boards, a power source, a display (e.g., LED, OLED, LCD), transducers (e.g., a loudspeaker, a microphone, a vibration engine), one or more antennas, a communications interface (e.g., USB), a capacitive touch interface, etc. for example. Device 4950 may include an arcuate inner surface 4950i having a curvature selected to prevent or minimize rotation of system 5000 around wrist 4990 (or other body portion) when system 5000 is donned by a user. Preventing or minimizing rotation of system 5000 may be operative to maintain position of electrodes 4902 within the target region 4991 and/or maintain contact between the electrodes 4902 and skin within the target region 4991. Device 4950 may include ornamentation 4951 (e.g., for esthetic purposes) on an upper surface 4953.

Band 4920 may be a mechanical band, that is, a band configured to couple with strap band 4900 for donning system 5000 on a body portion of a user, such as the wrist 4990 of FIG. 49. Band 4900 may be purely passive (e.g., no electronics disposed in it) or may be active (e.g., includes circuitry and/or passive and/or active electronic components). Band 4920 may include a latch 4921 configured to mechanically couple with a buckle 4910 disposed on strap band 4900. Latch 4921 and a portion of band 4920 may be inserted through a loop 4913 disposed on strap band 4900. Band 4920 may include an inner surface 4920i and an outer surface 4920o. When band 4920 is inserted into loop 4913 and buckle 4910 a portion of inner surface 4920i may contact a portion of an outer surface 4900o of strap band 4900.

Strap band 4900 may include a plurality of electrode 4902 positioned on and extending outward of an inner surface 4900i. Electrodes 4902 and a portion of inner surface 4900i may be positioned in contact with skin in target region 4991 (e.g., skin on wrist 4990) when the system 5000 is donned by a user. In addition to electrodes 4902, strap band 4900 may house other components, such as wires for coupling electrodes 4902 with circuitry, antenna, a power source, circuitry, integrated circuits (IC's), passive electronic components, active electronic components, etc., for example.

Strap band 4900 and band 4920 may couple with device 4950 at attachment points denoted as 4915 and 4925 respectively. For purposes of explanation, attachment points 4915 and 4925 may be used as non-limiting examples of reference points for dimensions described herein. Further, dashed line 4914 on strap band 4900 and dashed line 4924 on band 4920 may be used as non-limiting examples of reference points for dimensions described herein.

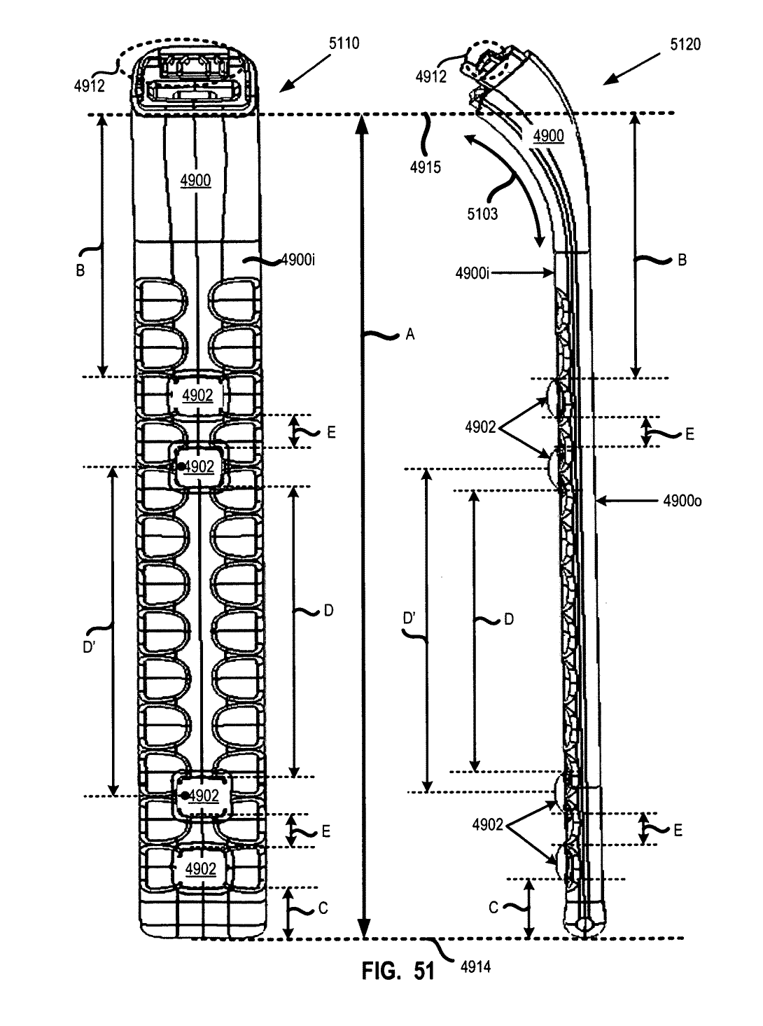

Turning now to FIG. 51 where a top plan view 5110 and a side view 5120 of a strap band 4900 are depicted. In view 5110 (e.g., looking down on inner surface 4900i), dashed line 4915 may serve as a reference point for dimensions A-E. Strap band 4900 may include wires 4912 that exit strap band 4900 proximate its connection point with another structure, such as device 4950 of FIG. 50, for example. Wires 4912 may be coupled with electrodes 4902 and may be coupled with circuitry (e.g., circuitry in device 4950). An overall length of strap band 4900 as measured from line 4915 to line 4914 may be dimension A. Dimension B may be a distance from line 4915 to an edge of electrode 4902. Dimension C may be a distance from line 4914 to an edge of electrode 4902. Dimension D may be a distance between inner facing edges of the two innermost electrodes 4902. Dimension D′ may be a distance between centers of the two innermost electrodes 4902, with distance D′ being greater than the distance D (i.e., D′>D). Dimension E may be a distance between edges of adjacent electrodes 4902.

Dimensions A-E are presented in side view in view 5120. In side view 5120, strap band 4900 may include an arcuate portion as denoted by arrows for 5103. Strap band 4900 may be flexible along its length (e.g., from 4915 to 4914). Although some dimensions other than D′ are measured from edge-to-edge (e.g., dimension E between edges of adjacent electrodes 4902), center-to-center dimensions may also be used and the present application is not limited to edge-to-edge or center-to-center dimensions for measurements described herein. Side view 5120 illustrates electrodes 4902 extending outward of inner surface 4900i of strap band 4900.

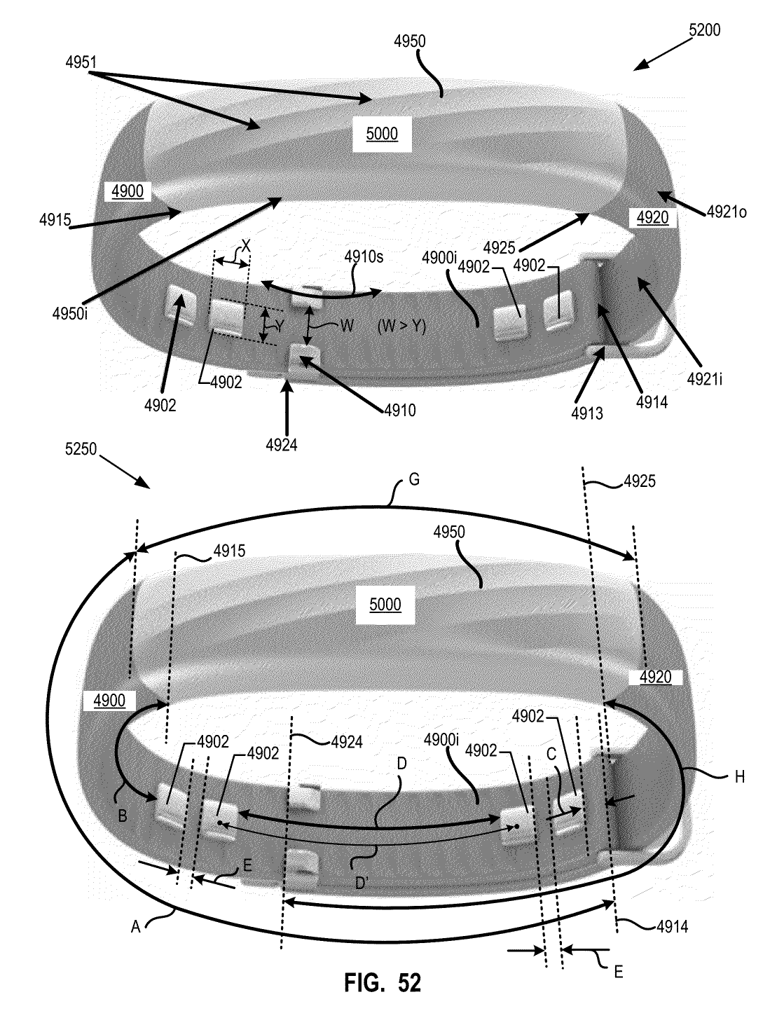

FIG. 52 illustrates profile views 5200 and 5250 of a system 5000 including strap band 4900. Views 5200 and 5250 depict the system 5000 in a configuration the system would have if donned on a user (e.g., system 5000 attached to wrist 4990 of FIG. 49). In view 5200, device 4950 is coupled with band 4920 and strap band 4900 with band 4920 inserted through loop 4913 and latch 4921 coupled with buckle 4910. Electrodes 4902 are depicted positioned along inner surface 4900i and having dimensions X and Y. Buckle 4910 includes a gap having a width dimension W that is greater than the Y dimension of electrodes 4902 (e.g., W>Y), so that sliding 4910s buckle 4910 along the strap band 4900 in the direction of arrows for 4910s will allow the buckle 4910 to slide past the electrodes 4902 without making contact with and without establishing electrical continuity with the electrodes 4902.

Moving to view 5250 where the aforementioned dimensions A-E are depicted along with dimensions for other components of system 5000, namely, dimension G for wearable pod device 4950 and dimension H for band 4920. Dimensions A-E, X, Y, W and G-H may be selected to form a system 5000 that when donned by a user having a body portion circumference (e.g., a circumference of a wrist) in a range from about 130 mm to about 200 mm, will position the electrodes 4902 within the target region 4991 with sufficient contact force with skin in the target region to obtain a high signal-to-noise-ratio for circuitry that receives signals from pick-up electrodes P (e.g., the two innermost electrodes 4902) in response from signals driven onto drive electrodes 4902 (e.g., the two outermost electrodes 4902). Although a range from about 135 mm to about 180 mm may be a typical range of wrist sizes found in a population of users, the larger range of from about 130 mm to about 200 mm may represent outlier ranges that are not typical but nevertheless may occasionally be encountered in a population of users. For example, a very skinny wrist of about 130 mm or a very large wrist of about 200 mm may be corner case exceptions to the more typical range beginning at about 135 mm and ending at about 180 mm of circumference.

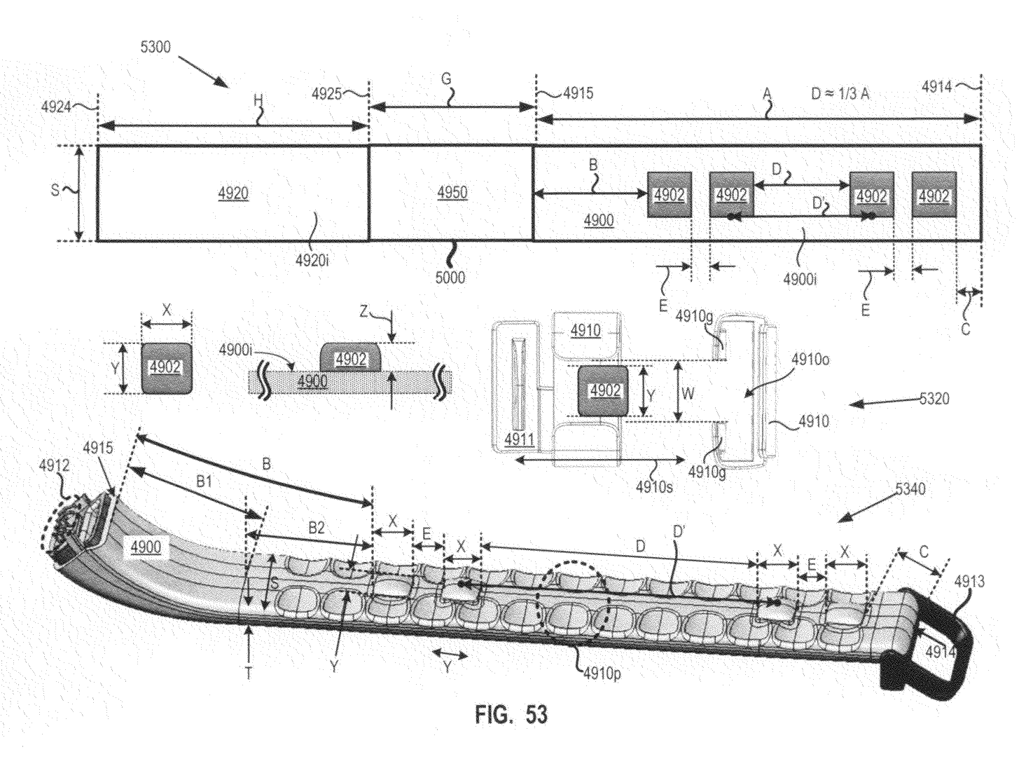

Reference is now made to FIG. 53 where views of strap band 4900 and relative dimensions and positions of components of strap band 4900 are depicted. In view 5300, a system 5000 may include the following example dimensions in millimeters (mm) with an example dimensional tolerance of +/−0.2 mm or less (e.g., +/−0.1 mm): dimension H for band 4920 may be 80.0 mm (e.g., from 4924 to 4925 in FIG. 50); dimension G for device 4950 may be 45.0 mm (e.g., from 4925 to 4915 in FIG. 50); dimension A for strap band 4900 may be 95.0 mm (e.g., from 4915 to 4914 in FIG. 50); dimension B from 4915 to an edge of outermost electrode 4902 may be 32.0 mm; dimension E from an edge of outermost electrode 4902 to an edge of adjacent innermost electrode 4902 may be 4.0 mm; dimension D from an edge of innermost electrode 4902 to an edge of the other innermost electrode 4902 may be 31.5 mm edge-to-edge or dimension D′ for innermost electrodes 4902 may be 36.0 mm center-to-center; distance E from an edge of innermost electrode 4902 to the other outermost electrode 4902 may be 4.0 mm; distance C from an edge of the outermost electrode 4902 to 4914 may be 5.5 mm; and a distance S of band 4920, strap band 4900 or both may be 10 mm-11 mm (e.g., a width of the band 4920 and/or strap band 4900). As one example, distance D may be approximately one-third (⅓) the dimension A for strap band 4900, such that if A=95.0 mm, then D may be approximately 31.6 mm, with a tolerance of +/−0.2 mm or less (e.g., +/−0.1 mm).

Next, consider that a strap band may be configured to dispose a first subsets of electrodes 4902 at about 61 mm along the strap from center of wearable pod 4950 (e.g., (45 mm/2)+32 mm+4.5 mm (width of 1^{st} electrode)+2.0 mm (half-way between first two electrodes)=61 mm). Also consider, that the second subset of electrodes are located a total of 105.5 mm from the center of wearable pod 4950. In this example, the first subset is disposed about 58% along a curvilinear line (e.g., following the strap) between the center of the wearable pod to the second subset of electrodes. In some embodiments, the first subset of electrodes may be disposed at ratio of 0.45 to 0.70 relative to the distance at which the second subset of electrodes are disposed (e.g., 45% to 70% of the distance).

In view 5320, example dimensions for electrodes 4902 may include a X dimension of 4.5 mm and a Y dimension of 4.5 mm. Electrodes 4902 may have a height Z above inner surface 4900i of strap band 4900 of 1.5 mm. Dimensional tolerances for dimensions X, Y, and Z may be +/−0.2 mm or less (e.g., +/−0.1 mm). In view 5320 dimension W of buckle 4910 may be selected to be greater than dimension Y of electrode 4902 to provide clearance between opposing edges of electrode 4902 and buckle 4910 so that as buckle 4910 slides 4910s along strap band 4900, the buckle 4910 does not make contact with electrodes 4902 (e.g., the opposing edges). Dimension W may be selected to be about 0.3 mm to about 0.6 mm greater than dimension Y of electrodes 4902. For example, if dimension Y is 4.5 mm, then dimension W may be 5.0 mm. Buckle 4910 may include guides 4910g configured to engage with features 4910p on inner surface 4900i of strap band 4900 (see view 5340). For example, prior to attaching loop 4913 to strap band 4900, strap band 4900 may be inserted through an opening 4910o of buckle 4910 and guides 4910g may engage features 4910p to allow indexing (e.g., a mechanical stop) of the buckle 4910 as it slides 4910s along the strap band 4900. The indexing may allow a user of the system 5000 to adjust the fit of the system 5000 to their individual wrist size (e.g., by sliding 4910s the buckle 4910 along strap band 4900), while also providing tactile feedback caused by guides 4910g engaging features 4910p as the buckle slides 4910s along the strap band 4900. Guides 4910g may also be operative to fix the position of the buckle 4910 on the strap band 4900 after the user adjustment has been made so that the buckle 4910 does not move (e.g., buckle 4900 remains stationary unless moved by the user).

Dimensions X, Y, and Z of electrodes 4902 may be selected to determine a surface area of the electrodes 4902 (e.g., for surfaces of electrodes 4902 that are urged into contact with skin in target region 4991). For example, surface area for electrodes 4902 may be in a range from about 10 mm2 to about 20 mm2. In some examples, structure connected with the electrodes 4902 may cover some portion of the surface of the electrodes 4902 and/or sidewall surfaces of the electrodes 4902 and reduce their actual surface area (e.g., skirts 4904 that surround the electrodes 4902, material of strap band 4900). For example, with dimensions X and Y being 4.5 mm such that electrodes 4902 have an actual surface area of 20.25 mm2, an effective surface area of the electrodes 4902 that may be exposed above inner surface 4900i for contact with skin may be 18 mm2.

In view 5340, structure on inner surface 4900i of strap band 4900 is depicted in greater detail than in view 5300. For example, proximate 4915 a portion of dimension B may be arcuate and dimension B may include dimensions B1 and B2, where dimension B1 may be the curved portion of B. The Y dimension for only one of the electrodes 4902 is depicted; however, for purposes of explanation it may be assumed that the Y dimensions of the other electrodes 4902 are identical. In view 5340, strap band 4900 may have a width S of 10.0 mm and a thickness T of 2.0 mm measured between inner 4900i and outer 4900o surfaces. Thickness T may be the thinnest section of strap band 4900 and strap band 4900 may be thicker along portions of dimension B1. Thickness T may be in a range from about 0.9 mm to about 3.2 mm, for example. The following are another example of dimensions in millimeters (mm) for strap band 4900 with example dimensional tolerances of +/−0.2 mm or less (e.g., +/−0.1 mm): dimension B1 may be 16.91 mm; dimension B2 may be 15.02 mm; dimension X for electrodes 4902 may be 4.46 mm; dimension Y for electrodes 4902 may be 4.46 mm; dimension E between adjacent electrodes 4902 may be 3.54 mm; may be 3.54 mm; dimension D (edge-to-edge) may be 32.54 mm or D′ (center-to-center) may be 37.0 mm; and distance C may be 5.96 mm.

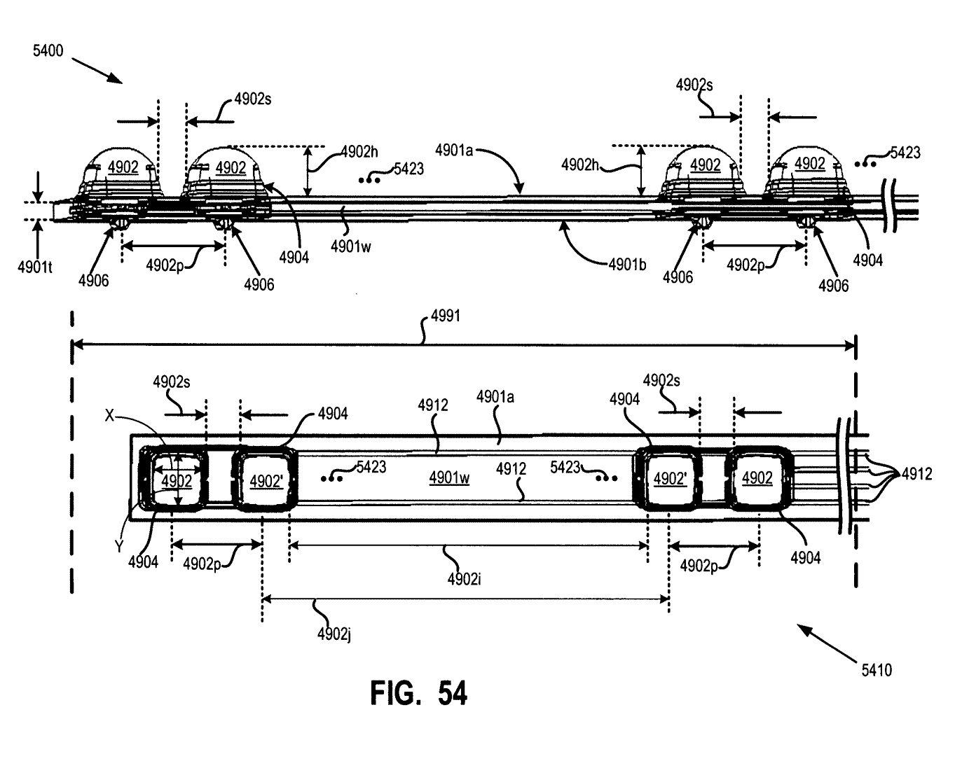

Attention is now directed to FIG. 54 where side view 5400 and top plan view 5410 of a wire bus 4901w is depicted. Wire bus 4901w may be a sub-assembly that is encapsulated (e.g., by injection molding) or otherwise incorporated into strap band 4900. Electrodes 4902 may be mounted on wire bus 4901w and wires 4912 may be connected with electrodes 4902 by a process such as soldering, welding, crimping, for example. Some of the dimensions as described above in regards to FIGS. 51 to 53 may be determined in part by dimensions and placement of electrodes 4902 on wire bus 4901w. As one example a length of wire bus 4901w may be selected to span dimension A of strap band 4900 so that electrodes 4902 on wire bus 4901w are positioned within the target range 4991. Similarly, dimensions B, E, X, Y, D, D′, C, S, and T on strap band 4900 may be determined in part by dimensions, positions and sizes of electrodes 4902 on wire bus 4901w. Wire bus 4901w may be made from a material such as a thermoplastic elastomer (e.g., TPE or TPU). The material for wire bus 4901w may be a flexible material. Wire bus 4901w may have a thickness 4901t in a range from about 0.3 mm to about 1.1 mm, for example. Skirt 4904 may be made from a polycarbonate material, for example.

Electrodes 4902 may include pins 4906 used in mounting the electrodes 4902 to wire bus 4901w. A distance (e.g., a pitch) between centers of pins 4906 may determine the spacing between electrodes 4902 on strap band 4900. For example, spacing 4906 may determine an edge-to-edge distance 4902s between adjacent electrodes 4902 and the distance 4902s may determine distance E on strap band 4900. As another example, an edge-to-edge distance 4902i or a center-to-center distance 4902j between the innermost electrodes 4902′ may determine distances D and D′ respectively on strap band 4900. A height 4902h from a surface 4901a of wire bus 4901w to a top of electrodes 4902 may determine height Z (see view 5320 of FIG. 53) on strap band 4900, for example. Due to the material used to form the strap band 4900 over the wire bus 4901w the dimension for Z will typically be less than the dimension for 4902h. For example, if Z is 1.5 mm, then 4902h may be 1.7 mm. There may be more or fewer electrodes 4902 on wire bus 4901w as denoted by 5423. Skirts 4904 may be coupled with electrodes 4902 and may be operative as an interface between materials for the strap band 4900 and electrodes 4902 and may form a seal around the electrodes 4902. Skirts 4904 and material used to form the strap band 4900 around the wire bus 4901w may reduce actual surface area of the electrodes to an effective surface area as described above.

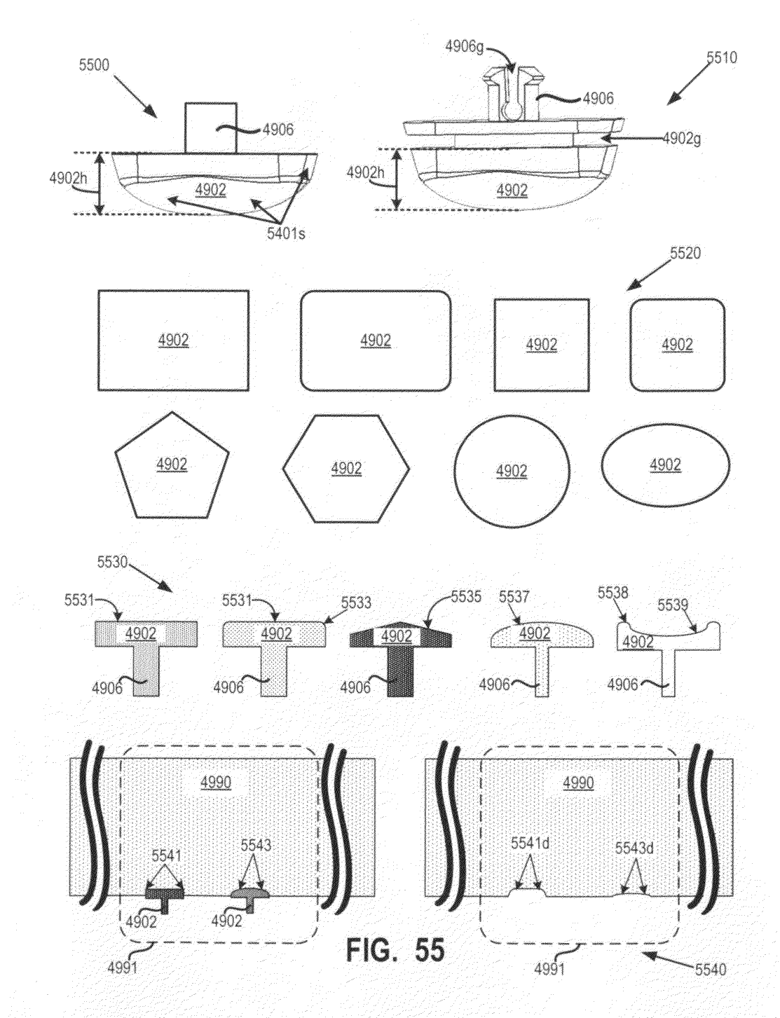

FIG. 55 illustrates various examples of electrodes 4902. In example 5500, electrode 4902 may include an arcuate surface and a pin 4906. Height 4902h may be measured from a top surface to a bottom surface of electrode 4902. In example 5510, electrode 4902 may include a groove 4902g and a pin 4906 that includes a slot 4906g. Height 4902h may be measured from a top surface to a surface of groove 4902g. Groove 4902g may be surrounded by skirt 4904 described above in reference to FIG. 54.

In example 5520, different shaped for electrode 4902 are depicted. Electrode 4902 may have a shape including but not limited to a rectangular shape, a rectangle with rounded corners, a square shape, a square with rounded corners, a pentagon shape, a hexagon shape, a circular shape, and an oval shape, for example.

In example 5530, surfaces of electrode 4902 may have surface profiles including but not limited to a planar surface 5531, a planar surface 5531 with rounded edges 5533, a sloped surface 5535, an arcuate surface 5537 (e.g., convex), and an arcuate surface 5539 (e.g., concave). Arcuate surface 5539 may include rounded edges 5538. Surface profiles of electrodes 4902 may be configured to maximize surface area of the electrodes 4902 that contact skin, to provide a comfortable interface between the electrode and the user's skin (e.g., for prolong periods of use, such as 24/7 use), to maximize electrical conductivity for improved signal to noise ratio (S/N), for example.

In example 5540, electrode 4902 with a planar surface profile 5541 and electrode 4902 having an arcuate surface profile 5543 are depicted engaged with skin of body portion 4990 (e.g., a wrist). After the electrodes 4902 are disengaged with the skin, each electrode 4902 may leave an impression in the skin denoted as 5541d and 5543d. After a period of time has elapsed after the disengaging, the impression 5543d from the electrode 4902 having the arcuate surface profile 5543 may be less pronounced and may fade away faster than the more pronounce impression 5541d left by the electrode 4902 with the planar surface profile 5541. Accordingly, some surface profiles for electrodes 4902 may be more desirable for esthetic purposes (e.g., minimal impression after removal) and for comfort purposes (e.g., sharp edges may be uncomfortable).

Suitable materials for electrodes 4902 include but are not limited to metal, metal alloys, stainless steel, titanium, silver, gold, platinum, and electrically conductive composite materials, for example. Electrodes 4902 may be coated 5401s with a material operative to improve signal capture, such as silver or silver chloride, for example. Electrodes 4902 may be coated 5401s with a material operative to prevent corrosion or other chemical reactions that may reduce electrical conductivity of the electrodes 4902 are damage the material of the electrodes 4902. Examples of substances that may cause corrosion or other chemical reactions include but are not limited to body fluids such as sweat or tears, salt water, chlorine (e.g., from swimming pools), water, household cleaning fluids, etc.

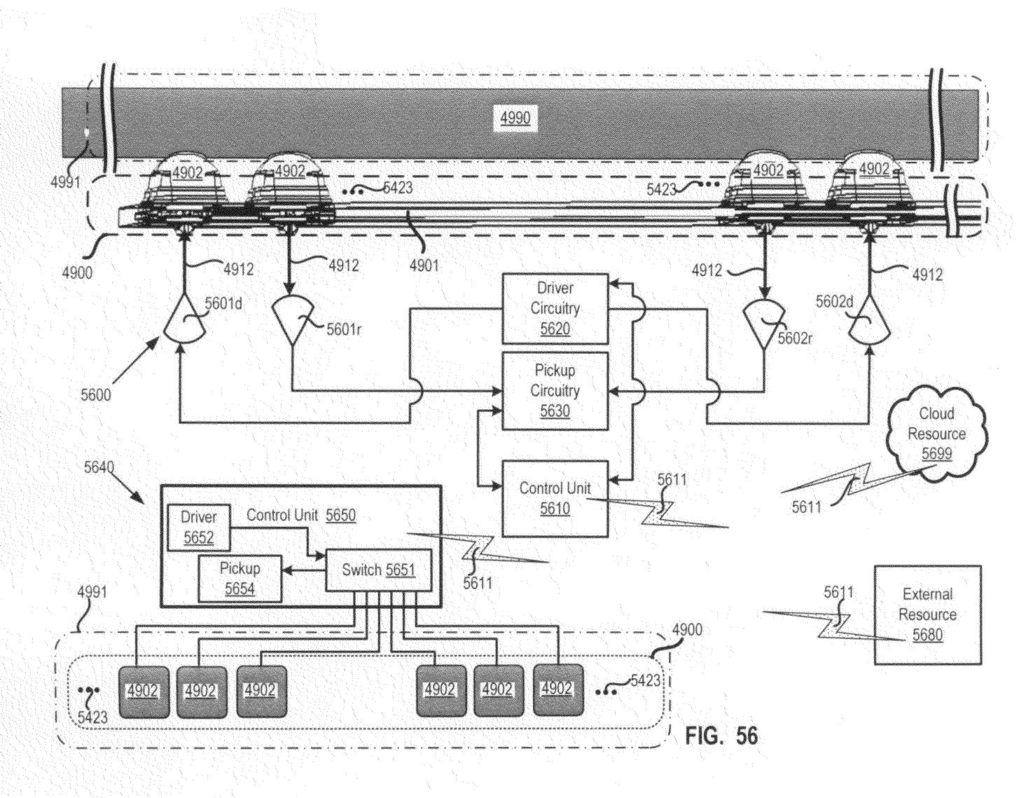

Reference is now made to FIG. 56 where examples of circuitry coupled with electrodes 4902 of a strap band 4900 are depicted. In example 5600, electrodes 4902 are depicted engaged into contact with skin of body portion 4990 within target region 4991. Outermost electrodes 4902 may be coupled (e.g., via wires 4912) with drivers 5601d and 5602d operative to apply a signal to the outermost electrodes 4902 (e.g., driven D electrodes 4902). Innermost electrodes 4902 may be coupled (e.g., via wires 4912) with receivers 5601r and 5602r operative to receive signals picked up by innermost electrodes 4902 from electrical activity on the surface of and/or within body portion 4990. Drivers 5601d and 5602d may be coupled with driver circuitry 5620 and receivers 5601r and 5602r may be coupled with pickup circuitry 5630. A control unit 5610 may be coupled with driver circuitry 5620 and with pickup circuitry 5630. Control unit 5610 may include one or more processors, data storage, memory, and algorithms operative to control driver circuitry 5620 and pickup circuitry 5630 to process data received by pickup circuitry 5630, and to generate data used by driver circuitry 5620 to output driver signals coupled with drivers 5601d and 5602d, for example. As one example, electrodes 4902 may sense and/or generate signals associated with biometric functions of the body, such as bioimpedance (BI). Control unit 5610 may perform signal processing of signals associated with driver circuitry 5620 and/or pickup circuitry 5630, or an external resource 5680 and/or cloud resource 5699 in communication 5611 (e.g., via a wired or wireless communication link) may perform some or all of the processing. For example, control unit 5610 may transmit 5611 data to 5680 and/or 5699 for processing. External resource 5680 and/or cloud resource 5699 may include or have access to compute engines, data storage, and algorithms that are used to perform the processing.

In example 5640, strap band 4900 may include a plurality of electrodes 4902 coupled with a switch 5651 that is controlled by a control unit 5650. Control unit 5650 may command switch 5651 to couple one or more of the electrodes 4902 with driver circuitry 5652 such that electrodes 4902 so coupled become driven electrodes D. Control unit 5650 may command switch 5651 to couple one or more of other electrodes 4902 with pickup circuitry 5654 such that electrodes 4902 so coupled become pick-up electrodes P. There may be more or fewer of the electrodes 4902 as denoted by 5423. Processing of signals and/or data may be handled by control unit 5650 and/or by external resource 5680 and/or cloud resource 5699 using communications link 5611 as described above. Algorithms and/or data used in the processing may be embodied in a non-transitory computer readable medium (e.g., non-volatile memory, disk drive, solid state drive, DRAM, ROM, SRAM, Flash memory, etc.) configured to execute on one or more processors, compute engines or other compute resources in control unit 5610, 5650, external resource 5680 and cloud resource 5699. Electrodes 4902 in example 5640 may be used to cover additional surface area on body portion 4990 as may be needed to accommodate differences in size of body portion 4990 among a user population. External resource 5680 may be a wireless client device, such as a smartphone, tablet, pad, PC or laptop and may execute an algorithm or application (APP) operative to determine which electrodes 4902 to activate via switch 5651 as driver D or pick-up P electrodes. A user may enter information about their wrist size or other body portion size as data used by the APP to make electrode 4902 selections. Control unit 5610 and/or 5650 may be included in device 4950 of FIG. 50, for example.

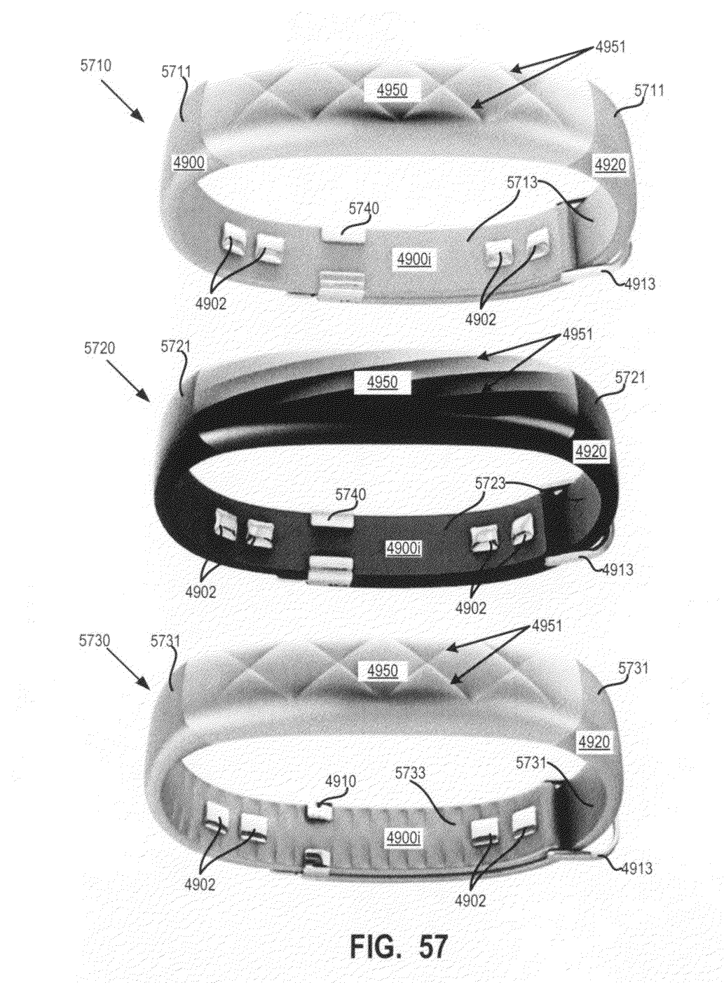

FIG. 57 illustrates profile views of systems 5710-5730 that include strap band 4900. System 5710 may include device 4950, band 4920, and strap band 4900. Band 4920 and strap band 4900 may be made from a thermoplastic elastomer such as TPE, TPU, TPSV, or others, for example. The thermoplastic elastomer may be covered with an exterior fabric material 5711, such as cloth or nylon, for example. The electrode 4902 and fastening hardware 4913, 4921, 5740 may be anodized or coated with a surface finish such as a colored chrome finish, for example. In system 5710, buckle 4910 may be replaced with a buckle 5740 configured to slide 4910s along the exterior fabric material 5711 without damaging the fabric material 5711.

System 5720 may include a faux leather exterior surface material 5721 which may have a variety of finishes such as matte, flat, glossy, etc. The finishing layer can be added prior to molding. An example of synthetic leather is known as “leatherette,” among others. The fastening hardware of system 5720 may be coated with a surface finish as described above.

System 5730 includes band 4920 and strap band 4900 that may be from a material 5731, such as a thermoplastic elastomer such as TPE, TPU, TPSV, or others, for example. Inner surface 4900i of strap band 4900 includes features operative to index buckle 4910 as was described above in reference to FIG. 53. Material 5721 which may have a variety of finishes such as matte, flat, glossy, etc. The fastening hardware of system 5730 may be coated with a surface finish as described above. Device 4950 may include top and bottom portions made from a material such as anodize aluminum that may be anodized in a variety of colors, for example. An upper surface may include ornamental elements 4951.

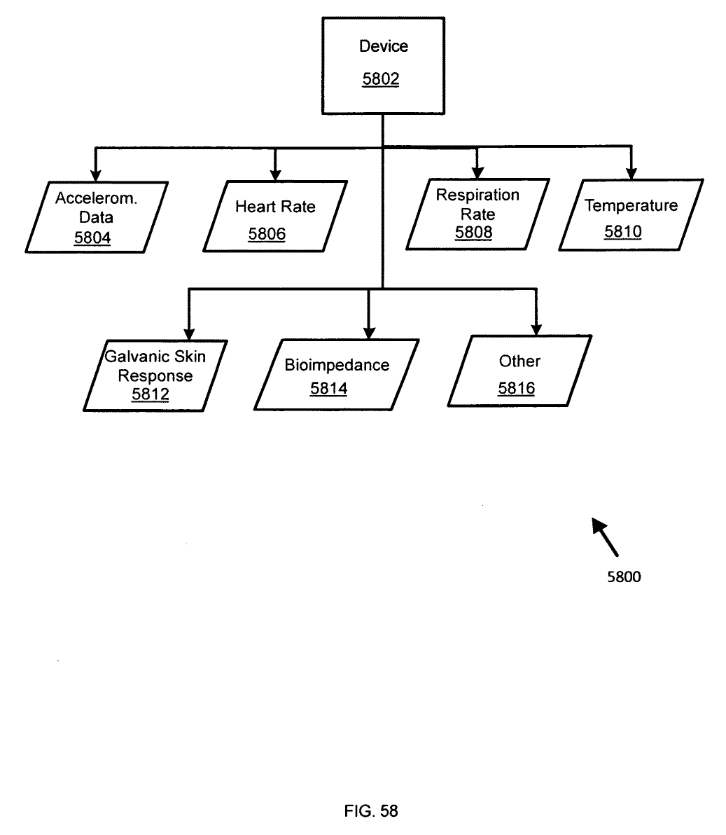

FIG. 58 illustrates exemplary data types for device-based activity classification using predictive feature analysis. In some examples, data group 5800 depicts various types of data that may be received, processed, modified, operated upon, manipulated, stored, operated, transmitted, transceived, or otherwise used by device 5802. While some data types are shown, there are other data types that may be used beyond those shown and described. Here, device 5802 may be implemented with one or more sensors of various types, including, but not limited to accelerometer, temperature, galvanic skin response, bioimpedance, digital, analog, or any other type, configuration, or quantity of sensor beyond those described. While not shown, sensors implemented with device 5802 may be configured to detect signals from a user or wearer (or from tissue or a body on which device 5802 may be, in some examples, worn) or a surrounding environment to which sensors (not shown) onboard device 5802 may be exposed. As used herein, a sensor may be implemented as a single or multiple sensors that can be implemented using any type of sensor, sensory device, circuit, or other implementation that is configured to detect an input and, once detected, transmit or transceiver a corresponding signal to device 5802, a processor (e.g., local or remote; not shown), or any other destination configured to receive signals that be interpreted and/or used to derive data, such as those shown in FIG. 58.

Here, some data types may be derived or determined from signals that indicate motion (e.g., accelerometer data 5804), heart rate (5806), respiration rate (5808), temperature (5810), galvanic skin response (5812), bioimpedance (5814), or other types of data (5816), including, but not limited to salinity, barometric pressure, vapor detection, carbon monoxide, carbon dioxide, outgassing, or others, without limitation. Various types of data may be derived from signals received from sensors (e.g., sensors coupled or implemented with device 5802) after performing one or more digital or analog operations on signals obtained from a sensor or sensor array (i.e., multiple sensors implemented locally, remotely, or distributed using device 5802 and other devices (not shown)).

In some examples, the described techniques, here and below, may be implemented as a computer program or application (“application”) or as a plug-in, module, or sub-component of another application. The described techniques may be implemented as software, hardware, firmware, circuitry, or a combination thereof. If implemented as software, the described techniques may be implemented using any type of structured or unstructured programming, development, compiling, scripting, or formatting languages or programs, frameworks, syntax, applications, protocols, objects, or techniques, including, but not limited to, FORTH, ASP, ASP.net, .Net framework, Ruby, Ruby on Rails, C, Objective C, C++, C#, Adobe® Integrated Runtime™ (Adobe® AIR™), ActionScript™, Flex™, Lingo™, Java™, Javascript™, Ajax, Perl, COBOL, Fortran, ADA, XML, MXML, HTML, DHTML, XHTML, HTTP, XMPP, PHP, and others. Design, publishing, and other types of applications such as Dreamweaver®, Shockwave®, Flash®, Drupal and Fireworks® may also be used to implement the described techniques. Database management systems (i.e., “DBMS”), search facilities and platforms, web crawlers (i.e., computer programs that automatically or semi-automatically visit, index, archive or copy content from, various websites (hereafter referred to as “crawlers”)), and other features may be implemented using various types of proprietary or open source technologies, including MySQL, Oracle (from Oracle of Redwood Shores, Calif.), Solr and Nutch from The Apache Software Foundation of Forest Hill, Md., among others and without limitation. The described techniques may be varied and are not limited to the examples or descriptions provided.

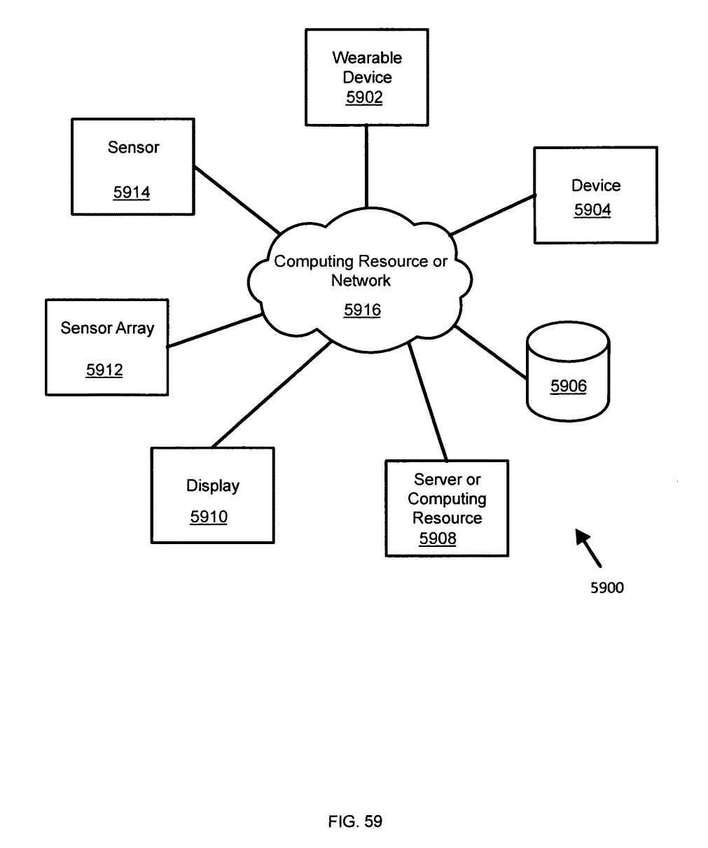

FIG. 59 illustrates an exemplary computing network topology for device-based activity classification using predictive feature analysis. In some examples, topology 5900 may include wearable device 5902, device 5904, repository (e.g., data repository, storage, memory, facility, or the like) 5906, server/computing resource 5908, display 5910, sensor array 5912, sensor 5914, and computing resource/network 5916. In other examples, the number, type, configuration, and implementation of elements 5902-5916 may be varied and are not limited to the examples shown.

Here, wearable device 5902 may be implemented as a data-capable band, such as those shown and described herein (e.g., FIG. 57). Other types of devices (e.g., device 5904) may also be used with the techniques described herein and are not limited. As shown and described wearable device 5902 and device 5904 may be implemented having one or more sensors (not shown) or sensor arrays that may, for example, include electrode(s) that are configured, formed, adapter, or otherwise implemented to detect a signal using, among other techniques, bioimpedance (i.e., detecting resistance (in both magnitude and phase) to an electrical current introduced into a biologic body or tissue (e.g., a body, arm, leg, appendage, or the like) that can be used, alone or in connection with the techniques described herein, to provide, generate, or otherwise produce data associated with various types of biological parameters such as heart rate, respiration rate, galvanic skin response, temperature, and many others, without limitation. Here, wearable device 5902 and device 5904 may be in data communication with each other or other devices over computing resource/network (e.g., data network, computing cloud, local area network (LAN), wide area network (WAN), or the like, without limitation) 5916. As described, any type of sensor, including sensor 5914, or sensor array 5912 (e.g., multiple sensors implemented together to provide a single sensory receptacle or receiver or as a “battery” of sensors, each of which may be adapted to detect the same or dissimilar types of signals) may be implemented and are not limited to those shown and described. In some examples, signals may be received as input by sensors implemented with wearable device 5902 or device 5904, or by sensor array 5912 and sensor array 5914 and, upon sensing or detection, may be converted (e.g., analog-to-digital, digital-to-analog, digital-to-digital, and the like), translated, rectified or otherwise modified to generate data that may be stored in repository 5906 or used by server/computing resource 5908 and sending or receiving resulting data over computing source/network 5916. In other examples, server/computing resource 5908 may be a node, server, computer, application, or other local, networked, or distributed computing facility or resource that may be used to evaluate a signal sensed by, for example, wearable device 5902, device 5904, sensor 5914, or sensor array 5914. Evaluating the sensed signal may result in data that is generated and used to indicate various types of conditions or parameters associated with a user or wearer (e.g., heart rate, respiration rate, temperature, galvanic skin response, salinity, outgassing, and others). In other examples, different topologies having more, fewer, or different types of elements apart from those shown and described may be implemented, without limitation.

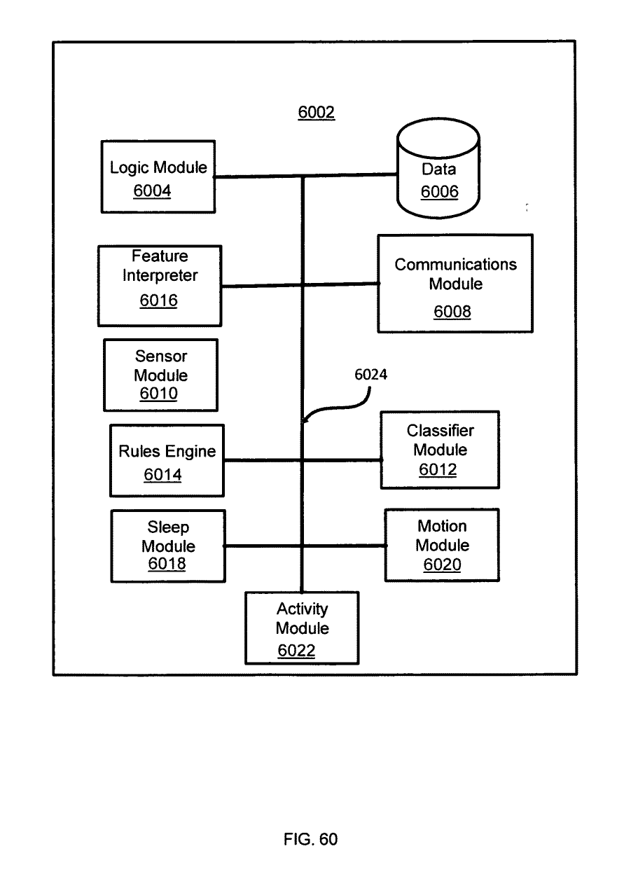

FIG. 60 illustrates an exemplary application architecture for device-based activity classification using predictive feature analysis. Here, application 6002 may be implemented as a standalone or distributed computer program, software, circuit, or other type of computing resource. As shown, application 6002 includes logic module 6004, data repository 6006, communications module 6008, sensor module 6010, classifier module 6012, rules engine 6014, feature interpreter 6016, sleep module 6018, motion module 6020, or activity module 6022. In some examples logic module 6004 may be implemented as a single or multiple processors, including as software, firmware, hardware, or circuitry, without limitation and configured to process data received by application 6002 using, for example, communications module 6008. Communications module 6008, in some examples, may be configured to provide data communication capabilities using any type of digital or analog communication technique, protocol, or facility such as Bluetooth®, BTLE (Bluetooth® Low Energy), near field communication (NFC), RFID (radio frequency identification), WiFi (using various types of data communication protocols such as any variant of the IEEE 802.11 standard (e.g., a, b, c, g, n, and others, without limitation)), or others. As shown, sensor module 6010 may be configured to provide signal and signal data directly to logic module 6004 and application 6002 or, indirectly, using communications module 6008 using bus 6024. Although shown, bus 6024 is not required to transfer data between any of the elements shown and is provided for purposes of illustrating an exemplary technique for transferring data between elements. In other examples, application architecture 6000 is used to illustrate representative processing functions within application 6002 using detected signals (e.g., signals detected by sensors or sensor arrays (not shown) in electrical or data communication with sensor module 6010) or data received from sensors or sensor arrays (not shown). Here, sensor module 6010 may be configured to manage one or more sensors that are used by application 6002 to provide input to logic module 6004, for example, to classify various types of detected activities based on signals received for individual components of activities such as counted steps, three-dimensional motion, temperature, galvanic skin response, temperature (body or environmental), heart rate, respiration rate, bioimpedance-based signals that can be interpreted using feature interpreter 6010 to perform various operations such as algorithmic comparisons or decisions to determine whether quantitative thresholds have been met or exceeded in order to identify and associate specific activities with detected signals. Examples of types of activities that may be detected, evaluated, and classified by “classifiers” such as sleep module 6018, motion module 6020, and activity module 6022. As used herein, classifier may be used to refer to any type of application, computer program, module, engine, circuit, or logic that may be designed or implemented to classify an activity by evaluating a signal detected by a sensor or sensor array such as those described herein and in connection with bands such as those described above. Further, activity classification may be refined using a single or multiple signals, such as the detection of signals from an accelerometer, a bioimpedance circuit, temperature sensor, salinity sensor, galvanic skin response (GSR) sensor, and many others, without limitation. For example, an accelerometer may be configured to detect a signal that could identify motion is occurring using, for example, wearable device 5902 (FIG. 59). Once detected, classifier module 6012 may activate other sensors (e.g., a bioimpedance sensor) to detect heart rate or respiration rate, among other bodily parameters, to further refine an evaluation of signals received by sensors on wearable device 5902, to more precisely or specifically identify the type of motion (e.g., running, walking, skipping, galloping, sprinting, and the like) that is occurring. Where single sensor devices such as accelerometer-only fitness “trackers” can detect motion, the described techniques may be configured to provide a highly resolved degree of accuracy as to the type, category, degree, or other aspects of a given activity. In other words, by using feature interpreter 6016 to determine what rules to apply using, for example, rules stored in rules engine 6014 or data repository 6006, rapid processing of detected signals may be used from multiple sensors to provide accurate detection, identification, and classification of activities ranging from running and walking to sleep, among many others.

As described herein, feature interpreter 6016 may be implemented as an application, software, or firmware that is configured to interpret various types of “predictive features” that can be used to identify and classify particular types of activities. For example, if sensor module 6010 receives a signal that indicates heart rate, feature interpreter 6016 may execute or apply a rule from rules engine 6014 that suggests that if the detected heart rate exceeds a given threshold, then a classifier for running, walking, or locomotion (e.g., motion module 6020) should be “activated” (i.e., invoked, executed, instanced, called, run, instantitated, triggered, “turned on,” or the like) in order to classify the detected motion. In other examples, if a detected signal indicates that a user's heart rate has fallen below a given threshold, feature interpreter 6016 may be configured to activate sleep module 6018 in order to determine whether a “state” (e.g., physical, physiological, psychological, anatomical, emotional, chemical, biochemical, mechanical, biomechanical, or others) of a wearer of, for example, wearable device 5902, is light sleep, deep sleep, or sleep associated with rapid eye movement (REM). In other examples, state determination may also be performed using other types of classifiers (e.g., motion module 6020 or activity module 6022, the latter of which may be used to implement classification logic for any type of activity using any type of sensor input beyond those shown and described herein.

In some examples, feature interpreter 6016 may be implemented as an application that is configured to determine a state associated with a given user by applying one or more rules managed by rules engine 6014. Examples or rules may include, but are not limited to, logic that is embedded or implemented with other software or firmware stored on and executed by, for example, wearable device 5902 or device 5904. Some rules may include determining when a sleep state is determined for a given number of time periods (i.e., a set quantity or amount of time may be used to establish a threshold below which, if no motion is detected or if a lowered heart rate or respiration rate are detected, sleep is occurring, thus triggering feature interpreter 6016 to further determine and classify the type of sleep (e.g., light, deep, REM, waking, rousing, or others)). Other rules may include predicting when sleep module 6018 (i.e., a classifier) predict a user's state has changed from sleeping to waking, then motion module 6020 (i.e., which may also be referred to as a “step classifier”) may be activated in order to begin tracking steps or motion using, for example, an accelerometer-based sensor, in addition to a bioimpedance-based sensor that was used to initially detect an increasing heart rate or respiration rate to indicate waking. Other rules may be implemented, without limitation, including those that may not only be used to classify activities, but to also manage other aspects or conditions associated with wearable device 5902 or device 5904, such as power consumption or conservation by activating or deactivating (i.e., turning on or off) a given sensor, set of sensors, sensor array, electrodes, or the like. Further, rules managed by rules engine 6014 may also be used to manage, for example, the operation of various types of sensors based on predictive features. For example, if sleep is detected by a sensor adapted to detected bioimpedance-related signals, wearable device 5902 may be configured or modified by logic module 6002 to lessen, lower, or altogether stop the generation and transmission of electrical currents (which may be of any current or voltage used to implement bioimpedance related electrode measurement such as driving small (e.g., fractions of a micro amp of electrical current) amounts of electrical current into tissue, bone, or other biological structures to sense impedance for purposes of determining heart rate or respiration rate. In addition to rules that may be used to manage sensor operation/disabling/suspension and activity classification, other rules managed by rules engine 6014 may be implemented without limitation and are not confined to those described.

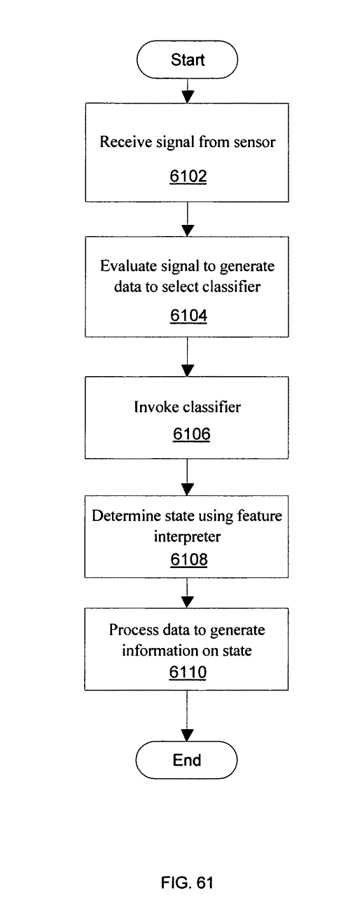

FIG. 61 illustrates an exemplary process for device-based activity classification using predictive feature analysis. Here, a signal is received (e.g., detected) by a sensor, which may be implemented using any of the sensor types or techniques described herein (6102). The detected signal may be evaluated, in some examples, to generate data that can be further evaluated in order to determine whether a classifier (i.e., firmware, software, computer program, application used to evaluate data to classify a given activity based on the detected signal(s)) should be activated for a given detection (6104). This may also include identifying the type of activity in order to select a classifier that can, using input (e.g., other detected signals) from other sensors, to classify the activity (6106). Upon evaluation, a classifier may be invoked and, using data generated from the initially detected signal, receive other signals or detected associated with other detected signals to determine a state associated with wearable device 5902 or device 5904 and a given user (6108). For example, an accelerometer may determine that a user has not moved for an extended period of time (in some examples, a period of time may be referred to as a “press”) and, using feature interpreter 6016 (FIG. 60), a bioimpedance sensor or circuit may be activated in order to detect other signals that can invoke a sleep classifier (e.g., sleep module 6018 (FIG. 60)) to further determine whether the detected state (i.e., sleep) is light, deep, or REM-based sleep. In other examples, different classifiers may be invoked in order to identify or define with greater accuracy a given activity. Once determine, data associated with a given state (i.e., state data) may be further processed to create or generate an output representation (e.g., graphical, haptic, luminescent, vibratory, or others, without limitation) to an interface, which may be implemented using any of the techniques described herein. In still other examples, the above-described process may be varied in order, steps, function, or other aspects, without limitation to the examples shown and described.

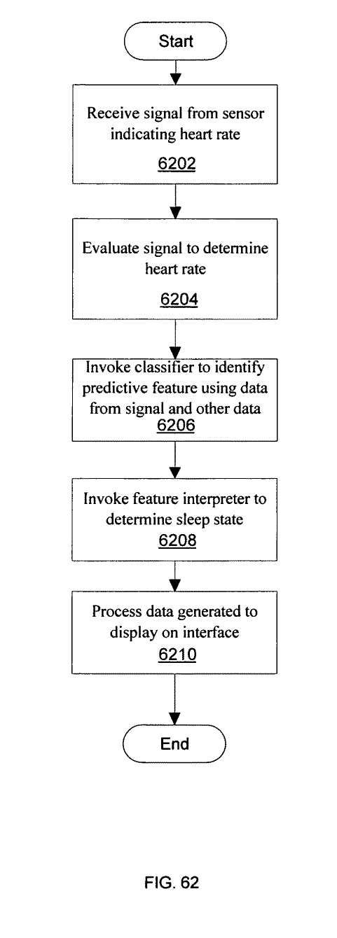

FIG. 62 illustrates another exemplary process for device-based activity classification using predictive feature analysis. Here, an exemplary process for sleep classification is shown, including receiving a signal from a sensor indicating or measuring heart rate (e.g., bioimpedance, optical, acoustic, or others) (6202). The received signal is evaluated to determine a measurement for the detected heart rate (6204). Once determined, the heart rate may be further evaluated by feature interpreter 6016 (FIG. 60) to identify and invoke a classifier for further classifying the detected activity (e.g., light sleep, deep sleep, REM sleep, and the like) (6206-6208). Finally, the data may be processed to generate information to be provided via an interface such as those described above. This information may be reviewed by a user (e.g., of wearable device 5902 (FIG. 59)) to evaluate the nature, quality, duration, type, or other characteristics of his/her sleep. In still other examples, the above-described process may be varied in order, steps, function, or other aspects, without limitation to the examples shown and described.

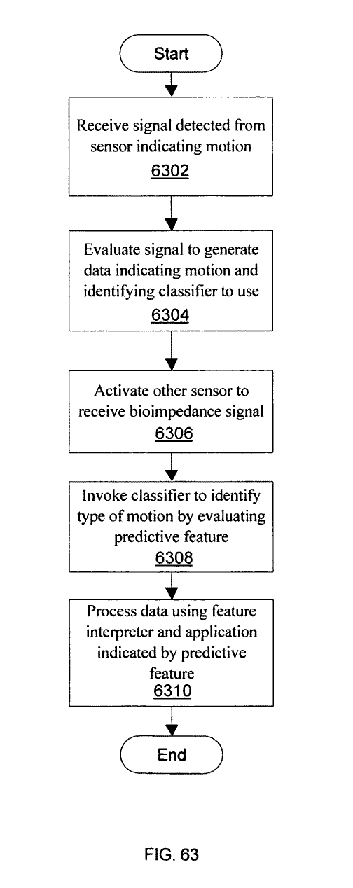

FIG. 63 illustrates a further exemplary process for device-based activity classification using predictive feature analysis. Here, a signal may be received from a sensor indicating motion has occurred (6302). The received signal may be evaluated to generate data indicating one or more characteristics or parameters associated with the motion that may be used by feature interpreter 6016 (FIG. 60) to activate another sensor to receive, for example, bioimpedance signals (6304-6306). Although the type of sensor indicated here is bioimpedance-related, other types of sensors may be used and are not limited to those described herein, which are provided solely for purposes of illustrating the described techniques. Once activated, other signals are detected and evaluated to generate additional data that is used to identify and select a classifier (e.g., sleep module 6018, motion module 6020, activity module 6022, and others) that, once invoked, may be used to classify (i.e., further identify) the type of motion (6308). Finally, the data generated from the initially detected signal and the bioimpedance-related signal may be processed to generate information to present on an interface that may be implemented locally on, for example, wearable device 5902 or device 5904, or on a remote device (e.g., display 5910 (FIG. 59)) that is in data communication with a given device. Various types of interface technologies may be used and are not limited to those shown and described. In yet other examples, the above-described process may be varied in order, steps, function, or other aspects, without limitation to the examples shown and described.

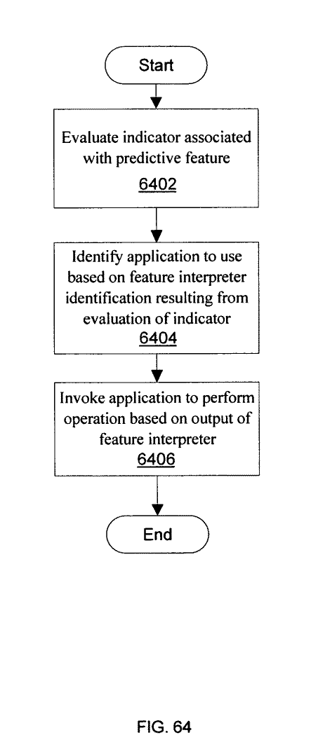

FIG. 64 illustrates yet another exemplary process for device-based activity classification using predictive feature analysis. Here, an exemplary process for feature interpretation as implemented by feature interpreter 6016 (FIG. 60) is shown. In some examples, an indicator associated with a predictive feature (e.g., a signal detected by a sensor) is evaluated (6402). Once evaluated, an application such as a classifier (e.g., sleep module 6018, motion module 6020, activity module 6022, and others) is identified (6404). Once identified the classifier (i.e., application) is invoked by feature interpreter 6016 to perform a further evaluation or data operation (e.g., compare or other data operation configured to algorithmically evaluate data to determine whether a rule or set of rules such as those managed by rules engine 6014 (FIG. 60) should be applied in order to generate an activity classification) (6406). The above-described process may be varied in order, steps, function, or other aspects, without limitation to the examples shown and described.

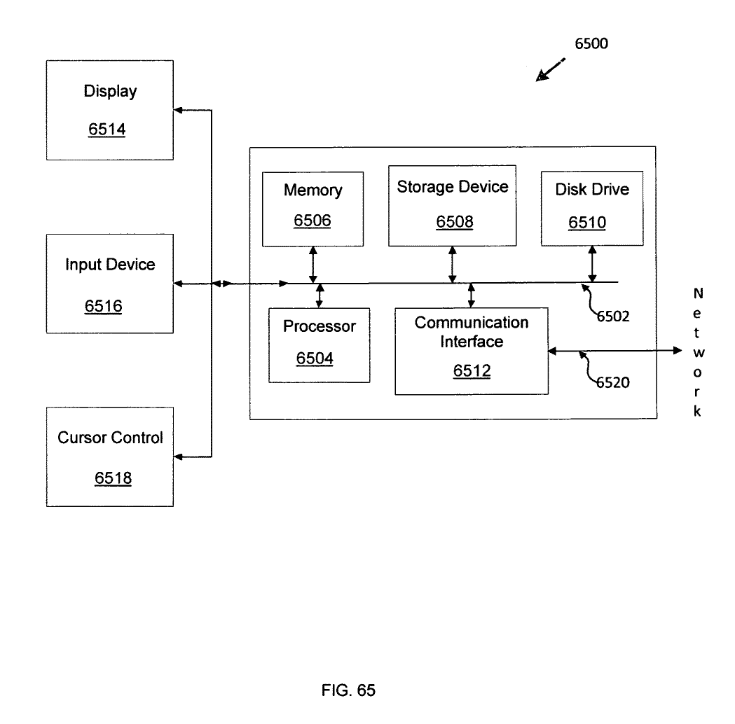

FIG. 65 illustrates an exemplary computer system suitable for device-based activity classification using predictive feature analysis. In some examples, computer system 6500 may be used to implement computer programs, applications, methods, processes, or other software to perform the above-described techniques. Computer system 6500 includes a bus 6502 or other communication mechanism for communicating information, which interconnects subsystems and devices, such as processor 6504, system memory 6506 (e.g., RAM), storage device 6508 (e.g., ROM), disk drive 6510 (e.g., magnetic or optical), communication interface 6512 (e.g., modem or Ethernet card), display 6514 (e.g., CRT, LED, LCD, plasma, OLED, etc.), input device 6516 (e.g., keyboard), and cursor control 6518 (e.g., mouse or trackball).

According to some examples, computer system 6500 performs specific operations by processor 6504 executing one or more sequences of one or more instructions stored in system memory 6506. Such instructions may be read into system memory 6506 from another computer readable medium, such as static storage device 6508 or disk drive 6510. In some examples, hard-wired circuitry may be used in place of or in combination with software instructions for implementation.

The term “computer readable medium” refers to any tangible medium that participates in providing instructions to processor 6504 for execution. Such a medium may take many forms, including but not limited to, non-volatile media and volatile media. Non-volatile media includes, for example, optical or magnetic disks, such as disk drive 6510. Volatile media includes dynamic memory, such as system memory 6506.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 6502 for transmitting a computer data signal.

In some examples, execution of the sequences of instructions may be performed by a single computer system 6500. According to some examples, two or more computer systems 6500 coupled by communication link 6520 (e.g., LAN, PSTN, or wireless network) may perform the sequence of instructions in coordination with one another. Computer system 6500 may transmit and receive messages, data, and instructions, including program, i.e., application code, through communication link 6520 and communication interface 6512. Received program code may be executed by processor 6504 as it is received, and/or stored in disk drive 6510, or other non-volatile storage for later execution

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method, comprising:
receiving a signal from a sensor configured to measure a heart rate coupled to a device, the sensor being configured to sense the signal over a time period;
evaluating the signal to generate data associated with the heart rate, the data being further evaluated to select a classifier;
invoking the classifier, the classifier being configured to evaluate the data to identify a predictive feature, the predictive feature invoking an application configured to determine a state using a feature interpreter, the application also being configured to evaluate other data from another signal, the signal being configured to detect a respiration rate; and
processing the data and the other data using the application and the feature interpreter to generate information associated with sleep, the information being configured to display on an interface associated with the device.

2. The method of claim 1, wherein the signal is associated with bioimpedance.

3. The method of claim 1, wherein the sensor is configured to detect bioimpedance.

4. The method of claim 1, wherein the sensor comprises one or more electrodes, each of the one or more electrodes being configured to detect the heart rate or a respiration rate.

5. The method of claim 1, wherein the classifier comprises an application configured to perform an operation at one or more levels of an application protocol stack, the operation using, as input, the signal and the another signal.

6. The method of claim 1, wherein the classifier comprises an application configured to perform an operation at one or more levels of an application protocol stack, the operation using, as input, the signal and the another signal, wherein the signal indicates a heart rate and the another signal indicates a respiration rate.

7. The method of claim 1, wherein the classifier comprises an application configured to perform an operation at one or more levels of an application protocol stack, the operation using, as input, the signal and the another signal, wherein the signal indicates a heart rate and the another signal indicates a motion.

8. The method of claim 1, wherein the classifier comprises an application configured to perform an operation at one or more levels of an application protocol stack, the operation using, as input, the signal and the another signal, wherein the signal indicates a heart rate and the another signal indicates a galvanic skin response.

9. The method of claim 1, wherein the another signal is detected by another sensor, the another sensor being configured to detect bioimpedance.

10. The method of claim 1, wherein the feature interpreter is configured to perform an operation at one or more layers of an application protocol stack using the data, the operation being used to identify one or more parameters associated with the state.

11. The method of claim 1, wherein the feature interpreter is configured to perform an operation using the signal and another operation using another signal, the operation being performed to identify the sleep and the another operation being performed to identify a type associated with the sleep.

12. The method of claim 1, wherein the classifier is configured to determine whether the sleep is associated with rapid eye movement.

13. The method of claim 1, wherein the classifier is configured to determine whether the sleep is associated with deep sleep.

14. The method of claim 1, wherein the classifier is configured to evaluate the heart rate and the respiration rate to classify the sleep.

15. The method of claim 1, wherein the other sleep is associated with the respiration rate.

16. The method of claim 1, wherein the classifier is configured to evaluate the data to detect sleep, the classifier being further configured to evaluate the other data to determine whether the sleep is associated with rapid eye movement.

17. The method of claim 1, wherein the classifier is configured to evaluate the data to detect sleep, the classifier being further configured to evaluate the other data to determine whether the sleep is deep sleep.

18. A system, comprising:
a memory configured to store data associated with a signal and other data associated with another signal; and
a processor configured to receive the signal from a sensor configured to measure a heart rate coupled to a device, the sensor being configured to sense the signal over a time period, to evaluate the signal to generate data associated with the heart rate, the data being further evaluated to select a classifier, to invoke the classifier, the classifier being configured to evaluate the data to identify a predictive feature, the predictive feature invoking an application configured to determine a state using a feature interpreter, the application also being configured to evaluate the other data from the another signal, the signal being configured to detect a respiration rate, and to process the data and the other data using the application and the feature interpreter to generate information associated with sleep, the information being configured to display on an interface associated with the device.

19. A computer readable medium including instructions for performing a method, the method comprising:
receiving a signal from a sensor configured to measure a heart rate coupled to a device, the sensor being configured to sense the signal over a time period;
evaluating the signal to generate data associated with the heart rate, the data being further evaluated to select a classifier;
invoking the classifier, the classifier being configured to evaluate the data to identify a predictive feature, the predictive feature invoking an application configured to determine a state using a feature interpreter, the application also being configured to evaluate other data from another signal, the signal being configured to detect a respiration rate; and
processing the data and the other data using the application and the feature interpreter to generate information associated with sleep, the information being configured to display on an interface associated with the device.
